(19) United States
(12) Patent Application Publication
(10) Pub. No.: US 2025/0340573 A1
(43) Pub. Date: Nov. 6, 2025

(54) THE SYNTHESIS OF OMAPATRILAT

(71) Applicant: Endothelium Scanning Nanotechnology Limited, Melbourne (AU)

(72) Inventors: Romina VITALE, Groningen (NL); Vincent Alexandre Berthole MARTIN, Groningen (NL); Wijnand S. FABER, Groningen (NL); Roman SOMMER, Groningen (NL)

(21) Appl. No.: 19/208,346

(22) Filed: May 14, 2025

Related U.S. Application Data
(63) Continuation of application No. PCT/AU2023/051168, filed on Nov. 17, 2023.
(60) Provisional application No. 63/426,179, filed on Nov. 17, 2022.
(60) Provisional application No. 63/545,898, filed on Oct. 26, 2023.

(51) Int. Cl. C07D 513/04; A61K 31/554; C07D 317/30

(52) U.S. Cl. CPC C07D 513/04; A61K 31/554; C07D 317/30

(57) ABSTRACT

Described herein are improved methods of making Compound 1 (4S,7S,10aS)-4-((S)-2-mercapto-3-phenylpropanamido)-5-oxooctahydro-7H-pyrido[2,1-b][1,3]thiazepine-7-carboxylic acid, or Omapatrilat, and purified Omapatrilat obtained from the improved methods.

### CROSS REFERENCE TO RELATED APPLICATIONS

This application is a continuation of International Application No. PCT/AU2023/051168, filed Nov. 17, 2023, which claims the benefit of U.S. Provisional Application No. 63/426,179, filed on Nov. 17, 2022, and U.S. Provisional Application No. 63/545,898, filed on Oct. 26, 2023, each of which is incorporated herein by reference in its entirety.

### BACKGROUND

Compound 1 ((4S,7S,10aS)-4-((S)-2-mercapto-3-phenylpropanamido)-5-oxooctahydro-7H-pyrido[2,1-b][1,3]thiazepine-7-carboxylic acid, or Omapatrilat) is a vasopeptidase inhibitor that acts on angiotensin converting enzyme (ACE) and neprilysin (NEP). Through its dual action, Compound 1 can induce vasodilation, which useful in treating hypertension. Described herein is an improved method to produce Compound 1 with increased yields and optical purity.

### SUMMARY

Disclosed herein in some embodiments, is Compound 1, a drug candidate in development for the treatment of diseases or conditions that would benefit from treatment with a dual neprilysin (neutral endopeptidase or NEP) and angiotensin converting enzyme (ACE) inhibitor, such as hypertension. In some embodiments, disclosed herein is purified Compound 1. Described herein is an improved method for the reliable production of Compound 1 (Omapatrilat) with increased yields and optical purity.

In one aspect, disclosed herein is a purified compound having the structure of (4S,7S,10aS)-4-((S)-2-mercapto-3-phenylpropanamido)-5-oxooctahydro-7H-pyrido[2,1-b][1,3]thiazepine-7-carboxylic acid (Compound 1) or a pharmaceutically acceptable salt thereof:

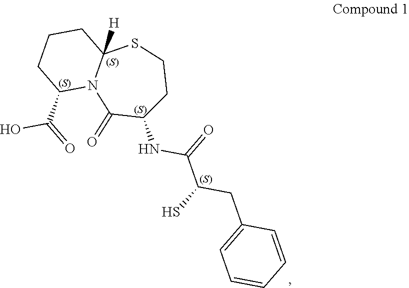

wherein
(i) the compound purity is greater than 97.0% as determined by chromatographic analysis at 215 nm;
(ii) the total amount of any one impurity is less than 1.5% as determined by chromatographic analysis at 215 nm;
(iii) the total content of all impurities is less than 3.0% as determined by chromatographic analysis at 215 nm; or
(iv) combinations thereof.

In some embodiments, the compound purity is greater than 97% as determined by chromatographic analysis at 215 nm. In some embodiments, the total amount of any one impurity is less than 1.5% as determined by chromatographic analysis at 215 nm. In some embodiments, the total content of all impurities is less than 3.0% as determined by chromatographic analysis at 215 nm. In some embodiments, the compound is substantially free of

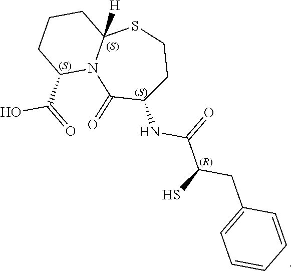

In some embodiments, the compound has optical purity of greater than about 98% enantiomeric excess. In some embodiments, the impurity or impurities comprises one or more of the impurities selected from the group consisting of

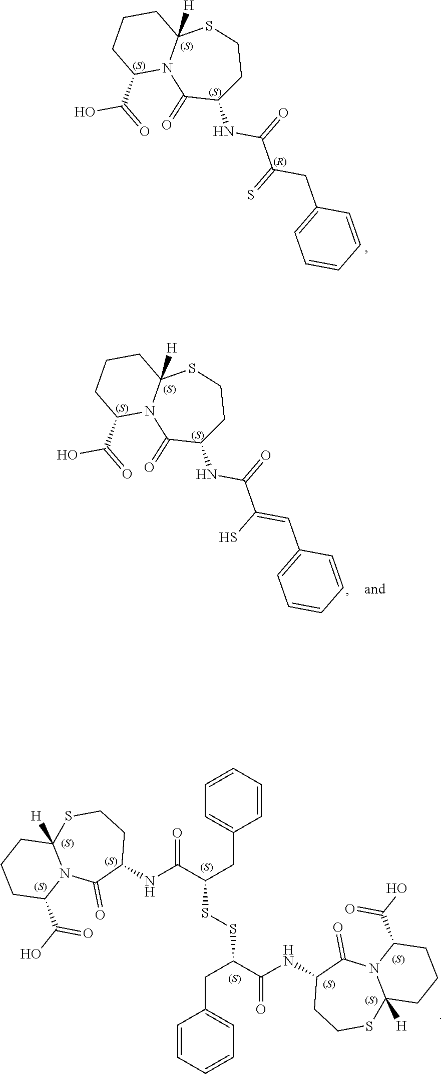

In some embodiments, the impurity or impurities can comprise one or more impurities selected from the group consisting of: a compound having a retention time of 0.72±0.02 min, a compound having a retention time of 1.51±0.02 min, a compound having a retention time of 1.53±0.02 min, a compound having a retention time of 1.6±0.2 min, a compound having a retention time of 1.9±0.2 min, a compound having a retention time of 1.93±0.2 min, a compound having a retention time of 1.96±0.2 min, a compound having a retention time of 1.97±0.2 min, a compound having a retention time of 1.99±0.2 min, and a compound having a retention time of 2.03±0.2 min, wherein the retention time is determined by chromatographic analysis at 215 nm with a Zorbax SB-C8 HPLC column. An exemplary chromatographic analysis is described in the examples as a non-chiral method for the determination of the purity of Compound 1.

In one aspect, disclosed herein is a pharmaceutical composition comprising a purified Compound 1, or a pharmaceutically acceptable salt thereof, and at least one pharmaceutically acceptable excipient, wherein: (i) the compound purity can be greater than 97.0% as determined by chromatographic analysis at 215 nm; (ii) the total amount of any one impurity can be less than 1.5% as determined by chromatographic analysis at 215 nm; (iii) the total content of all impurities can be less than 3.0% as determined by chromatographic analysis at 215 nm; or (iv) combinations thereof.

In one aspect, provided herein is a pharmaceutical composition comprising Compound 1:

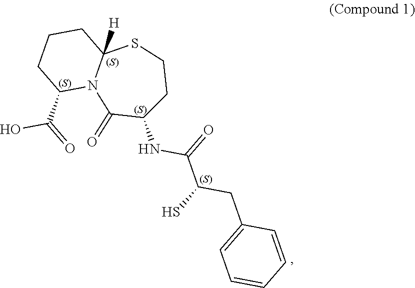

or a pharmaceutically acceptable salt thereof; and iso-Compound 1:

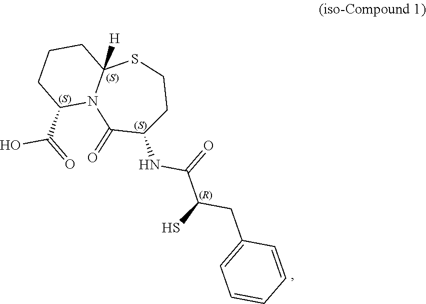

or a pharmaceutically acceptable salt thereof, and at least one pharmaceutically acceptable excipient, wherein iso-Compound 1 is present in an amount of less than 10% (w/w). In some embodiments, iso-Compound 1 is present in an amount of less than 9% (w/w). In some embodiments, iso-Compound 1 is present in an amount of less than 8% (w/w). In some embodiments, iso-Compound 1 is present in an amount of less than 7% (w/w). In some embodiments, iso-Compound 1 is present in an amount of less than 6% (w/w). In some embodiments, iso-Compound 1 is present in an amount of less than 5% (w/w). In some embodiments, iso-Compound 1 is present in an amount of less than 4% (w/w). In some embodiments, iso-Compound 1 is present in an amount of less than 3% (w/w). In some embodiments, iso-Compound 1 is present in an amount of less than 2% (w/w). In some embodiments, iso-Compound 1 is present in an amount of less than 1% (w/w). In some embodiments, iso-Compound 1 is present in an amount of less than 9% as determined by chiral HPLC. In some embodiments, iso-Compound 1 is present in an amount of less than 8% as determined by chiral HPLC. In some embodiments, iso-Compound 1 is present in an amount of less than 7% as determined by chiral HPLC. In some embodiments, iso-Compound 1 is present in an amount of less than 6% as determined by chiral HPLC. In some embodiments, iso-Compound 1 is present in an amount of less than 5% as determined by chiral HPLC. In some embodiments, iso-Compound 1 is present in an amount of less than 4% as determined by chiral HPLC. In some embodiments, iso-Compound 1 is present in an amount of less than 3% as determined by chiral HPLC. In some embodiments, iso-Compound 1 is present in an amount of less than 2% as determined by chiral HPLC. In some embodiments, iso-Compound 1 is present in an amount of less than 1% as determined by chiral HPLC.

In one aspect, provided herein is (4S,7S,10aS)-4-((S)-2-mercapto-3-phenylpropanamido)-5-oxooctahydro-7H-pyrido[2,1-b][1,3]thiazepine-7-carboxylic acid, or a pharmaceutically acceptable salt thereof. In some embodiments, (4S,7S,10aS)-4-((S)-2-mercapto-3-phenylpropanamido)-5-oxooctahydro-7H-pyrido[2,1-b][1,3]thiazepine-7-carboxylic acid is substantially free of (4S,7S,10aS)-4-((R)-2-mercapto-3-phenylpropanamido)-5-oxooctahydro-7H-pyrido[2,1-b][1,3]thiazepine-7-carboxylic acid. In some embodiments, provided herein is a pharmaceutical composition comprising (4S,7S,10aS)-4-((S)-2-mercapto-3-phenylpropanamido)-5-oxooctahydro-7H-pyrido[2,1-b][1,3]thiazepine-7-carboxylic acid, or a pharmaceutically acceptable salt thereof, and at least one pharmaceutically acceptable excipient. In some embodiments, the pharmaceutical composition comprising (4S,7S,10aS)-4-((S)-2-mercapto-3-phenylpropanamido)-5-oxooctahydro-7H-pyrido[2,1-b][1,3]thiazepine-7-carboxylic acid or a pharmaceutically acceptable salt thereof is substantially free of (4S,7S,10aS)-4-((R)-2-mercapto-3-phenylpropanamido)-5-oxooctahydro-7H-pyrido[2,1-b][1,3]thiazepine-7-carboxylic acid. In some embodiments, the (4S,7S,10aS)-4-((R)-2-mercapto-3-phenylpropanamido)-5-oxooctahydro-7H-pyrido[2,1-b][1,3]thiazepine-7-carboxylic acid is present in an amount less than 10% (w/w), less than 9% (w/w), less than 8% (w/w), less than 7% (w/w), less than 6% (w/w), less than 5% (w/w), less than 4% (w/w), less than 3% (w/w), less than 2% (w/w), or less than 1% (w/w). In some embodiments, the (4S,7S,10aS)-4-((R)-2-mercapto-3-phenylpropanamido)-5-oxooctahydro-7H-pyrido[2,1-b][1,3]thiazepine-7-carboxylic acid is present in an amount less than 10%, less than 9%, less than 8%, less than 7%, less than 6%, less than 5%, less than 4%, less than 3%, less than 2%, or less than 1% as determined by chiral HPLC. In some embodiments, the (4S,7S,10aS)-4-((S)-2-mercapto-3-phenylpropanamido)-5-oxooctahydro-7H-pyrido[2,1-b][1,3]thiazepine-7-carboxylic acid, or a pharmaceutically acceptable salt thereof, is purified, wherein (i) the compound purity is greater than 97.0% as determined by chromatographic analysis at 215 nm; (ii) the total amount of any one impurity is less than 1.5% as determined by chromatographic analysis at 215 nm; (iii) the total content of all impurities is less than 3.0% as determined by chromatographic analysis at 215 nm; or (iv) combinations thereof.

In another aspect, provided herein is a method for treating of treating a cardiac disease or disorder in a subject in need thereof, the method comprising administering Compound 1 (including purified Compound 1) as described herein. In another aspect, provided herein is Compound 1 (including purified Compound 1) as described herein for use in the treatment of a cardiac disease or disorder in a subject in need thereof. In one aspect, provided herein is use of Compound 1 (including purified Compound 1) as described herein in the manufacture of a medicament for the treatment of a cardiac disease or disorder.

In some embodiments, described herein is a process for the preparation of (4S,7S,10aS)-4-((S)-2-mercapto-3-phenylpropanamido)-5-oxooctahydro-7H-pyrido[2,1-b][1,3]thiazepine-7-carboxylic acid (Compound 1) or a salt thereof: comprising the steps of:

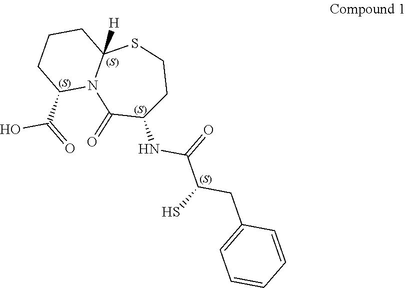

(i)(a) reacting Compound 4 or a salt thereof:

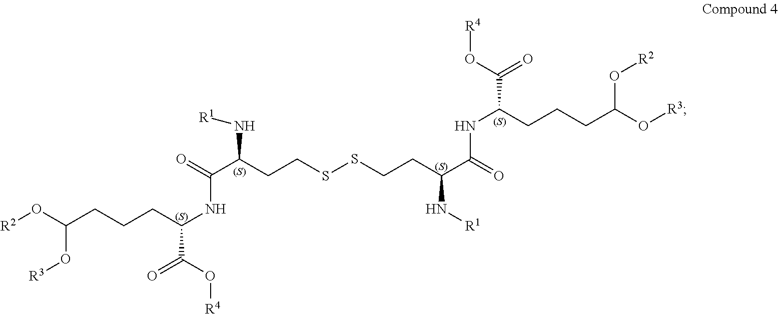

with a reagent that cleaves the disulfide bond to produce a thiol monomer, wherein
R¹ is a protecting group; and
R² and R³ are each C₁₋₃ alkyl, or R² and R³ are taken together to form dioxolane;
R⁴ is C₁₋₃ alkyl;
   (i)(b) subjecting the monomer from step (i)(a) to an acid catalyzed cyclization reaction in a suitable solvent to provide Compound 5 or a salt thereof:

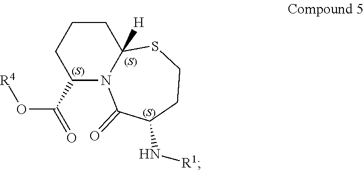

(ii) reacting Compound 5 with a suitable reagent to provide Compound 6 or a salt thereof:

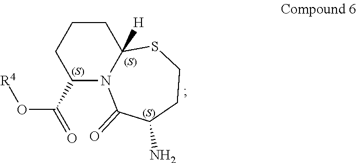

(iii) reacting Compound 6 with Compound 7 or a salt thereof:

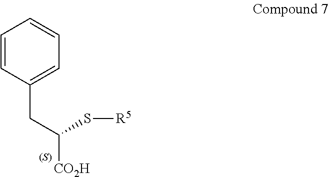

in the presence of a coupling reagent, wherein
R⁵ is —C(O)—C₁₋₃ alkyl or —C(O)-phenyl;
to provide Compound 8 or a salt thereof:

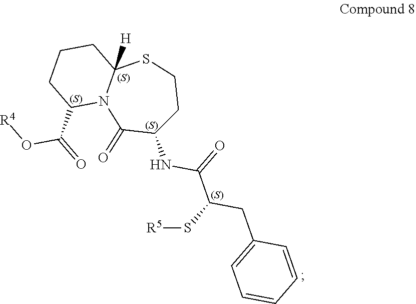

and
(iv) treating Compound 8 to provide (4S,7S,10aS)-4-((S)-2-mercapto-3-phenylpropanamido)-5-oxooctahydro-7H-pyrido[2,1-b][1,3]thiazepine-7-carboxylic acid (Compound 1) or a salt thereof. In some embodiments, R¹ is a protecting group chosen from formyl, acetyl, trifluoroacetyl, benzyl carbamate, Fmoc, Boc, phthalimide, benzyl, trityl, benzylideneamine, and tosyl. In some embodiments, R¹ is benzyl carbamate. In some embodiments, R² and R³ are each C₁₋₃ alkyl. In some embodiments, R² and R³ are taken together to form dioxolane. In some embodiments, R⁴ is methyl. In some embodiments, R⁵ is —C(O)-methyl.

In some embodiments, the reagent that cleaves the disulfide bond of step (i)(a) comprises a thiol or a phosphine. In some embodiments, the thiol is beta-mercaptoethanol, dithiothreitol, dithioerythritol, glutathione, N,N′- dimethyl-N,N′-bis(mercaptoacetyl)hydrazine, meso-2,5- dimercapto-N,N,N′,N′-tetramethyladipamide, bis(2-mercaptoethyl) sulfone, (2S)-2-amino-1,4-dimercaptobutane, 2,3-bis(mercaptomethyl)pyrazine, or 2-(dibenzylamino)butane-1,4-dithiol. In some embodiments, the reagent that cleaves the disulfide bond is dithiothreitol. In some embodiments, the phosphine is tris-(hydroxymethyl)phosphine, tris-(2-carboxyethyl)phosphine, tris(3-hydroxypropyl)phosphine, or tributyl phosphine. In some embodiments, the suitable solvent of step (i)(b) comprises water. In some embodiments, the suitable solvent of step (i)(b) comprises water and acetonitrile. In some embodiments, the water comprises less than about 50% of the solvent by volume. In some embodiments, the water comprises less than about 25% of the solvent by volume. In some embodiments, the water comprises less than about 10% of the solvent by volume. In some embodiments, the water comprises less than about 5% of the solvent by volume. In some embodiments, the acid catalyst of step (i)(b) comprises trifluoroacetic acid, chlorosulfonic acid, p-toluenesulfonic acid, methanesulfonic acid, trifluoromethanesulfonic acid, trimethylsilyl trifluoromethanesulfonate, trimethylsilyl methanesulfonate or Amberlyst. In some embodiments, the acid catalyst of step (i)(b) is trifluoroacetic acid. In some embodiments, the suitable reagent of step (ii) comprises an acid, a base, hydrogen, or an organosilicon. In some embodiments, the suitable reagent of step (ii) comprises an acid, a base, or hydrogen and a catalyst. In some embodiments, the catalyst comprises a palladium catalyst. In some embodiments, the suitable reagent of step (ii) is trimethylsilyl iodide.

In some embodiments, the coupling reagent of step (iii) facilitates the formation of an amide bond. In some embodiments, the coupling reagent of step (iii) comprises pivaloyl chloride, isobutyl chloroformate, benzotriazol-1-yloxytripyrrolidinophosphonium hexafluorophosphate (PyBOP), propylphosphonic anhydride (T3P), (benzotriazol-1-yloxy)tris(dimethylamino)phosphonium hexafluorophosphate (BOP), (1-Cyano-2-ethoxy-2-oxoethylidenaminooxy)dimethylamino-morpholino-carbenium hexafluorophosphate (COMU), 3-(diethylphosphoryloxy)-1,2,3-benzotriazin-4(3H)-one (DEPBT), O-(7-azabenzotriazol-1-yl)-N,N,N′,N′-tetramethyluronium hexafluorophosphate (HATU), O-(benzotriazol-1-yl)-N,N,N′,N′-tetramethyluronium hexafluorophosphate (HBTU), O-(6-chlorobenzotriazol-1-yl)-N,N,N′,N′-tetramethyluronium hexafluorophosphate (HCTU), O-(7-azabenzotriazol-1-yl)- N,N,N′,N′-tetramethyluronium tetrafluoroborate (TATU), O-(benzotriazol-1-yl)-N,N,N′,N′-tetramethyluronium tetrafluoroborate (TBTU), (7-azabenzotriazol-1-yloxy)tripyrrolidinophosphonium hexafluorophosphate (PyAOP), bromotripyrrolidinophosphonium hexafluorophosphate (PyBrOP), 6-chloro-benzotriazole-1-yloxy-tris-pyrrolidinophosphonium hexafluorophosphate (PyClock), chlorotripyrrolidinophosphonium hexafluorophosphate (PyCloP), bis(2-oxo-3-oxazolidinyl)phosphinic chloride (BOP—Cl), [ethyl cyano(hydroxyimino)acetato-O²]tri-1-pyrrolidinylphosphonium hexafluorophosphate (PyOxim), O-(N-succinimidyl)-1,1,3,3-tetramethyl-uronium tetrafluoroborate (TSTU), O-(5-norbornene-2,3-dicarboximido)-N,N,N′,N′-tetramethyluronium tetrafluoroborate (TNTU), O-(1,2-dihydro-2-oxo-1-pyridyl-N,N,N′,N′-tetramethyluronium tetrafluoroborate (TPTU), diisopropylcarbodiimide (DIC), carbonyldiimidazole (CDI), dicyclohexylcarbodiimide (DCC), O-[(ethoxycarbonyl)cyanomethyleneamino]-N,N,N‘N’-tetramethyluronium tetrafluoroborate (TOTU), N,N,N′,N′-tetramethyl-O-(3,4-dihydro-4-oxo-1,2,3-benzotriazin-3-yl)uronium tetrafluoroborate (TDBTU), or N,N,N′,N′-tetramethylchloroformamidinium hexafluorophosphate (TCFH) a salt of any of these, a stereoisomer of any of these, or any combination thereof. In some embodiments, the coupling reagent of step (iii) is propylphosphonic anhydride (T3P) or benzotriazol-1-yloxytripyrrolidinophosphonium hexafluorophosphate (PyBOP).

In some embodiments, wherein step (iii) further comprises purifying Compound 8 via crystallization or precipitation. In some embodiments, step (iii) further comprises purifying Compound 8 via column chromatography. In some embodiments, the treatment of step (iv) comprises step (iv)(a) cooling a solution of Compound 8 to below about 25° C. In some embodiments, the solution of Compound 8 is cooled to about 0° C. In some embodiments, the treatment of step (iv) comprises step (iv)(a) keeping a solution of Compound 8 at or below about 60° C. In some embodiments, the treatment step of (iv) further comprises step (iv)(b) contacting Compound 8 with a metal hydroxide base or a basic solution comprising a metal hydroxide. In some embodiments, the basic solution is a sodium hydroxide solution or a lithium hydroxide solution. In some embodiments, the basic solution is a sodium hydroxide solution. In some embodiments, the basic solution is a lithium hydroxide solution. In some embodiments, the treatment of step (iv) further comprises step (iv)(c) acidifying the solution to precipitate (4S,7S,10aS)-4-((S)-2-mercapto-3-phenylpropanamido)-5-oxooctahydro-7H-pyrido[2,1-b][1,3]thiazepine-7-carboxylic acid (Compound 1). In some embodiments, the solution is acidified to below about pH 3. In some embodiments, the solution is acidified to below about pH 2.5. In some embodiments, the solution is acidified with hydrochloric acid. In some embodiments, (4S,7S,10aS)-4-((S)-2-mercapto-3-phenylpropanamido)-5-oxooctahydro-7H-pyrido[2,1-b][1,3]thiazepine-7-carboxylic acid (Compound 1) is isolated via precipitation and filtration. In some embodiments, (4S,7S,10aS)-4-((S)-2-mercapto-3-phenylpropanamido)-5-oxooctahydro-7H-pyrido[2,1-b][1,3]thiazepine-7-carboxylic acid (Compound 1) is isolated via extraction. In some embodiments, Compound 1 can be purified via a trituration. In some embodiments, the trituration comprises acetonitrile. In some embodiments, the acetonitrile is heated. In some embodiments, the trituration comprises refluxing acetonitrile.

In some embodiments, the suitable solvent of step (i)(b) comprises water. In some embodiments, the water comprises less than about 50% of the solvent by volume.

In some embodiments, Compound 4:

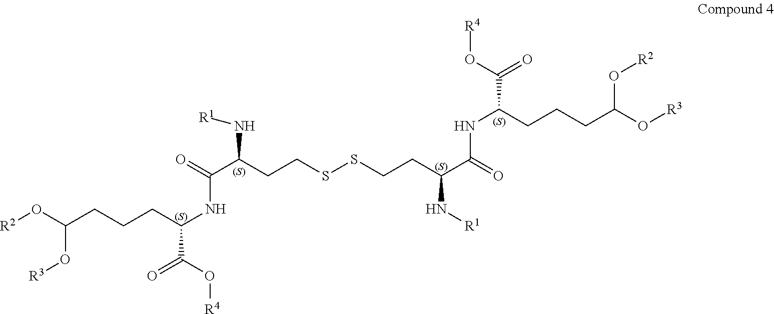

is prepared by reacting Compound 2:

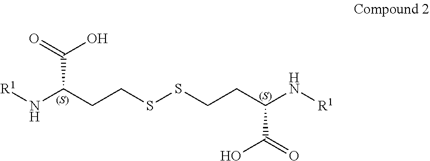

with Compound 3:

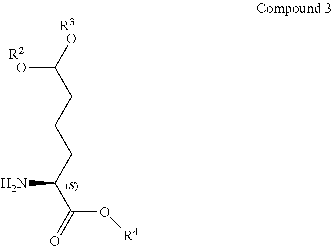

in the presence of a coupling reagent and in a suitable solvent. In some embodiments, Compound 4 can be purified by crystallization.

In some embodiments, the process further comprises purifying Compound 4 by crystallization. In some embodiments, Compound 4 is crystallized with a solvent system comprising an ether, an alcohol, an alkane (e.g., hexane, cyclohexane), acetonitrile, acetone, methyl acetate, ethyl acetate, chloroform, dichloromethane, dioxane, or an aryl hydrocarbon (e.g., toluene, benzene). In some embodiments, Compound 4 is crystallized with a solvent system comprising an ether and the ether is tetrahydrofuran, methyl-tetrahydrofuran, dioxane, methyl tert-butyl ether, diethyl ether, or dimethyl ether. In some embodiments, R¹ is a protecting group chosen from formyl, trifluoroacetyl, and benzyl carbamate. In some embodiments, R¹ is benzyl carbamate. In some embodiments, R² and R³ are each C₁₋₃alkyl. In some embodiments, R² and R³ are taken together to form dioxolane. In some embodiments, R⁴ is methyl. In some embodiments, Compound 4 is Compound 4a or Compound 4b:

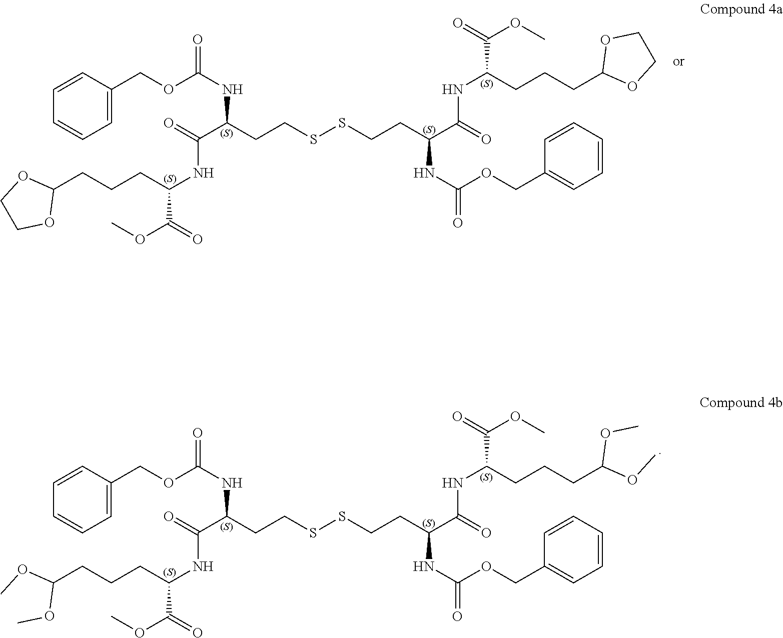

In some embodiments, Compound 4 is Compound 4a:

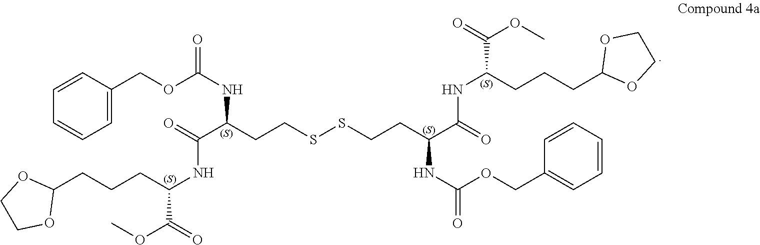

In some embodiments, Compound 3 is Compound 3a or Compound 3b:

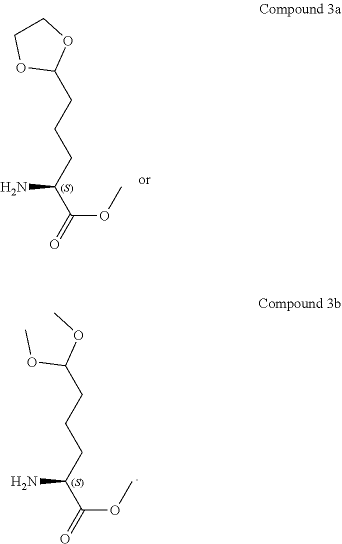

In some embodiments, Compound 3 is Compound 3a

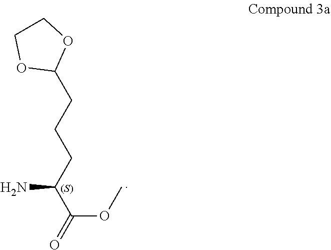

In some embodiments, the coupling reagent facilitates the formation of an amide bond. In some embodiments, the coupling reagent comprises pivaloyl chloride, isobutyl chloroformate, benzotriazol-1-yloxytripyrrolidinophosphonium hexafluorophosphate (PyBOP), propylphosphonic anhydride (T3P), (benzotriazol-1-yloxy)tris(dimethylamino)phosphonium hexafluorophosphate (BOP), (1-Cyano-2-ethoxy-2-oxoethylidenaminooxy)dimethylamino-morpholino-carbenium hexafluorophosphate (COMU), 3-(diethylphosphoryloxy)-1,2,3-benzotriazin-4(3H)-one (DEPBT), O-(7-azabenzotriazol-1-yl)-N,N,N′,N′-tetramethyluronium hexafluorophosphate (HATU), O-(benzotriazol-1-yl)-N,N,N′,N′-tetramethyluronium hexafluorophosphate (HBTU), O-(6-chlorobenzotriazol-1-yl)-N,N,N′,N′-tetramethyluronium hexafluorophosphate (HCTU), O-(7-azabenzotriazol-1-yl)- N,N,N′,N′-tetramethyluronium tetrafluoroborate (TATU), O-(benzotriazol-1-yl)- N,N,N′,N′-tetramethyluronium tetrafluoroborate (TBTU), (7-azabenzotriazol-1-yloxy)tripyrrolidinophosphonium hexafluorophosphate (PyAOP), bromotripyrrolidinophosphonium hexafluorophosphate (PyBrOP), 6-chloro-benzotriazole-1-yloxy-tris-pyrrolidinophosphonium hexafluorophosphate (PyClock), chlorotripyrrolidinophosphonium hexafluorophosphate (PyCloP), bis(2-oxo-3-oxazolidinyl)phosphinic chloride (BOP—Cl), [ethyl cyano(hydroxyimino)acetato-O²]tri-1-pyrrolidinylphosphonium hexafluorophosphate (PyOxim), O—(N-succinimidyl)-1,1,3,3-tetramethyl-uronium tetrafluoroborate (TSTU), O-(5-norbornene-2,3-dicarboximido)-N,N,N′,N′-tetramethyluronium tetrafluoroborate (TNTU), O-(1,2-dihydro-2-oxo-1-pyridyl-N,N,N′,N′-tetramethyluronium tetrafluoroborate (TPTU), diisopropylcarbodiimide (DIC), carbonyldiimidazole (CDI), dicyclohexylcarbodiimide (DCC), O-[(ethoxycarbonyl)cyanomethyleneamino]-N,N,N‘N’-tetramethyluronium tetrafluoroborate (TOTU), N,N,N′,N′-tetramethyl-O-(3,4-dihydro-4-oxo-1,2,3-benzotriazin-3-yl)uronium tetrafluoroborate (TDBTU), or N,N,N′,N′-tetramethylchloroformamidinium hexafluorophosphate (TCFH) a salt of any of these, a stereoisomer of any of these, or any combination thereof. In some embodiments, the coupling reagent comprises propylphosphonic anhydride (T3P).

In some embodiments, the suitable solvent comprises ethyl acetate, dimethylformamide, N-methyl pyrrolidone, dichloromethane, acetonitrile, tetrahydrofuran, or methyl-tetrahydrofuran. In some embodiments, the suitable solvent comprises ethyl acetate. In some embodiments, the suitable solvent comprises ethyl acetate and acetonitrile. In some embodiments, the suitable solvent comprises methyl-tetrahydrofuran, tetrahydrofuran, or a combination thereof.

In some embodiments, Compound 7 has the structure of

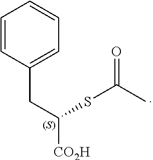

In some embodiments, the optical purity of Compound 7 is greater than about 90% enantiomeric excess. In some embodiments, the optical purity of Compound 7 is greater than about 95% enantiomeric excess. In some embodiments, the (4S,7S,10aS)-4-((S)-2-mercapto-3-phenylpropanamido)-5-oxooctahydro-7H-pyrido[2,1-b][1,3]thiazepine-7-carboxylic acid (Compound 1) is prepared with an optical purity of is greater than about 97% enantiomeric excess. In some embodiments, the (4S,7S,10aS)-4-((S)-2-mercapto-3-phenylpropanamido)-5-oxooctahydro-7H-pyrido[2,1-b][1,3]thiazepine-7-carboxylic acid (Compound 1) is prepared with an optical purity of is greater than about 98% enantiomeric excess.

In some embodiments, described herein is a process for the preparation of Compound 4a or a salt thereof:

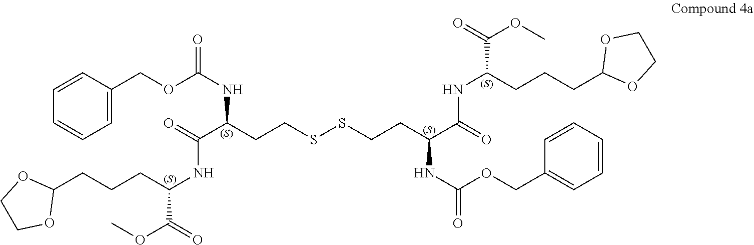

comprising reacting Compound 2-Cbz or a salt thereof:

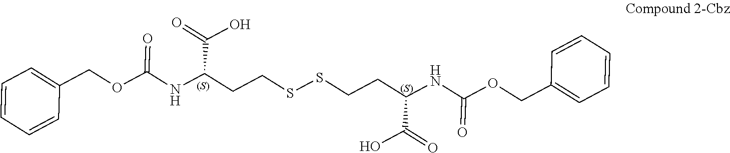

with Compound 3a or a salt thereof.

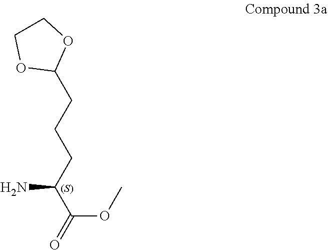

in the presence of a coupling reagent and in a suitable solvent to provide Compound 4a. In some embodiments, the process further comprises purifying Compound 4a by crystallization. In some embodiments, Compound 4a is crystallized from a solvent system comprising an ether, an alcohol, an alkane (e.g., hexane, cyclohexane), acetonitrile, acetone, methyl acetate, ethyl acetate, chloroform, dichloromethane, dioxane, or an aryl hydrocarbon (e.g., toluene, benzene). In some embodiments, Compound 4a is crystallized from a solvent system comprising an ether and the ether is tetrahydrofuran, methyl-tetrahydrofuran, dioxane, methyl tert-butyl ether, diethyl ether, or dimethyl ether.

In some embodiments, the coupling reagent facilitates the formation of an amide bond. In some embodiments, the coupling reagent comprises pivaloyl chloride, isobutyl chloroformate, benzotriazol-1-yloxytripyrrolidinophosphonium hexafluorophosphate (PyBOP), propylphosphonic anhydride (T3P), (benzotriazol-1-yloxy)tris(dimethylamino)phosphonium hexafluorophosphate (BOP), (1-Cyano-2-ethoxy-2-oxoethylidenaminooxy)dimethylamino-morpholino-carbenium hexafluorophosphate (COMU), 3-(diethylphosphoryloxy)-1,2,3-benzotriazin-4(3H)-one (DEPBT), O-(7-azabenzotriazol-1-yl)-N,N,N′,N′-tetramethyluronium hexafluorophosphate (HATU), O-(benzotriazol-1-yl)-N,N,N′,N′-tetramethyluronium hexafluorophosphate (HBTU), O-(6-chlorobenzotriazol-1-yl)-N,N,N′,N′-tetramethyluronium hexafluorophosphate (HCTU), O-(7-azabenzotriazol-1-yl)- N,N,N′,N′-tetramethyluronium tetrafluoroborate (TATU), O-(benzotriazol-1-yl)- N,N,N′,N′-tetramethyluronium tetrafluoroborate (TBTU), (7-azabenzotriazol-1-yloxy)tripyrrolidinophosphonium hexafluorophosphate (PyAOP), bromotripyrrolidinophosphonium hexafluorophosphate (PyBrOP), 6-chloro-benzotriazole-1-yloxy-tris-pyrrolidinophosphonium hexafluorophosphate (PyClock), chlorotripyrrolidinophosphonium hexafluorophosphate (PyCloP), bis(2-oxo-3-oxazolidinyl)phosphinic chloride (BOP—Cl), [ethyl cyano(hydroxyimino)acetato-O²]tri-1-pyrrolidinylphosphonium hexafluorophosphate (PyOxim), O—(N-succinimidyl)-1,1,3,3-tetramethyl-uronium tetrafluoroborate (TSTU), O-(5-norbornene-2,3-dicarboximido)-N,N,N′,N′-tetramethyluronium tetrafluoroborate (TNTU), O-(1,2-dihydro-2-oxo-1-pyridyl-N,N,N′,N′-tetramethyluronium tetrafluoroborate (TPTU), diisopropylcarbodiimide (DIC), carbonyldiimidazole (CDI), dicyclohexylcarbodiimide (DCC), O-[(ethoxycarbonyl)cyanomethyleneamino]-N,N,N‘N’-tetramethyluronium tetrafluoroborate (TOTU), N,N,N′,N′-tetramethyl-O-(3,4-dihydro-4-oxo-1,2,3-benzotriazin-3-yl)uronium tetrafluoroborate (TDBTU), or N,N,N′,N′-tetramethylchloroformamidinium hexafluorophosphate (TCFH), a salt of any of these, a stereoisomer of any of these, or any combination thereof. In some embodiments, the coupling reagent is propylphosphonic anhydride (T3P).

In some embodiments, the suitable solvent comprises ethyl acetate, dimethylformamide, N-methyl pyrrolidone, dichloromethane, acetonitrile, tetrahydrofuran, or methyl-tetrahydrofuran. In some embodiments, the suitable solvent comprises ethyl acetate. In some embodiments, the suitable solvent comprises ethyl acetate and acetonitrile.

In some embodiments, described herein is a compound with the structure:

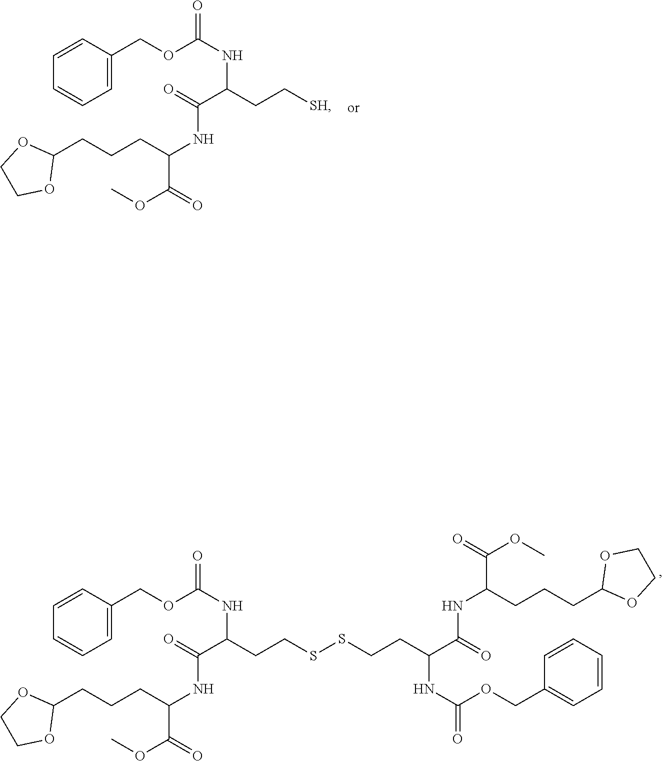 [SMILES: COC(=O)C(CCCC1OCCO1)NC(=O)C(CCS)NC(=O)OCc1ccccc1 ; COC(=O)C(CCCC1OCCO1)NC(=O)C(CCSSCCC(NC(=O)OCc1ccccc1)C(=O)NC(CCCC1OCCO1)C(=O)OC)NC(=O)OCc1ccccc1]

or a salt thereof.

In some embodiments,

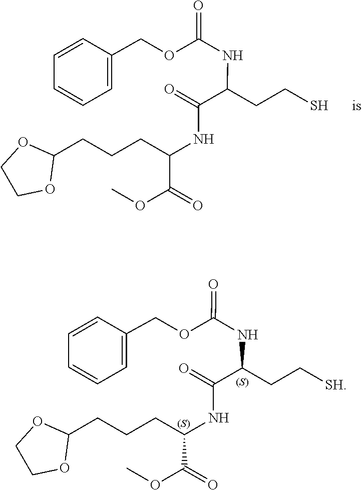

In some embodiments,

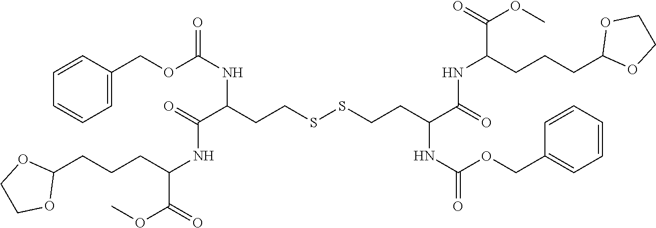 [SMILES: COC(=O)C(CCCC1OCCO1)NC(=O)C(CCSSCCC(NC(=O)OCc1ccccc1)C(=O)NC(CCCC1OCCO1)C(=O)OC)NC(=O)OCc1ccccc1]

is

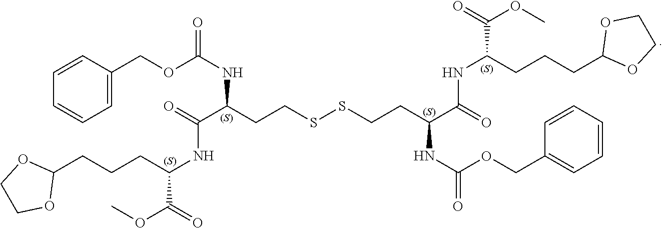

In some embodiments, described herein is a compound with the structure:

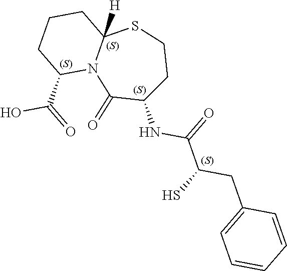

or a salt thereof,
prepared by the process described herein.

In some embodiments, described herein is a compound (4S,7S,10aS)-4-((S)-2-mercapto-3-phenylpropanamido)-5-oxooctahydro-7H-pyrido[2,1-b][1,3]thiazepine-7-carboxylic acid (Compound 1) or a salt thereof:

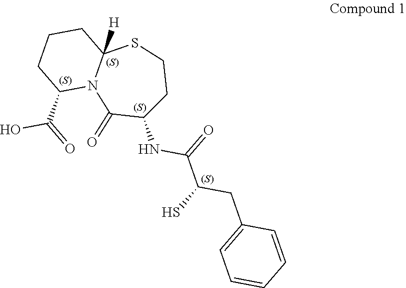

prepared by the steps of:
(i)(a) reacting Compound 4 or a salt thereof:

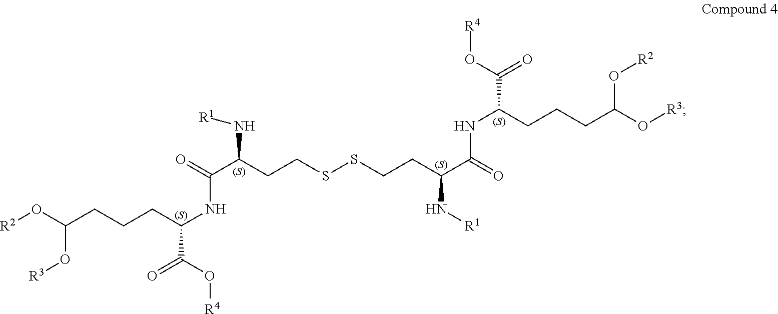

with a reagent that cleaves the disulfide bond to produce a thiol monomer, wherein
R¹ is a protecting group; and
R² and R³ are each C₁₋₃ alkyl, or R² and R³ are taken together to form dioxolane;
R⁴ is C₁₋₃ alkyl;
   (i)(b) subjecting the monomer from step (i)(a) to an acid catalyzed cyclization reaction in a suitable solvent to provide Compound 5 or a salt thereof:

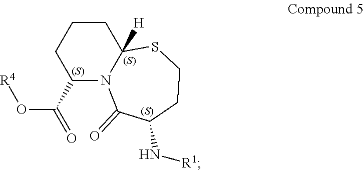

(ii) reacting Compound 5 with a suitable reagent to provide Compound 6 or a salt thereof:

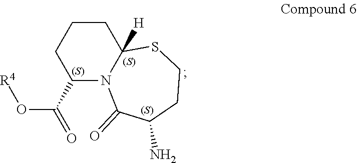

(iii) reacting Compound 6 with Compound 7 or a salt thereof:

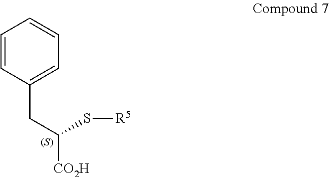

in the presence of a coupling reagent, wherein
R⁵ is —C(O)—C₁₋₃ alkyl or —C(O)-phenyl;
to provide Compound 8 or a salt thereof:

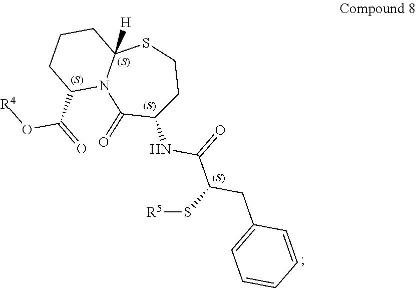

(iv) treating Compound 8 to provide (4S,7S,10aS)-4-((S)-2-mercapto-3-phenylpropanamido)-5-oxooctahydro-7H-pyrido[2,1-b][1,3]thiazepine-7-carboxylic acid (Compound 1) or a salt thereof.

In some embodiments, described herein is a process for the preparation of (4S,7S,10aS)-4-((S)-2-mercapto-3-phenylpropanamido)-5-oxooctahydro-7H-pyrido[2,1-b][1,3]thiazepine-7-carboxylic acid (Compound 1) or a salt thereof:

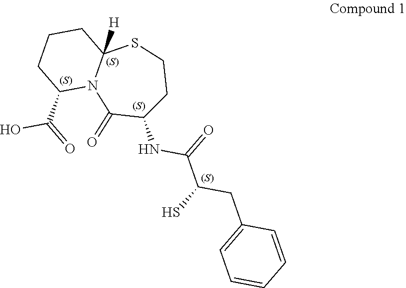

comprising the steps of:
(i)(a) reacting Compound 4a or a salt thereof:

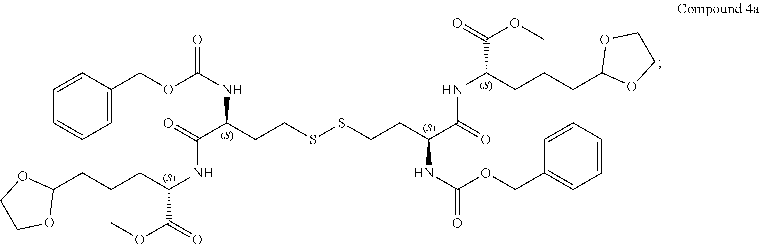

with a reagent that cleaves the disulfide bond to produce a thiol monomer;
   (i)(b) subjecting the monomer from step (i)(a) to an acid catalyzed cyclization reaction in a suitable solvent to provide Compound 5 or a salt thereof having the structure:

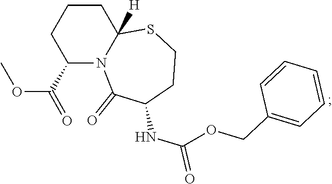

(ii) reacting Compound 5 with a suitable reagent to provide Compound 6 or a salt thereof having the structure:

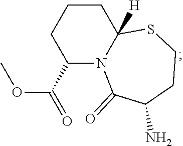

(iii) reacting Compound 6 with Compound 7 or a salt thereof having the structure:

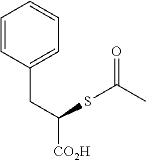

in the presence of a coupling reagent;
to provide Compound 8 or a salt thereof having a structure of:

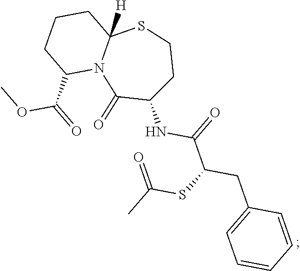

(iv) treating Compound 8 to provide (4S,7S,10aS)-4-((S)-2-mercapto-3-phenylpropanamido)-5-oxooctahydro-7H-pyrido[2,1-b][1,3]thiazepine-7-carboxylic acid (Compound 1) or a salt thereof.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### BRIEF DESCRIPTION OF THE DRAWINGS

FIG. 1A depicts the H¹NMR spectra of Compound 4a-Formyl synthesized with T3P as the coupling agent.

FIG. 1B depicts the H¹NMR spectra of Compound 4a-Formyl synthesized with pNPC as the coupling agent.

FIG. 2 depicts the H¹NMR spectra of Compound 2-Cbz.

FIG. 3 depicts the H¹NMR spectra of Compound 3a-HCl.

FIG. 4 depicts the H¹NMR spectra of Compound 4a.

FIG. 5 depicts the H¹NMR spectra of methyl (S)-2-((S)-2-(((benzyloxy)carbonyl)amino)-4-mercaptobutanamido)-5-(1,3-dioxolan-2-yl)pentanoate.

FIG. 6 depicts the H¹NMR spectra of methyl (4S,7S,10aS)-4-(((benzyloxy)carbonyl)amino)-5-oxooctahydro-7H-pyrido[2,1-b][1,3]thiazepine-7-carboxylate.

FIG. 7 depicts the H¹NMR spectra of methyl (4S,7S,10aS)-4-amino-5-oxooctahydro-7H-pyrido[2,1-b][1,3]thiazepine-7-carboxylate.

FIG. 8 depicts the H¹NMR spectra of (S)-2-(acetylthio)-3-phenylpropanoic acid.

FIG. 9 depicts the H¹NMR spectra of methyl (4S,7S,10aS)-4-((S)-2-(acetylthio)-3-phenylpropanamido)-5-oxooctahydro-7H-pyrido[2,1-b][1,3]thiazepine-7-carboxylate).

FIG. 10 depicts the H¹NMR spectra of Compound 1, (4S,7S,10aS)-4-((S)-2-mercapto-3-phenylpropanamido)-5-oxooctahydro-7H-pyrido[2,1-b][1,3]thiazepine-7-carboxylic acid.

FIG. 11A depicts chiral LC-MS signals of a mixture of crude Compound 1 containing a mixture of Compound 1 and iso-Compound 1.

FIG. 11B depicts chiral LC-MS signals of Compound 1 after purification.

FIG. 11C depicts chiral LC-MS signals of a mixture of Compound 1 and iso-Compound 1 contained in the mother liquor after purification.

FIG. 12 depicts the structure and hypothetical H¹NMR peak assignments for the proposed thioketone side product impurity listed in Table 8.

FIG. 13 depicts the structure and hypothetical H¹NMR peak assignments for the proposed unsaturated thiol side product impurity listed in Table 8.

### DETAILED DESCRIPTION

It should be understood that both the general descriptions and the detailed description below are merely illustrative and descriptive and do not limit the present technology of the present application. Those of skill in the art will recognize that there are numerous variations and modifications of this present disclosure, which are encompassed within its scope.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The headings used in the present specification are for structural purposes only and must not be construed as limiting the subject matter described.

In the present specification, the use of the singular form includes the plural form unless otherwise specified. In the present specification, the use of “or (or)” means “and/or (and/or)” unless otherwise stated. Furthermore, terms such as “element” or “component” encompass both an element and a component including one unit and an element and a component including two or more subunits unless when otherwise specified.

The recitation herein of numerical ranges by endpoints is intended to include all numbers subsumed within that range (e.g., a recitation of 1 to 5 includes 1, 1.5, 2, 2.75, 3, 3.80, 4, 4.32, and 5).

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

(4S,7S,10aS)-4-((S)-2-mercapto-3-phenylpropanamido)-5-oxooctahydro-7H-pyrido[2,1-b][1,3]thiazepine-7-carboxylic acid (Compound 1, Omapatrilat) is a vasopeptidase inhibitor with antihypertensive activity. Compound 1 exerts its effect by inhibiting angiotensin converting enzyme (ACE) and neprilysin (neutral endopeptidase or NEP). ACE inhibition results in inhibition of the renin-angiotensin-aldosterone system (RAAS) thereby leading to the reduction of vasoconstriction. Inhibition of NEP leads to reduced processing and catabolism of endogenous vasoactive peptides and peptides involved in diuresis and natriuresis, e.g. the natriuretic peptides, angiotensin I (Ang I), bradykinin (BK), and endothelin-1 (ET-1). Ultimately the dual effects of Compound 1 result in profound vasodilation and reduction of blood pressure. Therefore, Compound 1 has utility in treating cardiovascular diseases such as hypertension and heart failure.

The renin-angiotensin-aldosterone system (RAAS) is a critical regulator of blood volume and systemic vascular resistance. The three components of RAAS, renin, angiotensin II, and aldosterone, act to elevate arterial pressure in response to decreased renal blood pressure, decreased salt delivery to the distal convoluted tubule, and/or beta-agonism. Angiotensin II is converted from angiotensin I by ACE, and it has effects on the kidney, adrenal cortex, arterioles, and brain by binding to angiotensin II type I (AT1) and type II (AT2) receptors. ACE is a zinc-dependent metalloprotease found primarily in the vascular endothelium of the lungs and kidneys. ACE inhibitors (e.g., Compound 1) chelate the zinc essential for ACE activity and decrease the production of angiotensin II, and can be used for treating cardiovascular diseases such as hypertension, heart failure, and acute myocardial infarction. ACE inhibitors (e.g., Compound 1) can also be used for a variety of kidney diseases, such as diabetic nephropathy, kidney transplant, and proteinuric chronic kidney disease.

The natriuretic peptide (NP) system consists primarily of three well-characterized NP, including atrial natriuretic peptide (ANP) and B-type natriuretic peptide (BNP) from cardiomyocytes, and C-type natriuretic peptide (CNP) from endothelial and renal cells. All three NPs function via the second messenger cGMP, and exhibit cardiorenal protective properties including natriuresis, vasodilation, inhibition of RAAS, positive lusitropism, and inhibition of fibrosis. NEP is a zinc-dependent metalloprotease critical for the processing and catabolism of vasoactive peptides and peptides involved in diuresis and natriuresis, e.g. the NPs, angiotensin I (Ang I), bradykinin (BK), and endothelin-1 (ET-1). NEP inhibition can increase endogenous NP levels, which is beneficial to lowing blood pressure in hypertension. NEP inhibition can also increase Ang I, which is converted by ACE to angiotensin II, and this can lead to vasoconstriction. The ACE inhibitor activity of Compound 1 attenuates vasoconstriction caused by NEP inhibition, and thus the combination can decrease systemic and renal vascular resistance, suppress aldosterone, and increase natriuresis and diuresis. Thus through its dual action, Compound 1 can be used to treat cardiovascular and kidney diseases.

Compounds described herein can be synthesized using standard synthetic techniques such as those described in, for example, March's Advanced Organic Chemistry, 6th Edition, John Wiley and Sons, Inc. Alternative conditions for the synthetic transformations described herein may be employed such as variation of solvent, reaction temperature, reaction time, as well as different chemical reagents and other reaction conditions.

### Definitions

As used in the specification and appended claims, unless specified to the contrary, the following terms have the meaning indicated below. The section headings used herein are for organizational purposes only and are not to be construed as limiting the subject matter described.

As used herein and in the appended claims, the singular forms “a,” “an,” and “the” include plural referents unless the context clearly dictates otherwise. Thus, for example, reference to “an agent” includes a plurality of such agents. When ranges are used herein for physical properties, such as molecular weight, or chemical properties, such as chemical formulae, all combinations and subcombinations of ranges and specific embodiments therein are intended to be included.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value.

The term “comprising” (and related terms such as “comprise” or “comprises” or “having” or “including”) is open ended and does not exclude the presence of additional unrecited elements.

“Alkyl” refers to an optionally substituted straight-chain, or optionally substituted branched-chain saturated hydrocarbon monoradical. An alkyl group can have from one to about twenty carbon atoms, from one to about ten carbon atoms, or from one to six carbon atoms. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl, and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl can be a C₁-C₁₀ alkyl, a C₁-C₉ alkyl, a C₁-C₅ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group can be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl can be optionally substituted with oxo, halogen, —CN, —CF₃, —OH, —OMe, —NH₂, —NO₂, or —C≡CH. In some embodiments, the alkyl can be optionally substituted with oxo, halogen, —CN, —CF₃, —OH, or —OMe. In some embodiments, the alkyl can be optionally substituted with halogen.

As used herein, C₁-Cₓ (or C₁₋ₓ) includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl. Also, by way of example, C₀-C₂ alkylene includes a direct bond, —CH₂—, and —CH₂CH₂— linkages.

“Coupling reagent” as used herein refers to one or more reagents that facilitate the formation of an amide or peptide bond. Examples include, but are not limited to, carbodiimides, aminium salts, uranium salts, and phosphonium salts.

The term “protecting group” as used herein refers to a reversibly formed derivative of an existing functional group in a molecule or compound. The protecting group is temporarily attached to decrease reactivity so that the protected functional group does not react under synthetic conditions to which the molecule or compound is subjected in one or more subsequent steps.

The term “substantially free” as used herein refers to a compound which, under chromatographic conditions, is not shown to have any significant amount of detectable contaminants. In some embodiments, levels of the contaminant are less than 1%. In some embodiments, levels of the contaminant are less than 0.75% In some embodiments, levels of the contaminant are less than 0.5%.

The term “suitable reagent”, “acceptable reagent”, or “appropriate reagent” as used herein refer to reagents capable of facilitating the depicted chemical reaction. A suitable reagent or acceptable reagent can include one or more individual reagents. For example, a suitable reagent can include a single reagent, a mixture of reagents, or a combination of reagents. A suitable reagent can be used in the depicted reaction and added in one portion, or one or more suitable reagents can be used in the depicted reaction and added at different points in time.

The term “suitable solvent”, “acceptable solvent”, or “appropriate solvent” as used herein refer to solvents capable of facilitating the depicted chemical reaction. A suitable solvent or acceptable solvent can include one or more individual solvents. For example, a suitable solvent can include a single solvent, a mixture of solvents, or a combination of solvents.

The term “pharmaceutically acceptable,” as used herein, generally refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt,” as used herein, generally refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D.C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviours. Also, because the salt-forming molecule can be in equilibrium with a neutral form, passage through biological membranes can be adjusted.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

Certain compounds described herein may exist in tautomeric forms, and all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

### Compound 1

Compound 1 refers to 2(4S,7S,10aS)-4-((S)-2-mercapto-3-phenylpropanamido)-5-oxooctahydro-7H-pyrido[2,1-b][1,3]thiazepine-7-carboxylic acid, which has the chemical structure shown below.

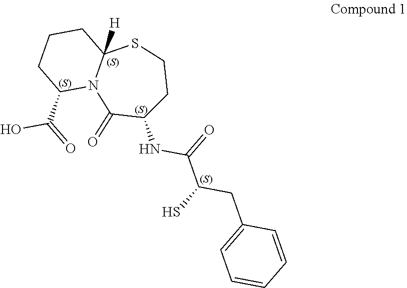

### Purified Compound 1

In one aspect, disclosed herein is a purified compound having the structure of (4S,7S,10aS)-4-((S)-2-mercapto-3-phenylpropanamido)-5-oxooctahydro-7H-pyrido[2,1-b][1,3]thiazepine-7-carboxylic acid (Compound 1) or a pharmaceutically acceptable salt thereof:

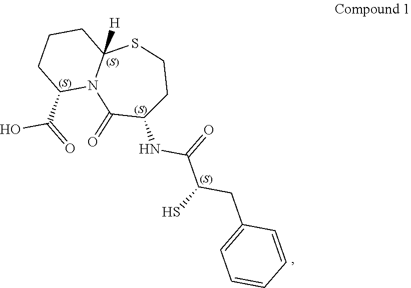

wherein
(i) the compound purity is greater than 97.0% as determined by chromatographic analysis at 215 nm;
(ii) the total amount of any one impurity is less than 1.5% as determined by chromatographic analysis at 215 nm;
(iii) the total content of all impurities is less than 3.0% as determined by chromatographic analysis at 215 nm; or
(iv) combinations thereof.

In some embodiments, purity of Compound 1 can be greater than 97% as determined by chromatographic analysis at 215 nm. In some embodiments, purity of Compound 1 can be greater than 98% as determined by chromatographic analysis at 215 nm. In some embodiments, purity of Compound 1 can be greater than 99% as determined by chromatographic analysis at 215 nm.

In some embodiments, the total amount of any one impurity in Compound 1 can be less than 1.5% as determined by chromatographic analysis at 215 nm. In some embodiments, the total amount of any one impurity in Compound 1 can be less than 0.75% as determined by chromatographic analysis at 215 nm.

In some embodiments, the total content of all impurities in Compound 1 can be less than 3.0% as determined by chromatographic analysis at 215 nm. In some embodiments, the total content of all impurities in Compound 1 can be less than 2.0% as determined by chromatographic analysis at 215 nm.

In some embodiments, Compound 1 can be substantially free of

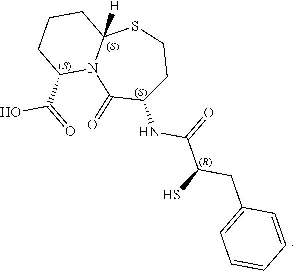

In some embodiments,

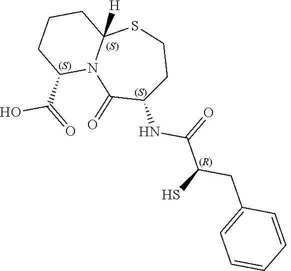

is present in an amount less than 10% as determined by chiral HPLC. In some embodiments,

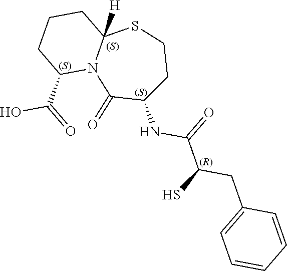

is present in an amount less than 8% as determined by chiral HPLC. In some embodiments,

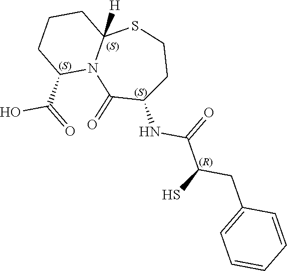

is present in an amount less than 6% as determined by chiral HPLC. In some embodiments,

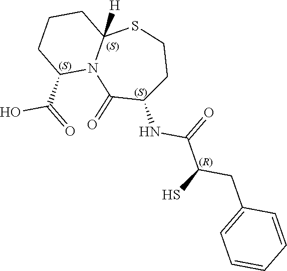

is present in an amount less than 4% as determined by chiral HPLC. In some embodiments,

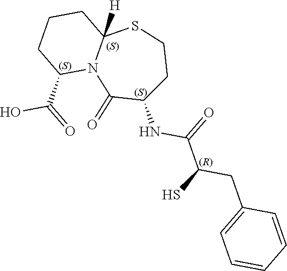

is present in an amount less than 2% as determined by chiral HPLC. In some embodiments,

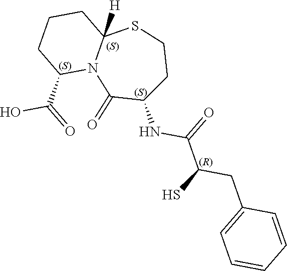

is present in an amount less than 1% as determined by chiral HPLC. In some embodiments, Compound 1 can have an optical purity of greater than about 97% enantiomeric excess. In some embodiments, Compound 1 can have an optical purity of greater than about 98% enantiomeric excess. In some embodiments, Compound 1 can have an optical purity of greater than about 99% enantiomeric excess.

In some embodiments, the impurity or impurities in Compound 1 can comprise one or more of the impurities selected from the group consisting of

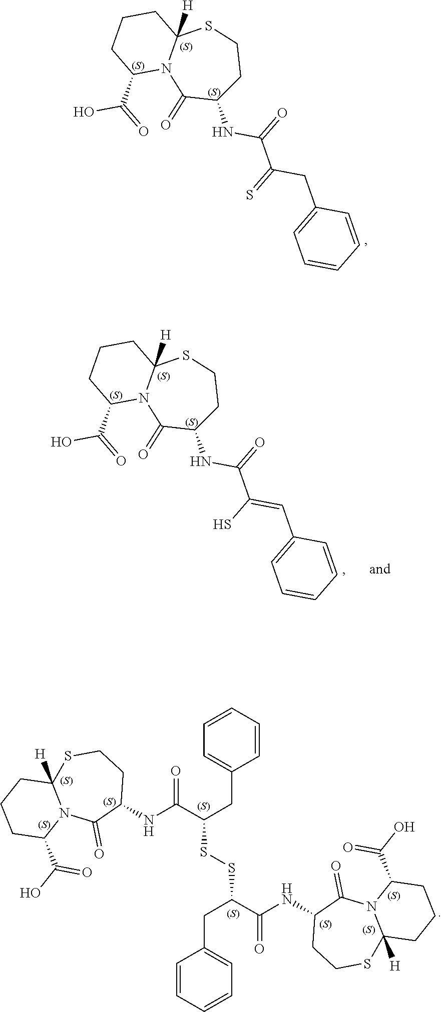

In some embodiments, the impurity can be

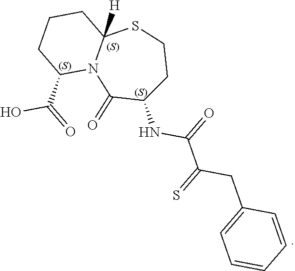

In some embodiments, the impurity can be

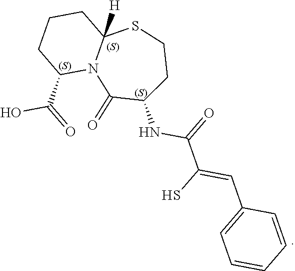

In some embodiments, the impurity can be

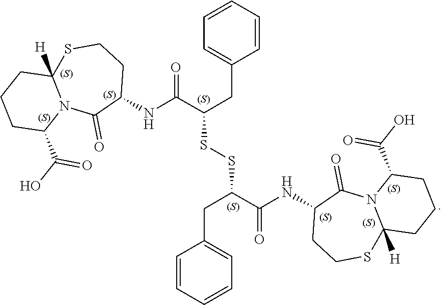

In some embodiments, the impurity or impurities in Compound 1 can comprise one or more impurities selected from the group consisting of: a compound having a retention time of 0.72±0.02 min, a compound having a retention time of 1.51±0.02 min, a compound having a retention time of 1.53±0.02 min, a compound having a retention time of 1.6±0.2 min, a compound having a retention time of 1.9±0.2 min, a compound having a retention time of 1.93±0.2 min, a compound having a retention time of 1.96±0.2 min, a compound having a retention time of 1.97±0.2 min, a compound having a retention time of 1.99±0.2 min, and a compound having a retention time of 2.03±0.2 min, wherein the retention time is determined by chromatographic analysis at 215 nm. In some embodiments, the chromatographic analysis is performed with a Zorbax SB-C8 HPLC column. An exemplary chromatographic analysis is described in the examples as a non-chiral method for the determination of the purity of Compound 1. In some embodiments, the impurity or impurities comprises two or more impurities selected from the group consisting of: a compound having a retention time of 0.72±0.02 min, a compound having a retention time of 1.51±0.02 min, a compound having a retention time of 1.53±0.02 min, a compound having a retention time of 1.6±0.2 min, a compound having a retention time of 1.9±0.2 min, a compound having a retention time of 1.93±0.2 min, a compound having a retention time of 1.96±0.2 min, a compound having a retention time of 1.97±0.2 min, a compound having a retention time of 1.99±0.2 min, and a compound having a retention time of 2.03±0.2 min. In some embodiments, the impurity or impurities comprises three or more impurities selected from the group consisting of: a compound having a retention time of 0.72±0.02 min, a compound having a retention time of 1.51±0.02 min, a compound having a retention time of 1.53±0.02 min, a compound having a retention time of 1.6±0.2 min, a compound having a retention time of 1.9±0.2 min, a compound having a retention time of 1.93±0.2 min, a compound having a retention time of 1.96±0.2 min, a compound having a retention time of 1.97±0.2 min, a compound having a retention time of 1.99±0.2 min, and a compound having a retention time of 2.03±0.2 min. In some embodiments, the impurity or impurities comprises four or more impurities selected from the group consisting of: a compound having a retention time of 0.72±0.02 min, a compound having a retention time of 1.51±0.02 min, a compound having a retention time of 1.53±0.02 min, a compound having a retention time of 1.6±0.2 min, a compound having a retention time of 1.9±0.2 min, a compound having a retention time of 1.93±0.2 min, a compound having a retention time of 1.96±0.2 min, a compound having a retention time of 1.97±0.2 min, a compound having a retention time of 1.99±0.2 min, and a compound having a retention time of 2.03±0.2 min. In some embodiments, the impurity or impurities comprises five or more impurities selected from the group consisting of: a compound having a retention time of 0.72±0.02 min, a compound having a retention time of 1.51±0.02 min, a compound having a retention time of 1.53±0.02 min, a compound having a retention time of 1.6±0.2 min, a compound having a retention time of 1.9±0.2 min, a compound having a retention time of 1.93±0.2 min, a compound having a retention time of 1.96±0.2 min, a compound having a retention time of 1.97±0.2 min, a compound having a retention time of 1.99±0.2 min, and a compound having a retention time of 2.03±0.2 min. In some embodiments, the impurity or impurities comprises six or more impurities selected from the group consisting of: a compound having a retention time of 0.72±0.02 min, a compound having a retention time of 1.51±0.02 min, a compound having a retention time of 1.53±0.02 min, a compound having a retention time of 1.6±0.2 min, a compound having a retention time of 1.9±0.2 min, a compound having a retention time of 1.93±0.2 min, a compound having a retention time of 1.96±0.2 min, a compound having a retention time of 1.97±0.2 min, a compound having a retention time of 1.99±0.2 min, and a compound having a retention time of 2.03±0.2 min. In some embodiments, the impurity or impurities comprises seven or more impurities selected from the group consisting of: a compound having a retention time of 0.72±0.02 min, a compound having a retention time of 1.51±0.02 min, a compound having a retention time of 1.53±0.02 min, a compound having a retention time of 1.6±0.2 min, a compound having a retention time of 1.9±0.2 min, a compound having a retention time of 1.93±0.2 min, a compound having a retention time of 1.96±0.2 min, a compound having a retention time of 1.97±0.2 min, a compound having a retention time of 1.99±0.2 min, and a compound having a retention time of 2.03±0.2 min. In some embodiments, the impurity or impurities comprises eight or more impurities selected from the group consisting of: a compound having a retention time of 0.72±0.02 min, a compound having a retention time of 1.51±0.02 min, a compound having a retention time of 1.53±0.02 min, a compound having a retention time of 1.6±0.2 min, a compound having a retention time of 1.9±0.2 min, a compound having a retention time of 1.93±0.2 min, a compound having a retention time of 1.96±0.2 min, a compound having a retention time of 1.97±0.2 min, a compound having a retention time of 1.99±0.2 min, and a compound having a retention time of 2.03±0.2 min. In some embodiments, the impurity or impurities comprises nine or more impurities selected from the group consisting of: a compound having a retention time of 0.72±0.02 min, a compound having a retention time of 1.51±0.02 min, a compound having a retention time of 1.53±0.02 min, a compound having a retention time of 1.6±0.2 min, a compound having a retention time of 1.9±0.2 min, a compound having a retention time of 1.93±0.2 min, a compound having a retention time of 1.96±0.2 min, a compound having a retention time of 1.97±0.2 min, a compound having a retention time of 1.99±0.2 min, and a compound having a retention time of 2.03±0.2 min. In some embodiments, the impurity or impurities comprises eight or more impurities selected from the group consisting of: a compound having a retention time of 0.72±0.02 min, a compound having a retention time of 1.51±0.02 min, a compound having a retention time of 1.53±0.02 min, a compound having a retention time of 1.6±0.2 min, a compound having a retention time of 1.9±0.2 min, a compound having a retention time of 1.93±0.2 min, a compound having a retention time of 1.96±0.2 min, a compound having a retention time of 1.97±0.2 min, a compound having a retention time of 1.99±0.2 min, and a compound having a retention time of 2.03±0.2 min. In some embodiments, the impurity or impurities comprises seven or fewer impurities selected from the group consisting of: a compound having a retention time of 0.72±0.02 min, a compound having a retention time of 1.51±0.02 min, a compound having a retention time of 1.53±0.02 min, a compound having a retention time of 1.6±0.2 min, a compound having a retention time of 1.9±0.2 min, a compound having a retention time of 1.93±0.2 min, a compound having a retention time of 1.96±0.2 min, a compound having a retention time of 1.97±0.2 min, a compound having a retention time of 1.99±0.2 min, and a compound having a retention time of 2.03±0.2 min. In some embodiments, the impurity or impurities comprises six or fewer impurities selected from the group consisting of: a compound having a retention time of 0.72±0.02 min, a compound having a retention time of 1.51±0.02 min, a compound having a retention time of 1.53±0.02 min, a compound having a retention time of 1.6±0.2 min, a compound having a retention time of 1.9±0.2 min, a compound having a retention time of 1.93±0.2 min, a compound having a retention time of 1.96±0.2 min, a compound having a retention time of 1.97±0.2 min, a compound having a retention time of 1.99±0.2 min, and a compound having a retention time of 2.03±0.2 min. In some embodiments, the impurity or impurities comprises five or fewer impurities selected from the group consisting of: a compound having a retention time of 0.72±0.02 min, a compound having a retention time of 1.51±0.02 min, a compound having a retention time of 1.53±0.02 min, a compound having a retention time of 1.6±0.2 min, a compound having a retention time of 1.9±0.2 min, a compound having a retention time of 1.93±0.2 min, a compound having a retention time of 1.96±0.2 min, a compound having a retention time of 1.97±0.2 min, a compound having a retention time of 1.99±0.2 min, and a compound having a retention time of 2.03±0.2 min. In some embodiments, the impurity or impurities comprises four or fewer impurities selected from the group consisting of: a compound having a retention time of 0.72±0.02 min, a compound having a retention time of 1.51±0.02 min, a compound having a retention time of 1.53±0.02 min, a compound having a retention time of 1.6±0.2 min, a compound having a retention time of 1.9±0.2 min, a compound having a retention time of 1.93±0.2 min, a compound having a retention time of 1.96±0.2 min, a compound having a retention time of 1.97±0.2 min, a compound having a retention time of 1.99±0.2 min, and a compound having a retention time of 2.03±0.2 min. In some embodiments, the impurity or impurities comprises three or fewer impurities selected from the group consisting of: a compound having a retention time of 0.72±0.02 min, a compound having a retention time of 1.51±0.02 min, a compound having a retention time of 1.53±0.02 min, a compound having a retention time of 1.6±0.2 min, a compound having a retention time of 1.9±0.2 min, a compound having a retention time of 1.93±0.2 min, a compound having a retention time of 1.96±0.2 min, a compound having a retention time of 1.97±0.2 min, a compound having a retention time of 1.99±0.2 min, and a compound having a retention time of 2.03±0.2 min. In some embodiments, the impurity or impurities comprises two or fewer impurities selected from the group consisting of: a compound having a retention time of 0.72±0.02 min, a compound having a retention time of 1.51±0.02 min, a compound having a retention time of 1.53±0.02 min, a compound having a retention time of 1.6±0.2 min, a compound having a retention time of 1.9±0.2 min, a compound having a retention time of 1.93±0.2 min, a compound having a retention time of 1.96±0.2 min, a compound having a retention time of 1.97±0.2 min, a compound having a retention time of 1.99±0.2 min, and a compound having a retention time of 2.03±0.2 min.

(4S,7S,10aS)-4-((S)-2-mercapto-3-phenylpropanamido)-5-oxooctahydro-7H-pyrido[2,1-b][1,3]thiazepine-7-carboxylic acid (Compound 1) binds tightly to the NEP and ACE proteins, forming close connections with active site zinc and the S₁, S₁′, and S₂′ pockets. In particular, the thiol group coordinates to the zinc ion as well as two water molecules in the active site, and the phenyl group extends deeply into the S₁′ pocket where it forms extensive hydrophobic interactions (see Sharma et al, “Molecular Basis for Omapatrilat and Sampatrilat Binding to Neprilysin- Implications for Dual Inhibitor Design with Angiotensin-Converting Enzyme” J. Med. Chem. 2020. 63, p 5488-5500). Importantly, the interactions with active site zinc and the S₁′ pocket require Compound 1 to be in a specific orientation. Altering the stereochemistry of the compound will change the binding interaction with NEP and ACE, leading to decreased potency. In some embodiments, (4S,7S,10aS)-4-((S)-2-mercapto-3-phenylpropanamido)-5-oxooctahydro-7H-pyrido[2,1-b][1,3]thiazepine-7-carboxylic acid (Compound 1) provided herein is substantially free of (4S,7S,10aS)-4-((R)-2-mercapto-3-phenylpropanamido)-5-oxooctahydro-7H-pyrido[2,1-b][1,3]thiazepine-7-carboxylic acid (iso-Compound 1) and has improved activity relative to epimeric mixtures containing increased amounts of iso-Compound 1.

In some embodiments, provided herein is (4S,7S,10aS)-4-((S)-2-mercapto-3-phenylpropanamido)-5-oxooctahydro-7H-pyrido[2,1-b][1,3]thiazepine-7-carboxylic acid, or a pharmaceutically acceptable salt thereof. In some embodiments, (4S,7S,10aS)-4-((S)-2-mercapto-3-phenylpropanamido)-5-oxooctahydro-7H-pyrido[2,1-b][1,3]thiazepine-7-carboxylic acid is substantially free of (4S,7S,10aS)-4-((R)-2-mercapto-3-phenylpropanamido)-5-oxooctahydro-7H-pyrido[2,1-b][1,3]thiazepine-7-carboxylic acid. In some embodiments, provided herein is a pharmaceutical composition comprising (4S,7S,10aS)-4-((S)-2-mercapto-3-phenylpropanamido)-5-oxooctahydro-7H-pyrido[2,1-b][1,3]thiazepine-7-carboxylic acid, or a pharmaceutically acceptable salt thereof, and a pharmaceutically acceptable excipient. In some embodiments, the pharmaceutical composition comprising (4S,7S,10aS)-4-((S)-2-mercapto-3-phenylpropanamido)-5-oxooctahydro-7H-pyrido[2,1-b][1,3]thiazepine-7-carboxylic acid or a pharmaceutically acceptable salt thereof is substantially free of (4S,7S,10aS)-4-((R)-2-mercapto-3-phenylpropanamido)-5-oxooctahydro-7H-pyrido[2,1-b][1,3]thiazepine-7-carboxylic acid. In some embodiments, the (4S,7S,10aS)-4-((R)-2-mercapto-3-phenylpropanamido)-5-oxooctahydro-7H-pyrido[2,1-b][1,3]thiazepine-7-carboxylic acid is present in an amount less than 10% (w/w), less than 9% (w/w), less than 8% (w/w), less than 7% (w/w), less than 6% (w/w), less than 5% (w/w), less than 4% (w/w), less than 3% (w/w), less than 2% (w/w), or less than 1% (w/w). In some embodiments, the pharmaceutical composition comprising (4S,7S,10aS)-4-((R)-2-mercapto-3-phenylpropanamido)-5-oxooctahydro-7H-pyrido[2,1-b][1,3]thiazepine-7-carboxylic acid is present in an amount less than 10%, less than 9%, less than 8%, less than 7%, less than 6%, less than 5%, less than 4%, less than 3%, less than 2%, or less than 1% as determined by chiral HPLC. In some embodiments, the (4S,7S,10aS)-4-((R)-2-mercapto-3-phenylpropanamido)-5-oxooctahydro-7H-pyrido[2,1-b][1,3]thiazepine-7-carboxylic acid is present in an amount less than 10% (w/w). In some embodiments, the (4S,7S,10aS)-4-((R)-2-mercapto-3-phenylpropanamido)-5-oxooctahydro-7H-pyrido[2,1-b][1,3]thiazepine-7-carboxylic acid is present in an amount less than 9% (w/w). In some embodiments, the (4S,7S,10aS)-4-((R)-2-mercapto-3-phenylpropanamido)-5-oxooctahydro-7H-pyrido[2,1-b][1,3]thiazepine-7-carboxylic acid is present in an amount less than 8% (w/w). In some embodiments, the (4S,7S,10aS)-4-((R)-2-mercapto-3-phenylpropanamido)-5-oxooctahydro-7H-pyrido[2,1-b][1,3]thiazepine-7-carboxylic acid is present in an amount less than 7% (w/w). In some embodiments, the (4S,7S,10aS)-4-((R)-2-mercapto-3-phenylpropanamido)-5-oxooctahydro-7H-pyrido[2,1-b][1,3]thiazepine-7-carboxylic acid is present in an amount less than 6% (w/w). In some embodiments, the (4S,7S,10aS)-4-((R)-2-mercapto-3-phenylpropanamido)-5-oxooctahydro-7H-pyrido[2,1-b][1,3]thiazepine-7-carboxylic acid is present in an amount less than 5% (w/w). In some embodiments, the (4S,7S,10aS)-4-((R)-2-mercapto-3-phenylpropanamido)-5-oxooctahydro-7H-pyrido[2,1-b][1,3]thiazepine-7-carboxylic acid is present in an amount less than 4% (w/w). In some embodiments, the (4S,7S,10aS)-4-((R)-2-mercapto-3-phenylpropanamido)-5-oxooctahydro-7H-pyrido[2,1-b][1,3]thiazepine-7-carboxylic acid is present in an amount less than 3% (w/w). In some embodiments, the (4S,7S,10aS)-4-((R)-2-mercapto-3-phenylpropanamido)-5-oxooctahydro-7H-pyrido[2,1-b][1,3]thiazepine-7-carboxylic acid is present in an amount less than less than 2% (w/w). In some embodiments, the (4S,7S,10aS)-4-((R)-2-mercapto-3-phenylpropanamido)-5-oxooctahydro-7H-pyrido[2,1-b][1,3]thiazepine-7-carboxylic acid is present in an amount less than 1% (w/w). In some embodiments, the (4S,7S,10aS)-4-((S)-2-mercapto-3-phenylpropanamido)-5-oxooctahydro-7H-pyrido[2,1-b][1,3]thiazepine-7-carboxylic acid, or a pharmaceutically acceptable salt thereof, is purified, wherein (i) the compound purity is greater than 97.0% as determined by chromatographic analysis at 215 nm; (ii) the total amount of any one impurity is less than 1.5% as determined by chromatographic analysis at 215 nm; (iii) the total content of all impurities is less than 3.0% as determined by chromatographic analysis at 215 nm; or (iv) combinations thereof.

In one aspect, the disclosure provides a method of treating a cardiac disease or disorder in a subject in need thereof, the method comprising administering to the subject a purified compound having the structure of (4S,7S,10aS)-4-((S)-2-mercapto-3-phenylpropanamido)-5-oxooctahydro-7H-pyrido[2,1-b][1,3]thiazepine-7-carboxylic acid (Compound 1) or a pharmaceutically acceptable salt thereof:

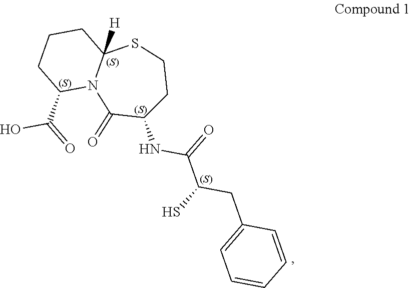

wherein
(i) the compound purity is greater than 97.0% as determined by chromatographic analysis at 215 nm;
(ii) the total amount of any one impurity is less than 1.5% as determined by chromatographic analysis at 215 nm;
(iii) the total content of all impurities is less than 3.0% as determined by chromatographic analysis at 215 nm; or
(iv) combinations thereof.

In some embodiments, (4S,7S,10aS)-4-((S)-2-mercapto-3-phenylpropanamido)-5-oxooctahydro-7H-pyrido[2,1-b][1,3]thiazepine-7-carboxylic acid is substantially free of (4S,7S,10aS)-4-((R)-2-mercapto-3-phenylpropanamido)-5-oxooctahydro-7H-pyrido[2,1-b][1,3]thiazepine-7-carboxylic acid. In some embodiments, the (4S,7S,10aS)-4-((R)-2-mercapto-3-phenylpropanamido)-5-oxooctahydro-7H-pyrido[2,1-b][1,3]thiazepine-7-carboxylic acid is present in an amount less than 10% (w/w) or less than 10% as determined by chiral HPLC. In some embodiments, the (4S,7S,10aS)-4-((R)-2-mercapto-3-phenylpropanamido)-5-oxooctahydro-7H-pyrido[2,1-b][1,3]thiazepine-7-carboxylic acid is present in an amount less than 1% (w/w) or less than 1% as determined by chiral HPLC.

### Preparation of Compound 1

Described herein are methods for the synthesis of Compound 1 as outlined in Scheme A.

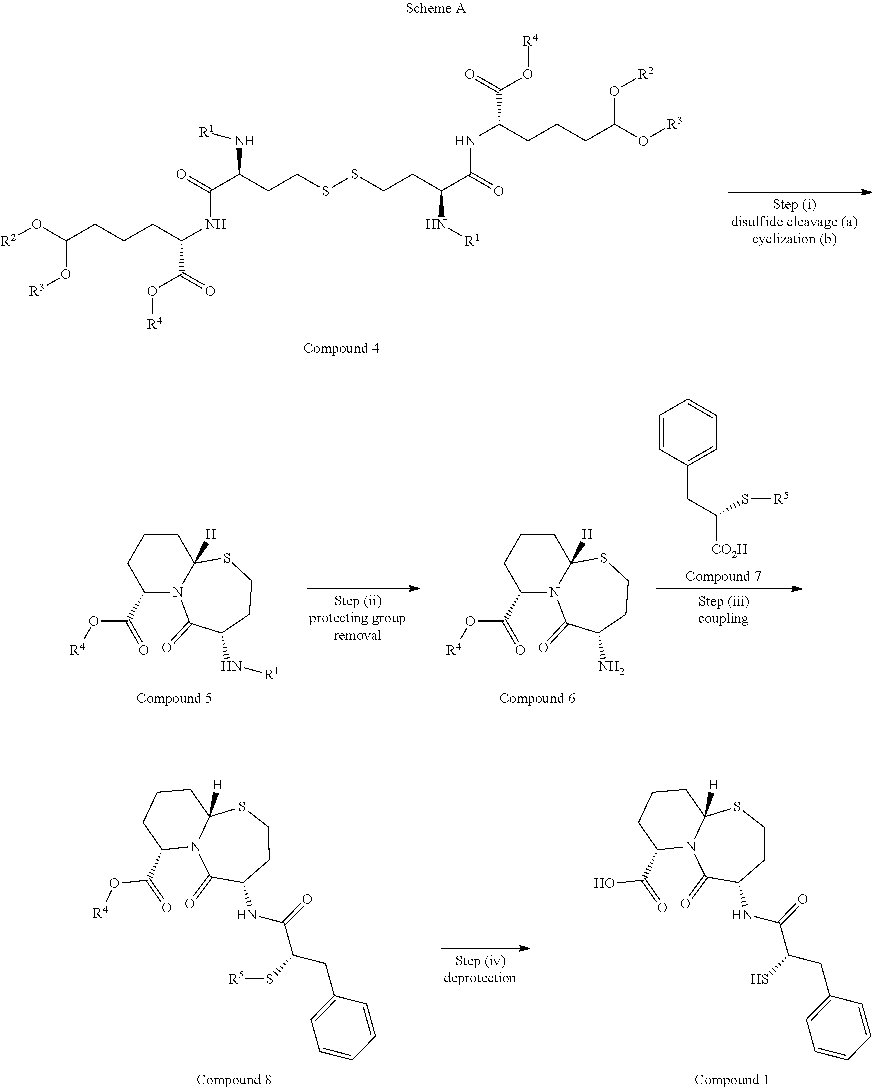

where R¹ can be a protecting group;
R² and R³ each can be C₁₋₃ alkyl, or R² and R³ can be taken together to form dioxolane;
R⁴ can be C₁₋₃ alkyl; and
R⁵ can be —C(O)—C₁₋₃ alkyl or —C(O)-phenyl.

In some embodiments, Compound 4, or a salt thereof, can yield Compound 5, or a salt thereof, in Step (i). In some embodiments, Step (i) can comprise (a) a disulfide cleavage and (b) a cyclization. In some embodiments, Compound 4, or a salt thereof, can undergo disulfide cleavage and cyclization to yield Compound 5, or a salt thereof, in Step (i). In some embodiments, the cyclization can be an acid catalyzed cyclization. In some embodiments, Compound 5, or a salt thereof, can yield Compound 6, or a salt thereof, in Step (ii). In some embodiments, Compound 5, or a salt thereof, can undergo a deprotection to yield Compound 6, or a salt thereof, in Step (ii). In some embodiments, a protecting group can be removed from Compound 5, or a salt thereof, to yield Compound 6, or a salt thereof, in Step (ii). In some embodiments, Compound 6, or a salt thereof, and Compound 7, or a salt thereof, can together yield compound 8, or a salt thereof, in Step (iii). In some embodiments, Compound 6, or a salt thereof, and Compound 7, or a salt thereof, can together undergo a coupling reaction to yield Compound 8, or a salt thereof, in Step (iii). In some embodiments, Compound 6, or a salt thereof, and Compound 7, or a salt thereof, can together undergo an amide coupling reaction to yield Compound 8, or a salt thereof, in Step (iii). In some embodiments, Compound 8, or a salt thereof, can yield Compound 1, or a salt thereof, in Step (iv). In some embodiments, Compound 8, or a salt thereof, can undergo a deprotection to yield Compound 1, or a salt thereof, in Step (iv). In some embodiments, a protecting group can be removed from Compound 8, or a salt thereof, to yield Compound 1, or a salt thereof, in Step (iv). In some embodiments, the acid and thiol protecting groups can be removed from Compound 8, or a salt thereof, to yield Compound 1, or a salt thereof, in Step (iv).

### Scheme A: Synthesis of Compound 4

In some embodiments, Compound 4 can be synthesized according to the following scheme.

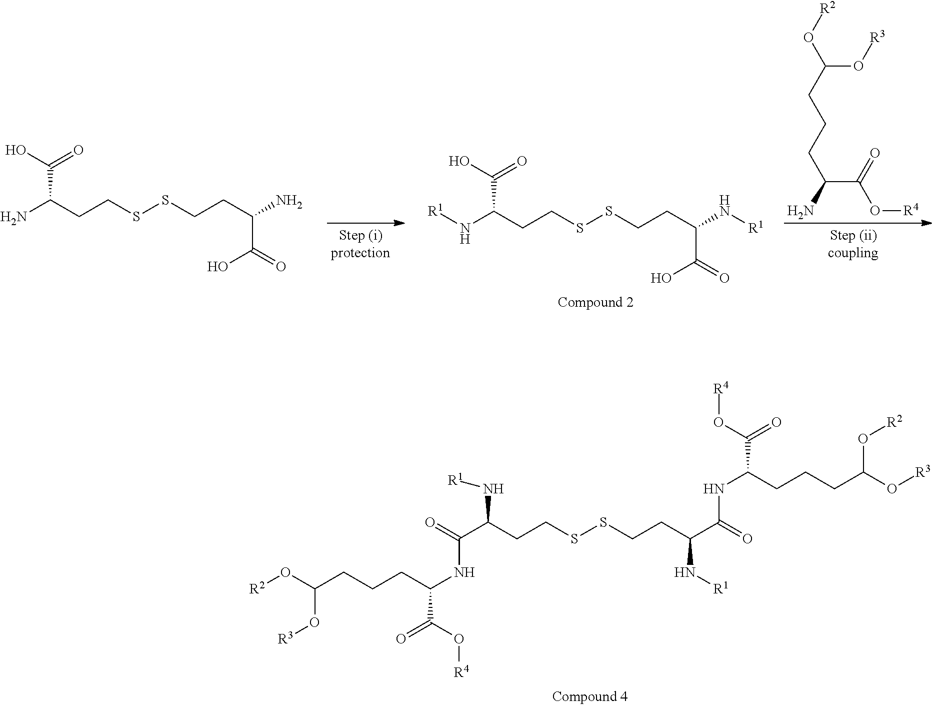

wherein R¹ can be a protecting group;
R² and R³ are each C₁₋₃ alkyl, or R² and R³ can be taken together to form dioxolane; and
R⁴ can be C₁₋₃ alkyl.

In some embodiments, (2S,2'S)-4,4′-disulfanediylbis(2-aminobutanoic acid), or a salt thereof, can yield Compound 2, or a salt thereof in Step (i). In some embodiments, Compound 2, or a salt thereof, can be prepared by mixing a suitable protecting group reagent with (2S,2'S)-4,4′-disulfanediylbis(2-aminobutanoic acid), or a salt thereof in Step (i). In some embodiments, each R¹ can be a protecting group. In some embodiments, each R¹ can be a nitrogen protecting group. In some embodiments, each R¹ can be an amine protecting group. In some embodiments, each R¹ can be a protecting group chosen from formyl, acetyl, trifluoroacetyl, benzyl carbamate, Fmoc, Boc, phthalimide, benzyl, trityl, benzylideneamine, and tosyl. In some embodiments, each R¹ can be a protecting group chosen from formyl, trifluoroacetyl, and benzyl carbamate. In some embodiments, each R¹ can be benzyl carbamate. In some embodiments, Compound 2, or a salt thereof, and Compound 3, or a salt thereof, can together yield Compound 4, or a salt thereof in Step (ii). In some embodiments, Compound 2, or a salt thereof, and Compound 3, or a salt thereof, can together undergo a coupling reaction to yield Compound 4, or a salt thereof, in Step (ii). In some embodiments, Compound 2, or a salt thereof, and Compound 3, or a salt thereof, can together undergo an amide coupling reaction to yield Compound 4, or a salt thereof, in Step (ii). In some embodiments, R² and R³ can each be C₁₋₃ alkyl. In some embodiments, R² and R³ can be taken together to form dioxolane. In some embodiments, R⁴ can be methyl.

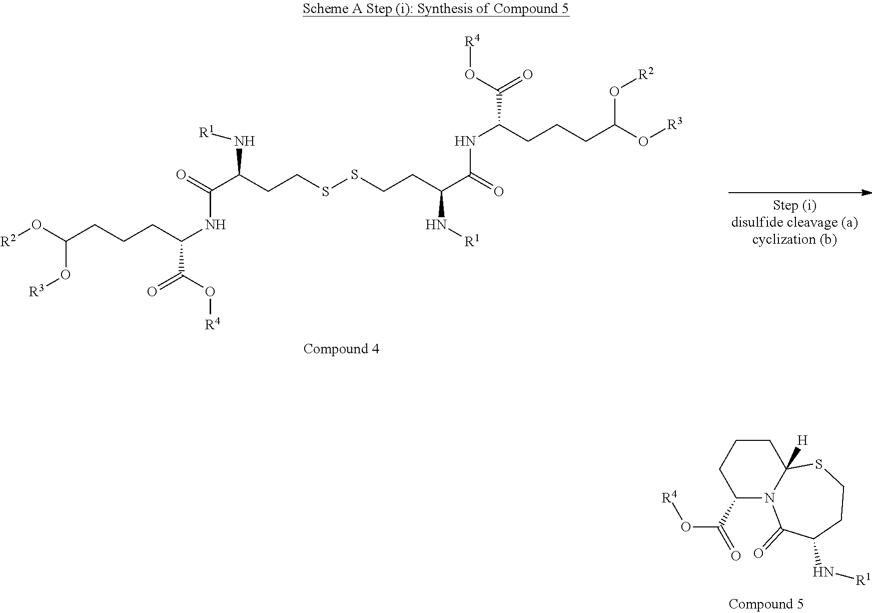

In some embodiments, Compound 5, or a salt thereof, can be prepared from Compound 4, or a salt thereof. In some embodiments, Compound 5 can be produced by a disulfide cleavage and cyclization of Compound 4. In some embodiments, Step (i) can comprise (a) a disulfide cleavage and (b) a cyclization.

Step (i)(a): Disulfide cleavage. In some embodiments, Compound 4, or a salt thereof, can be reacted with a reagent that cleaves disulfide bonds. In some embodiments, Compound 4, or a salt thereof, can be mixed with a reagent that cleaves disulfide bonds. In some embodiments, Compound 4, or a salt thereof, can be mixed in a suitable solvent with a reagent that cleaves disulfide bonds. In some embodiments, the suitable solvent can be a polar solvent. In some embodiments, the suitable solvent can be an aprotic solvent. In some embodiments, the solvent can be a non-polar solvent. In some embodiments, the solvent can be a protic solvent. In some embodiments, the solvent can be a mixture of solvents comprising one or more solvents. In some embodiments, the solvent can be methyl acetate or ethyl acetate. In some embodiments, the solvent can be methyl acetate. In some embodiments, the reagent that cleaves the disulfide bond can comprise a thiol or a phosphine. In some embodiments, the reagent that cleaves the disulfide bond can comprise a phosphine. In some embodiments, the reagent that cleaves the disulfide bond can be a phosphine selected from tris-(hydroxymethyl)phosphine, tris-(2-carboxyethyl)phosphine, tris(3-hydroxypropyl)phosphine, and tributyl phosphine, or combinations thereof. In some embodiments, the reagent that cleaves the disulfide bond can comprise a thiol. In some embodiments, the reagent that cleaves the disulfide bond can be a thiol selected from beta-mercaptoethanol, dithiothreitol, dithioerythritol, glutathione, N,N′- dimethyl-N,N′-bis(mercaptoacetyl)hydrazine, meso-2,5- dimercapto-N,N,N′,N′-tetramethyladipamide, bis(2-mercaptoethyl) sulfone, (2S)-2-amino-1,4-dimercaptobutane, 2,3-bis(mercaptomethyl)pyrazine, and 2-(dibenzylamino)butane-1,4-dithiol, or combinations thereof. In some embodiments, the reagent that cleaves the disulfide bond can be dithiothreitol. In some embodiments, the suitable solvent comprising Compound 4 can be cooled to a reduced temperature prior to the addition of the reagent that cleaves the disulfide bond. In some embodiments, the suitable solvent comprising Compound 4 can be cooled to about −40° C. to about 20° C. In some embodiments, the solvent comprising Compound 4 can be cooled to about −35° C. to about 15° C. In some embodiments, the solvent comprising Compound 4 can be cooled to about −30° C. to about 10° C. In some embodiments, the solvent comprising Compound 4 can be cooled to about −25° C. to about 5° C. In some embodiments, the solvent comprising Compound 4 can be cooled to about −20° C. to about 0° C. In some embodiments, the solvent comprising Compound 4 can be cooled to about −15° C. to about −5° C. In some embodiments, the solvent comprising Compound 4 can be cooled to about −10° C. In some embodiments, the solvent comprising Compound 4 can be cooled to −10° C. ±5° C. In some embodiments, the solvent comprising Compound 4 can be cooled to 10° C. ±3° C. In some embodiments, Step (i)(a) can comprise about 200 mL to about 50,000 mL of solvent. In some embodiments, Step (i)(a) can comprise about 500 mL to about 50,000 mL of solvent. In some embodiments, Step (i)(a) can comprise about 1,000 mL to about 50,000 mL of solvent. In some embodiments, Step (i)(a) can comprise about 2,000 mL to about 50,000 mL of solvent. In some embodiments, Step (i)(a) can comprise about 4,000 mL to about 50,000 mL of solvent. In some embodiments, Step (i)(a) can comprise about 8,000 mL to about 50,000 mL of solvent. In some embodiments, Step (i)(a) can comprise about 16,000 mL to about 50,000 mL of solvent. In some embodiments, Step (i)(a) can comprise about 32,000 mL to about 50,000 mL of solvent. In some embodiments, Step (i)(a) can comprise about 200 mL to about 40,000 mL of solvent. In some embodiments, Step (i)(a) can comprise about 200 mL to about 20,000 mL of solvent. In some embodiments, Step (i)(a) can comprise about 200 mL to about 15,000 mL of solvent. In some embodiments, Step (i)(a) can comprise about 200 mL to about 8,000 mL of solvent. In some embodiments, Step (i)(a) can comprise about 200 mL to about 4,000 mL of solvent. In some embodiments, Step (i)(a) can comprise about 200 mL to about 2,000 mL of solvent. In some embodiments, Step (i)(a) can comprise about 200 mL to about 1,000 mL of solvent. In some embodiments, Step (i)(a) can comprise about 200 mL to about 500 mL of solvent. In some embodiments, Step (i)(a) can comprise about 500 mL to about 1,200 mL of solvent. In some embodiments, Step (i)(a) can comprise about 500 mL to about 1,500 mL of solvent. In some embodiments, Step (i)(a) can comprise about 1,000 mL to about 3,000 mL of solvent. In some embodiments, Step (i)(a) can comprise about 2,000 mL to about 4,000 mL of solvent. In some embodiments, Step (i)(a) can comprise about 4,000 mL to about 6,000 mL of solvent. In some embodiments, Step (i)(a) can comprise about 5,000 mL to about 7,000 mL of solvent. In some embodiments, Step (i)(a) can comprise about 6,000 mL to about 8,000 mL of solvent. In some embodiments, Step (i)(a) can comprise about 7,000 mL to about 9,000 mL of solvent. In some embodiments, Step (i)(a) can comprise about 8,000 mL to about 10,000 mL of solvent. In some embodiments, Step (i)(a) can comprise about 9,000 mL to about 11,000 mL of solvent. In some embodiments, Step (i)(a) can comprise about 10,000 mL to about 12,000 mL of solvent. In some embodiments, Step (i)(a) can comprise about 11,000 mL to about 13,000 mL of solvent. In some embodiments, Step (i)(a) can comprise about 12,000 mL to about 14,000 mL of solvent. In some embodiments, Step (i)(a) can comprise about 13,000 mL to about 15,000 mL of solvent. In some embodiments, Step (i)(a) can comprise about 14,000 mL to about 16,000 mL of solvent. In some embodiments, Step (i)(a) can comprise about 15,000 mL to about 17,000 mL of solvent. In some embodiments, Compound 4, or a salt thereof, can be mixed in a suitable solvent with a reagent that cleaves disulfide bonds and a base. In some embodiments, the base can be an organic base. In some embodiments, the base can be an inorganic base. In some embodiments, the base can be a reducing agent. In some embodiments, the base can be sodium methoxide.

In some embodiments, each R¹ can be a protecting group. In some embodiments, each R¹ can be a nitrogen protecting group. In some embodiments, each R¹ can be an amine protecting group. In some embodiments, each R¹ can be a protecting group chosen from formyl, acetyl, trifluoroacetyl, benzyl carbamate, Fmoc, Boc, phthalimide, benzyl, trityl, benzylideneamine, and tosyl. In some embodiments, each R¹ can be a protecting group chosen from formyl, trifluoroacetyl, and benzyl carbamate. In some embodiments, each R¹ can be benzyl carbamate.

In some embodiments, each R² and R³ can be each C₁₋₃ alkyl. In some embodiments, R² and R³ each can be C₁₋₂ alkyl. In some embodiments, each R² and R³ can be propyl. In some embodiments, each R² and R³ can be ethyl. In some embodiments, each R² and R³ can be methyl. In some embodiments, R² and R³ can be taken together to form dioxolane.

In some embodiments, each R⁴ can be each C₁₋₃ alkyl. In some embodiments, each R⁴ can be C₁₋₂alkyl. In some embodiments, each R⁴ can be propyl. In some embodiments, each R⁴ can be each ethyl. In some embodiments, each R⁴ can be methyl.

In some embodiments, Compound 4 can be Compound 4a or Compound 4b, or a salt thereof:

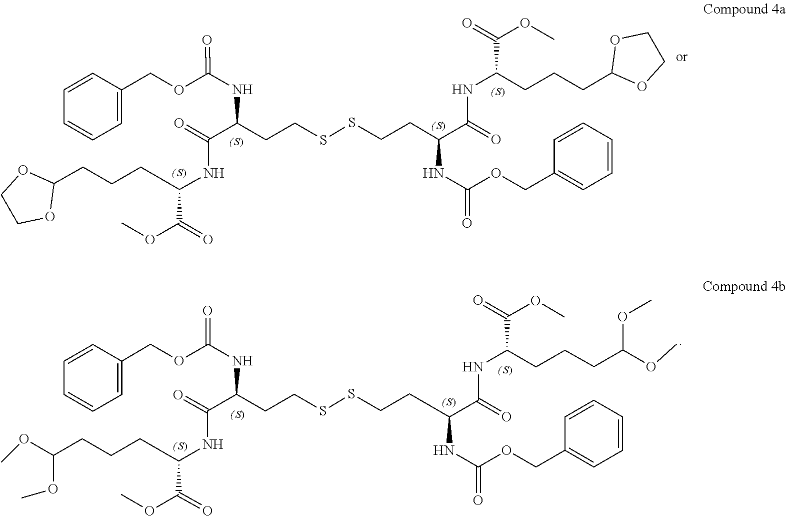

In some embodiments, Compound 4 can be Compound 4a or a salt thereof:

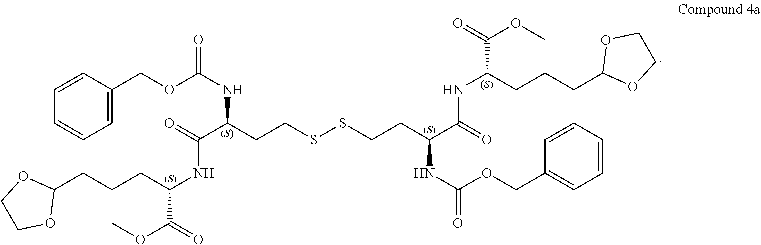

In some embodiments, Compound 4 can be converted to a monomer in Step(i)(a):

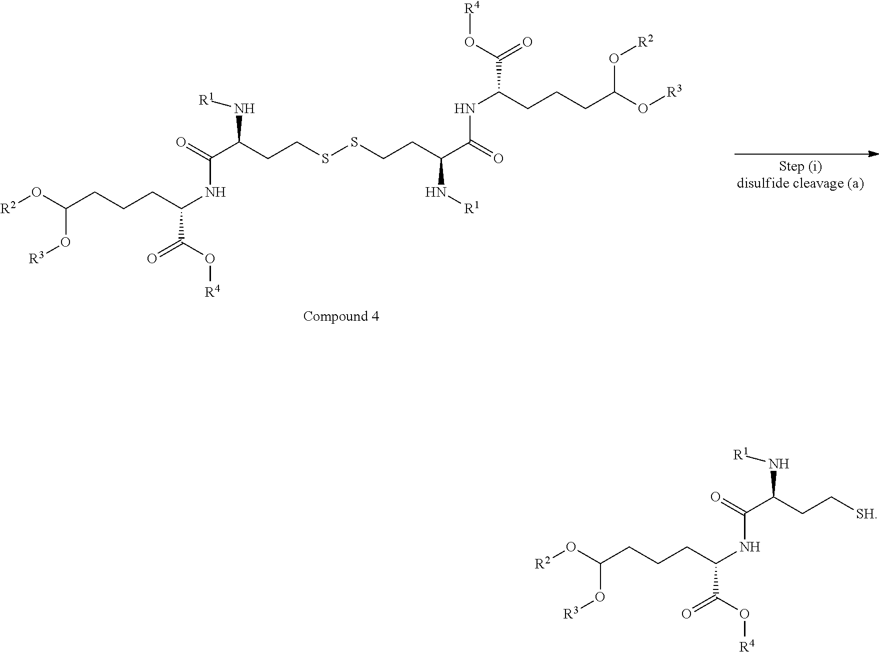

In some embodiments, Compound 4 can be converted to a monomer in Step(i)(a) having a structure of

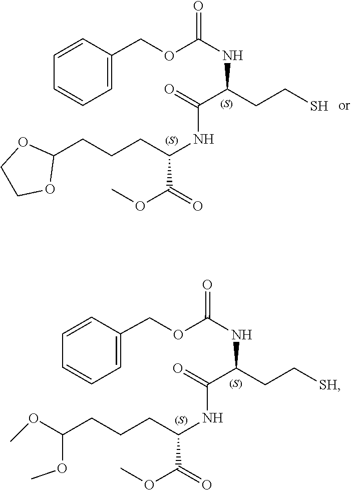

or a salt thereof. In some embodiments, Compound 4 can be converted to a monomer in Step(i)(a) having a structure of

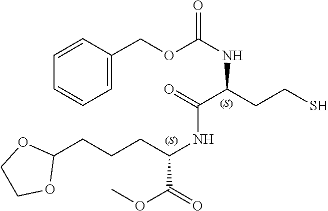

or a salt thereof. In some embodiments, the monomer intermediate from Step (i)(a) can be isolated by precipitation and filtration. In some embodiments, the monomer intermediate from Step (i)(a) can be isolated by extraction. In some embodiments, the monomer intermediate from Step (i)(a) can be purified before Step (i)(b). In some embodiments, the monomer intermediate from Step (i)(a) is not purified before Step (i)(b). In some embodiments, the monomer intermediate from Step (i)(a) can undergo a cyclization reaction to form Compound 5 in Step (i)(b).

Step (i)(b): Cyclization. In some embodiments, the monomer from Step (i)(a) can yield Compound 5 in Step (i)(b). In some embodiments, the monomer from Step (i)(a) can undergo a cyclization reaction in a suitable solvent to yield Compound 5, or a salt thereof. In some embodiments, the monomer from Step (i)(a) can undergo an acid catalyzed cyclization reaction in a suitable solvent to yield Compound 5, or a salt thereof, in Step (i)(b). In some embodiments, the suitable solvent can be polar. In some embodiments, the suitable solvent can be aprotic. In some embodiments, the solvent can be a mixture of solvents comprising one or more solvents. In some embodiments, the suitable solvent can comprise acetonitrile. In some embodiments, the suitable solvent can comprise water. In some embodiments, the suitable solvent can comprise acetonitrile and water. In some embodiments, the suitable solvent can comprise a suitable solvent and water. In some embodiments, the ratio between the volumes of suitable solvent and water can be between 1,000:1 and 1:1. In some embodiments, the ratio between the volumes of suitable solvent and water can be between 80:1 and 1:1. In some embodiments, the ratio between the volumes of suitable solvent and water can be between 60:1 and 1:1. In some embodiments, the ratio between the volumes of suitable solvent and water can be between 40:1 and 1:1. In some embodiments, the ratio between the volumes of suitable solvent and water can be between 500:1 and 40:1. In some embodiments, the ratio between the volumes of suitable solvent and water can be between 200:1 and 40:1. In some embodiments, the ratio between the volumes of suitable solvent and water can be between 100:1 and 40:1. In some embodiments, the ratio between the volumes of suitable solvent and water can be between 90:1 and 50:1. In some embodiments, the ratio between the volumes of suitable solvent and water can be between 80:1 and 60:1. In some embodiments, the suitable solvent comprises less than about 50% water by volume. In some embodiments, the suitable solvent comprises less than about 40% water by volume. In some embodiments, the suitable solvent comprises less than about 30% water by volume. In some embodiments, the suitable solvent comprises less than about 20% water by volume. In some embodiments, the suitable solvent comprises less than about 10% water by volume. In some embodiments, the suitable solvent comprises less than about 5% water by volume. In some embodiments, the suitable solvent comprises less than about 3% water by volume. In some embodiments, the suitable solvent comprises less than about 1% water by volume. In some embodiments, Step (i)(b) can comprise about 200 mL to about 50,000 mL of solvent. In some embodiments, Step (i)(b) can comprise about 500 mL to about 50,000 mL of solvent. In some embodiments, Step (i)(b) can comprise about 1,000 mL to about 50,000 mL of solvent. In some embodiments, Step (i)(b) can comprise about 2,000 mL to about 50,000 mL of solvent. In some embodiments, Step (i)(b) can comprise about 4,000 mL to about 50,000 mL of solvent. In some embodiments, Step (i)(b) can comprise about 8,000 mL to about 50,000 mL of solvent. In some embodiments, Step (i)(b) can comprise about 16,000 mL to about 50,000 mL of solvent. In some embodiments, Step (i)(b) can comprise about 32,000 mL to about 50,000 mL of solvent. In some embodiments, Step (i)(b) can comprise about 200 mL to about 40,000 mL of solvent. In some embodiments, Step (i)(b) can comprise about 200 mL to about 20,000 mL of solvent. In some embodiments, Step (i)(b) can comprise about 200 mL to about 15,000 mL of solvent. In some embodiments, Step (i)(b) can comprise about 200 mL to about 8,000 mL of solvent. In some embodiments, Step (i)(b) can comprise about 200 mL to about 4,000 mL of solvent. In some embodiments, Step (i)(b) can comprise about 200 mL to about 2,000 mL of solvent. In some embodiments, Step (i)(b) can comprise about 200 mL to about 1,000 mL of solvent. In some embodiments, Step (i)(b) can comprise about 200 mL to about 500 mL of solvent. In some embodiments, Step (i)(b) can comprise about 200 mL to about 1,200 mL of solvent. In some embodiments, Step (i)(b) can comprise about 200 mL to about 1,500 mL of solvent. In some embodiments, Step (i)(b) can comprise about 1,000 mL to about 3,000 mL of solvent. In some embodiments, Step (i)(b) can comprise about 2,000 mL to about 4,000 mL of solvent. In some embodiments, Step (i)(b) can comprise about 4,000 mL to about 6,000 mL of solvent. In some embodiments, Step (i)(b) can comprise about 5,000 mL to about 7,000 mL of solvent. In some embodiments, Step (i)(b) can comprise about 6,000 mL to about 8,000 mL of solvent. In some embodiments, Step (i)(b) can comprise about 7,000 mL to about 9,000 mL of solvent. In some embodiments, Step (i)(b) can comprise about 8,000 mL to about 10,000 mL of solvent. In some embodiments, Step (i)(b) can comprise about 9,000 mL to about 11,000 mL of solvent. In some embodiments, Step (i)(b) can comprise about 10,000 mL to about 12,000 mL of solvent. In some embodiments, Step (i)(b) can comprise about 11,000 mL to about 13,000 mL of solvent. In some embodiments, Step (i)(b) can comprise about 12,000 mL to about 14,000 mL of solvent. In some embodiments, Step (i)(b) can comprise about 13,000 mL to about 15,000 mL of solvent. In some embodiments, Step (i)(b) can comprise about 14,000 mL to about 16,000 mL of solvent. In some embodiments, Step (i)(b) can comprise about 15,000 mL to about 17,000 mL of solvent. In some embodiments, the acid catalyzed cyclization can comprise mixing the monomer from Step (i)(a) in a suitable solvent with an acid. In some embodiments, the acid can be trifluoroacetic acid, chlorosulfonic acid, p-toluenesulfonic acid, methanesulfonic acid, trifluoromethanesulfonic acid, trimethylsilyl trifluoromethanesulfonate, trimethylsilyl methanesulfonate, Amberlyst, or a combination thereof. In some embodiments, the acid can be trifluoroacetic acid. In some embodiments, Compound 5 can be isolated by precipitation and filtration. In some embodiments, Compound 5 can be isolated by extraction. In some embodiments, Compound 5 can be purified via column chromatography. In some embodiments, Compound 5 can be purified via precipitation. In some embodiments, Compound 5 can be purified via crystallization. In some embodiments, Compound 5 can be purified via recrystallization.

In some embodiments, R⁴ can be C₁₋₃ alkyl, R¹ can be benzyl carbamate, and Compound 5 can have the structure of

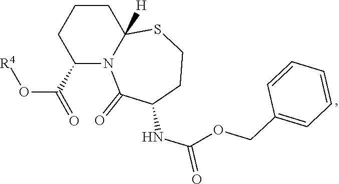

or a salt thereof. In some embodiments, R⁴ can be methyl, R¹ can be benzyl carbamate, and Compound 5 can have the structure of

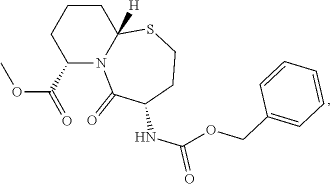

or a salt thereof.

In some embodiments, the protecting group R¹ can impact yield of Compound 5. In some embodiments, R¹ can be benzyl carbamate, and Compound 5 can be isolated with higher yield. In some embodiments, R¹ can be benzyl carbamate, and Compound 5 can be isolated with higher yield compared to when R¹ is not benzyl carbamate. In some embodiments, R¹ can be benzyl carbamate, and Compound 5 can be isolated with a higher yield compared to formyl or trifluoroacetate protecting groups.

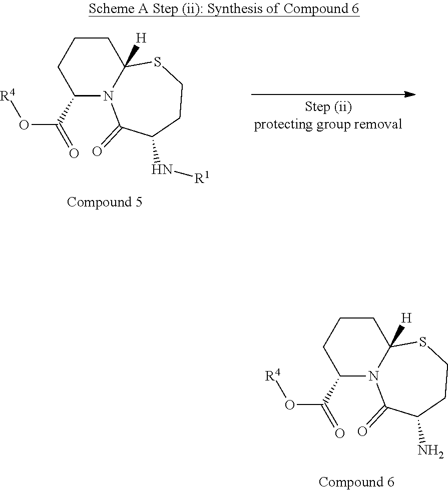

In some embodiments, Compound 6, or a salt thereof, can be prepared from Compound 5, or a salt thereof. In some embodiments, Compound 6, or a salt thereof, can be produced by a deprotection reaction of Compound 5, or a salt thereof. In some embodiments, Compound 5, or a salt thereof can undergo a deprotection reaction to yield Compound 6, or a salt thereof. In some embodiments, Compound 5 can be mixed with a suitable reagent to yield Compound 6. In some embodiments, Compound 5 can be mixed with a suitable reagent in a suitable solvent to yield Compound 6. In some embodiments, the suitable reagent can comprise an acid, a base, palladium, or an organosilicon compound. In some embodiments, the suitable reagent can comprise an acid, a base, or palladium. In some embodiments, the suitable solvent can comprise a polar solvent, a nonpolar solvent, an aprotic solvent, or a protic solvent. In some embodiments, the suitable solvent can comprise a polar solvent. In some embodiments, the suitable solvent can comprise an aprotic solvent. In some embodiments, the suitable solvent can be a mixture of solvents comprising one or more solvents. In some embodiments, the suitable solvent can be dichloromethane. In some embodiments, Step (ii) can comprise about 200 mL to about 50,000 mL of solvent. In some embodiments, Step (ii) can comprise about 500 mL to about 50,000 mL of solvent. In some embodiments, Step (ii) can comprise about 1,000 mL to about 50,000 mL of solvent. In some embodiments, Step (ii) can comprise about 2,000 mL to about 50,000 mL of solvent. In some embodiments, Step (ii) can comprise about 4,000 mL to about 50,000 mL of solvent. In some embodiments, Step (ii) can comprise about 8,000 mL to about 50,000 mL of solvent. In some embodiments, Step (ii) can comprise about 16,000 mL to about 50,000 mL of solvent. In some embodiments, Step (ii) can comprise about 32,000 mL to about 50,000 mL of solvent. In some embodiments, Step (ii) can comprise about 200 mL to about 40,000 mL of solvent. In some embodiments, Step (ii) can comprise about 200 mL to about 20,000 mL of solvent. In some embodiments, Step (ii) can comprise about 200 mL to about 15,000 mL of solvent. In some embodiments, Step (ii) can comprise about 200 mL to about 8,000 mL of solvent. In some embodiments, Step (ii) can comprise about 200 mL to about 4,000 mL of solvent. In some embodiments, Step (ii) can comprise about 200 mL to about 2,000 mL of solvent. In some embodiments, Step (ii) can comprise about 200 mL to about 1,000 mL of solvent. In some embodiments, Step (ii) can comprise about 200 mL to about 500 mL of solvent. In some embodiments, Step (ii) can comprise about 500 mL to about 1,200 mL of solvent. In some embodiments, Step (ii) can comprise about 500 mL to about 1,500 mL of solvent. In some embodiments, Step (ii) can comprise about 1,000 mL to about 3,000 mL of solvent. In some embodiments, Step (ii) can comprise about 2,000 mL to about 4,000 mL of solvent. In some embodiments, Step (ii) can comprise about 4,000 mL to about 6,000 mL of solvent. In some embodiments, Step (ii) can comprise about 5,000 mL to about 7,000 mL of solvent. In some embodiments, Step (ii) can comprise about 6,000 mL to about 8,000 mL of solvent. In some embodiments, Step (ii) can comprise about 7,000 mL to about 9,000 mL of solvent. In some embodiments, Step (ii) can comprise about 8,000 mL to about 10,000 mL of solvent. In some embodiments, Step (ii) can comprise about 9,000 mL to about 11,000 mL of solvent. In some embodiments, Step (ii) can comprise about 10,000 mL to about 12,000 mL of solvent. In some embodiments, Step (ii) can comprise about 11,000 mL to about 13,000 mL of solvent. In some embodiments, Step (ii) can comprise about 12,000 mL to about 14,000 mL of solvent. In some embodiments, Step (ii) can comprise about 13,000 mL to about 15,000 mL of solvent. In some embodiments, Step (ii) can comprise about 14,000 mL to about 16,000 mL of solvent. In some embodiments, Step (ii) can comprise about 15,000 mL to about 17,000 mL of solvent. In some embodiments, R¹ can be Boc and the suitable reagent can comprise an acid. In some embodiments, R¹ can be Boc and the suitable reagent can comprise an acid, wherein the acid can be sulfuric acid, formic acid, HCl, or trifluoroacetic acid. In some embodiments, R¹ can be Boc and the suitable reagent can comprise an acid, wherein the acid can be HCl or trifluoroacetic acid. In some embodiments, R¹ can be formyl and the suitable reagent can comprise an acid or a base. In some embodiments, R¹ can be formyl and the suitable reagent can comprise a base. In some embodiments, R¹ can be formyl and the suitable reagent can comprise an acid, wherein the acid can be HCl or trifluoroacetic acid. In some embodiments, R¹ can be acetyl and the suitable reagent can comprise an acid or a base. In some embodiments, R¹ can be acetyl and the suitable reagent can comprise an acid. In some embodiments, R¹ can be acetyl and the suitable reagent can comprise a base, wherein the base can be pyridine, NaOH, or KOH. In some embodiments, R¹ can be trifluoroacetyl and the suitable reagent can comprise a base. In some embodiments, R¹ can be trifluoroacetyl and the suitable reagent can comprise a base, wherein the base can comprise carbonate or alkoxide. In some embodiments, R¹ can be benzyl carbamate and the suitable reagent can comprise hydrogen. In some embodiments, R¹ can be benzyl carbamate and the suitable reagent can comprise hydrogen and a catalyst. In some embodiments, R¹ can be benzyl carbamate and the suitable reagent can comprise hydrogen and the catalyst can comprise palladium. In some embodiments, R¹ can be benzyl carbamate and the suitable reagent can comprise an acid. In some embodiments, R¹ can be benzyl carbamate and the suitable reagent can comprise an acid, wherein the acid can have a pH value of about <3. In some embodiments, R¹ can be benzyl carbamate and the suitable reagent can comprise an acid, wherein the acid can have a pH value of about <2. In some embodiments, R¹ can be benzyl carbamate and the suitable reagent can comprise an acid, wherein the acid can have a pH value of about <1. In some embodiments, R¹ can be benzyl carbamate and the suitable reagent can comprise an acid, wherein the acid can be a Lewis acid (e.g., TMS-I), HCl, or trifluoroacetic acid. In some embodiments, R¹ can be Fmoc and the suitable reagent can comprise a base. In some embodiments, R¹ can be Fmoc and the suitable reagent can comprise an amine. In some embodiments, the amine can be cyclohexylamine, ethanolamine, piperidine, piperazine, diisopropylethylamine, or triethylamine. In some embodiments, R¹ can be phthalimide and the suitable reagent can be hydrazine. In some embodiments, R¹ can be benzyl and the suitable reagent can comprise palladium and hydrogen. In some embodiments, R¹ can be trityl and the suitable reagent can comprise an acid. In some embodiments, R¹ can be trityl and the suitable reagent can comprise an acid, wherein the acid can be HCl or trifluoroacetic acid. In some embodiments, R¹ can be benzylideneamine. In some embodiments, R¹ can be tosyl and the suitable reagent can comprise an acid or a reducing agent. In some embodiments, R¹ can be tosyl and the suitable reagent can comprise an acid. In some embodiments, R¹ can be tosyl and the suitable reagent can comprise an acid, wherein the acid can comprise HBr or acetic acid. In some embodiments, R¹ can be tosyl and the suitable reagent can comprise a reducing agent. In some embodiments, R¹ can be tosyl and the suitable reagent can comprise a reducing agent, wherein the reducing agent can comprise SmI2 or SMEAH. In some embodiments, R⁴ can be methyl and Compound 6 can have the structure of

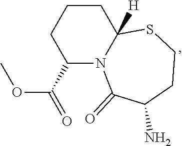

or a salt thereof. In some embodiments, Compound 6 can be isolated by precipitation and filtration. In some embodiments, Compound 6 can be isolated by extraction. In some embodiments, Compound 6 can be purified via column chromatography. In some embodiments, Compound 6 can be purified via precipitation. In some embodiments, Compound 6 can be purified via crystallization. In some embodiments, Compound 6 can be purified via recrystallization. In some embodiments, Compound 6 is purified via precipitation as a salt. In some embodiments, the salt is an HCl or an HI salt. In some embodiments, the salt is an HCl salt. In some embodiments, the salt is an HI salt.

In some embodiments, the protecting group R¹ can impact yield of Compound 6. In some embodiments, R¹ can be benzyl carbamate, and Compound 6 can be isolated with higher yield. In some embodiments, R¹ can be benzyl carbamate, and Compound 6 can be isolated with higher yield compared to when R¹ is not benzyl carbamate. In some embodiments, R¹ can be benzyl carbamate, and Compound 6 can be isolated with a higher yield compared to formyl or trifluoroacetate protecting groups.

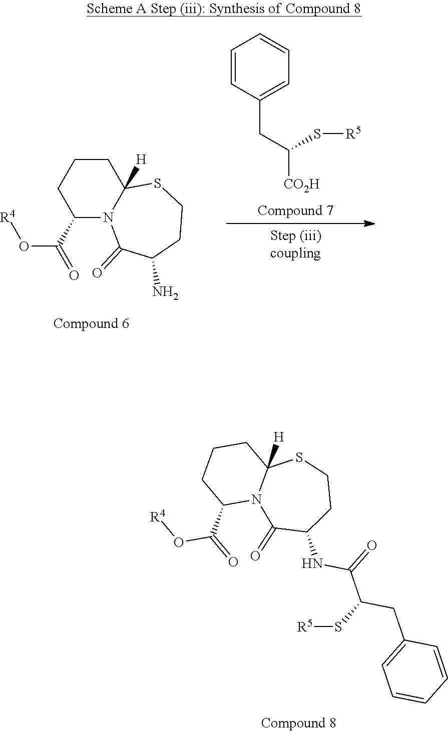

In some embodiments, Compound 8, or a salt thereof, can be prepared from Compound 6, or a salt thereof, and Compound 7, or a salt thereof. In some embodiments, Compound 8, or a salt thereof, can be produced by a coupling reaction of Compound 6, or a salt thereof, and Compound 7, or a salt thereof. In some embodiments, Compound 6, or a salt thereof can undergo a coupling reaction with Compound 7, or a salt thereof to yield Compound 8, or a salt thereof. In some embodiments, Compound 6 can be mixed with Compound 7 in the presence of a coupling reagent and in a suitable solvent to yield Compound 8. In some embodiments, Step (iii) can comprise about 200 mL to about 50,000 mL of solvent. In some embodiments, Step (iii) can comprise about 500 mL to about 50,000 mL of solvent. In some embodiments, Step (iii) can comprise about 1,000 mL to about 50,000 mL of solvent. In some embodiments, Step (iii) can comprise about 2,000 mL to about 50,000 mL of solvent. In some embodiments, Step (iii) can comprise about 4,000 mL to about 50,000 mL of solvent. In some embodiments, Step i(ii) can comprise about 8,000 mL to about 50,000 mL of solvent. In some embodiments, Step (iii) can comprise about 16,000 mL to about 50,000 mL of solvent. In some embodiments, Step (iii) can comprise about 32,000 mL to about 50,000 mL of solvent. In some embodiments, Step (iii) can comprise about 200 mL to about 40,000 mL of solvent. In some embodiments, Step (iii) can comprise about 200 mL to about 20,000 mL of solvent. In some embodiments, Step (iii) can comprise about 200 mL to about 15,000 mL of solvent. In some embodiments, Step (iii) can comprise about 200 mL to about 8,000 mL of solvent. In some embodiments, Step (iii) can comprise about 200 mL to about 4,000 mL of solvent. In some embodiments, Step (iii) can comprise about 200 mL to about 2,000 mL of solvent. In some embodiments, Step (iii) can comprise about 200 mL to about 1,000 mL of solvent. In some embodiments, Step (iii) can comprise about 200 mL to about 500 mL of solvent. In some embodiments, Step (iii) can comprise about 500 mL to about 1,200 mL of solvent. In some embodiments, Step (iii) can comprise about 500 mL to about 1,500 mL of solvent. In some embodiments, Step (iii) can comprise about 1,000 mL to about 3,000 mL of solvent. In some embodiments, Step (iii) can comprise about 2,000 mL to about 4,000 mL of solvent. In some embodiments, Step (iii) can comprise about 4,000 mL to about 6,000 mL of solvent. In some embodiments, Step (iii) can comprise about 5,000 mL to about 7,000 mL of solvent. In some embodiments, Step (iii) can comprise about 6,000 mL to about 8,000 mL of solvent. In some embodiments, Step (iii) can comprise about 7,000 mL to about 9,000 mL of solvent. In some embodiments, Step (iii) can comprise about 8,000 mL to about 10,000 mL of solvent. In some embodiments, Step (iii) can comprise about 9,000 mL to about 11,000 mL of solvent. In some embodiments, Step (iii) can comprise about 10,000 mL to about 12,000 mL of solvent. In some embodiments, Step (iii) can comprise about 11,000 mL to about 13,000 mL of solvent. In some embodiments, Step (iii) can comprise about 12,000 mL to about 14,000 mL of solvent. In some embodiments, Step (iii) can comprise about 13,000 mL to about 15,000 mL of solvent. In some embodiments, Step (iii) can comprise about 14,000 mL to about 16,000 mL of solvent. In some embodiments, Step (iii) can comprise about 15,000 mL to about 17,000 mL of solvent. In some embodiments, a coupling reagent can comprise a reagent that facilitates a formation of an amide bond. In some embodiments, a coupling reagent can comprise a pivaloyl chloride, an isobutyl chloroformate, a benzotriazol-1-yloxytripyrrolidinophosphonium hexafluorophosphate (PyBOP), a propylphosphonic anhydride (T3P), (benzotriazol-1-yloxy)tris(dimethylamino)phosphonium hexafluorophosphate (BOP), an ethyl-(N′,N′-dimethylamino)propylcarbodiimide (EDC), a (1-Cyano-2-ethoxy-2-oxoethylidenaminooxy)dimethylamino-morpholino-carbenium hexafluorophosphate (COMU), a 3-(diethylphosphoryloxy)-1,2,3-benzotriazin-4(3H)-one (DEPBT), an O-(7-azabenzotriazol-1-yl)-N,N,N′,N′-tetramethyluronium hexafluorophosphate (HATU), an O-(benzotriazol-1-yl)-N,N,N′,N′-tetramethyluronium hexafluorophosphate (HBTU), an O-(6-chlorobenzotriazol-1-yl)-N,N,N′,N′-tetramethyluronium hexafluorophosphate (HCTU), an O-(7-azabenzotriazol-1-yl)- N,N,N′,N′-tetramethyluronium tetrafluoroborate (TATU), an O-(benzotriazol-1-yl)- N,N,N′,N′-tetramethyluronium tetrafluoroborate (TBTU), a (7-azabenzotriazol-1-yloxy)tripyrrolidinophosphonium hexafluorophosphate (PyAOP), a bromotripyrrolidinophosphonium hexafluorophosphate (PyBrOP), a 6-chloro-benzotriazole-1-yloxy-tris-pyrrolidinophosphonium hexafluorophosphate (PyClock), a chlorotripyrrolidinophosphonium hexafluorophosphate (PyCloP), a bis(2-oxo-3-oxazolidinyl)phosphinic chloride (BOP—Cl), [ethyl cyano(hydroxyimino)acetato-O²]tri-1-pyrrolidinylphosphonium hexafluorophosphate (PyOxim), an O—(N-succinimidyl)-1,1,3,3-tetramethyl-uronium tetrafluoroborate (TSTU), an O-(5-norbornene-2,3-dicarboximido)-N,N,N′,N′-tetramethyluronium tetrafluoroborate (TNTU), an O-(1,2-dihydro-2-oxo-1-pyridyl-N,N,N′,N′-tetramethyluronium tetrafluoroborate (TPTU), a diisopropylcarbodiimide (DIC), a carbonyldiimidazole (CDI), a dicyclohexylcarbodiimide (DCC), an O-[(ethoxycarbonyl)cyanomethyleneamino]-N,N,N‘N’-tetramethyluronium tetrafluoroborate (TOTU), a N,N,N′,N′-tetramethyl-O-(3,4-dihydro-4-oxo-1,2,3-benzotriazin-3-yl)uronium tetrafluoroborate (TDBTU), a N,N,N′,N′-tetramethylchloroformamidinium hexafluorophosphate (TCFH), a salt of any of these, a stereoisomer of any of these, or any combination thereof. In some embodiments, the coupling reagent can comprise propylphosphonic anhydride (T3P) or (benzotriazol-1-yloxy)tripyrrolidinophosphonium hexafluorophosphate (PyBOP). In some embodiments, the coupling reagent can comprise propylphosphonic anhydride (T3P). In some embodiments, the coupling reagent can comprise benzotriazol-1-yloxytripyrrolidinophosphonium hexafluorophosphate (PyBOP). In some embodiments, the coupling reagent can comprise a reagent that can produce water soluble reaction by-products. In some embodiments, Compound 6 can be mixed with Compound 7 in the presence of the coupling reagent, and a base, and in a suitable solvent to yield Compound 8. In some embodiments, the base can be an organic base. In some embodiments, the organic base can be TEA, N,N-diethylaniline, DIPEA, DBU, pyridine, or N-methyl morpholine. In some embodiments, the organic base can be TEA. In some embodiments, the suitable solvent can be a polar solvent. In some embodiments, the suitable solvent can be an aprotic solvent. In some embodiments, the suitable solvent can be a polar aprotic solvent. In some embodiments, the suitable solvent can be dimethylformamide, dichloromethane, tetrahydrofuran, or methyl-tetrahydrofuran. In some embodiments, the solvent can be tetrahydrofuran or methyl-tetrahydrofuran. In some embodiments, the suitable solvent can be dimethylformamide or dichloromethane. In some embodiments, Compound 6, the organic base, and the coupling reagent can be added to Compound 7 dissolved in a suitable solvent, wherein the solvent can be cooled to a reduced temperature. In some embodiments, the reduced temperature can be between about 0° C. to about 15° C. In some embodiments, the reduced temperature can be between about 0° C. to about 5° C. In some embodiments, the reduced temperature can be between about −5° C. to about 5° C.

In some embodiments, R⁵ can be —C(O)—C₁₋₃ alkyl or —C(O)-phenyl. In some embodiments, R⁵ can be —C(O)—C₁₋₃ alkyl. In some embodiments, R⁵ can be —C(O)—C₁₋₂alkyl. In some embodiments, R⁵ can be —C(O)-propyl. In some embodiments, R⁵ can be —C(O)-ethyl. In some embodiments, R⁵ can be —C(O)-methyl. In some embodiments, Compound 7 can have the structure of

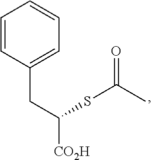

or a salt thereof. In some embodiments, R⁵ can be —C(O)-phenyl. In some embodiments, Compound 7 can have the structure of

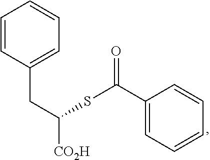 [SMILES: O=C(S[C@@H](Cc1ccccc1)C(=O)O)c1ccccc1]

or a salt thereof. In some embodiments, Compound 7 can be stereochemically enriched. In some embodiments, Compound 7 can be optically pure. In some embodiments, the optical purity of Compound 7 can be greater than about 70% enantiomeric excess. In some embodiments, the optical purity of Compound 7 can be greater than about 75% enantiomeric excess. In some embodiments, the optical purity of Compound 7 can be greater than about 80% enantiomeric excess. In some embodiments, the optical purity of Compound 7 can be greater than about 85% enantiomeric excess. In some embodiments, the optical purity of Compound 7 can be greater than about 90% enantiomeric excess. In some embodiments, the optical purity of Compound 7 can be greater than about 95% enantiomeric excess.

In some embodiments, Compound 7 can have the structure of

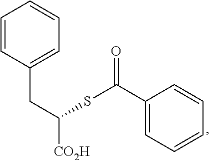 [SMILES: O=C(S[C@@H](Cc1ccccc1)C(=O)O)c1ccccc1]

or a salt thereof, and Compound 8 can have the structure of

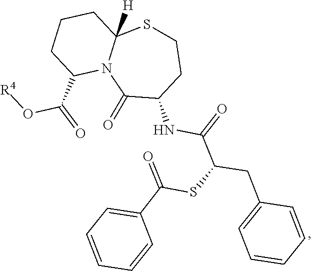

or a salt thereof. In some embodiments, R⁴ can be C₁₋₃ alkyl. In some embodiments, R⁴ can be methyl. In some embodiments, Compound 8 can have the structure of

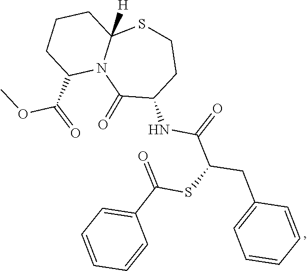

or a salt thereof. In some embodiments, Compound 7 can have the structure of

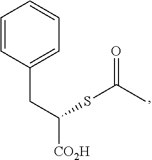

or a salt thereof, and Compound 8 can have the structure of

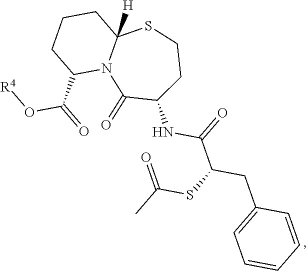

or a salt thereof. In some embodiments, R⁴ can be C₁₋₃ alkyl. In some embodiments, R⁴ can be methyl. In some embodiments, Compound 8 can have the structure of

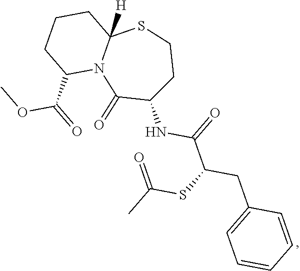

or a salt thereof. In some embodiments, Compound 8 can be isolated by precipitation and filtration. In some embodiments, Compound 8 can be isolated by extraction. In some embodiments, Compound 8 can be purified via column chromatography. In some embodiments, Compound 8 can be purified via precipitation. In some embodiments, Compound 8 can be purified via crystallization. In some embodiments, Compound 8 can be purified via recrystallization.

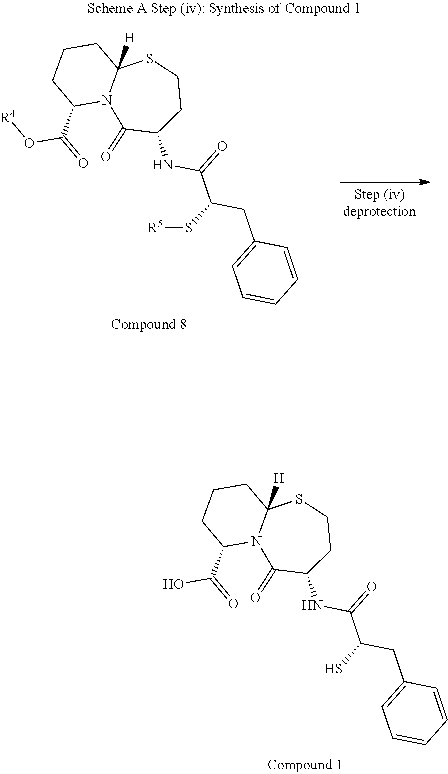

In some embodiments, Compound 1, or a salt thereof, can be prepared from Compound 8, or a salt thereof. In some embodiments, Compound 1, or a pharmaceutically acceptable salt thereof, can be prepared from Compound 8, or a salt thereof. In some embodiments, Compound 8, or a salt thereof, can undergo a deprotection to yield Compound 1, or a salt thereof. In some embodiments, a protecting group can be removed from Compound 8, or a salt thereof, to yield Compound 1, or a salt thereof. In some embodiments, the thiol protecting group can be removed and the ester can be hydrolyzed in the same reaction. In some embodiments, the thiol protecting group can be removed and the ester can be hydrolyzed in different reactions. In some embodiments, the thiol protecting group can be removed and the ester can be hydrolyzed in the same reaction comprising a base. In some embodiments, Compound 8 can be dissolved in a suitable solvent to obtain a solution prior to protecting group removal and ester hydrolysis. In some embodiments, the suitable solvent can be a mixture of solvents and can comprise one or more solvents. In some embodiments, the suitable solvent can be tetrahydrofuran, methyl-tetrahydrofuran, methanol, ethanol, ethylene glycol, acetonitrile, water, or a combination thereof. In some embodiments, the suitable solvent can be tetrahydrofuran, methyl-tetrahydrofuran, methanol, ethanol, water, or a combination thereof. In some embodiments, the suitable solvent can be tetrahydrofuran, methanol, ethanol, ethylene glycol, acetonitrile, water, or a combination thereof. In some embodiments, the suitable solvent can be methanol, ethanol, water, or a combination thereof. In some embodiments, the suitable solvent can be methanol. In some embodiments, the suitable solvent can be methanol and water. In some embodiments, the suitable solvent can be ethanol. In some embodiments, the suitable solvent can be ethanol and water. In some embodiments, the suitable solvent can be tetrahydrofuran. In some embodiments, the suitable solvent can be tetrahydrofuran and water. In some embodiments, the suitable solvent can be methyl-tetrahydrofuran. In some embodiments, the suitable solvent can be methyl-tetrahydrofuran and water. In some embodiments, Step (iv) can comprise about 200 mL to about 50,000 mL of solvent. In some embodiments, Step (iv) can comprise about 500 mL to about 50,000 mL of solvent. In some embodiments, Step (iv) can comprise about 1,000 mL to about 50,000 mL of solvent. In some embodiments, Step (iv) can comprise about 2,000 mL to about 50,000 mL of solvent. In some embodiments, Step (iv) can comprise about 4,000 mL to about 50,000 mL of solvent. In some embodiments, Step (iv) can comprise about 8,000 mL to about 50,000 mL of solvent. In some embodiments, Step (iv) can comprise about 16,000 mL to about 50,000 mL of solvent. In some embodiments, Step (iv) can comprise about 32,000 mL to about 50,000 mL of solvent. In some embodiments, Step (iv) can comprise about 200 mL to about 40,000 mL of solvent. In some embodiments, Step (iv) can comprise about 200 mL to about 20,000 mL of solvent. In some embodiments, Step (iv) can comprise about 200 mL to about 15,000 mL of solvent. In some embodiments, Step (iv) can comprise about 200 mL to about 8,000 mL of solvent. In some embodiments, Step (iv) can comprise about 200 mL to about 4,000 mL of solvent. In some embodiments, Step (iv) can comprise about 200 mL to about 2,000 mL of solvent. In some embodiments, Step (iv) can comprise about 200 mL to about 1,000 mL of solvent. In some embodiments, Step (iv) can comprise about 200 mL to about 500 mL of solvent. In some embodiments, Step (iv) can comprise about 500 mL to about 1,200 mL of solvent. In some embodiments, Step (iv) can comprise about 500 mL to about 1,500 mL of solvent. In some embodiments, Step (iv) can comprise about 1,000 mL to about 3,000 mL of solvent. In some embodiments, Step (iv) can comprise about 2,000 mL to about 4,000 mL of solvent. In some embodiments, Step (iv) can comprise about 4,000 mL to about 6,000 mL of solvent. In some embodiments, Step (iv) can comprise about 5,000 mL to about 7,000 mL of solvent. In some embodiments, Step (iv) can comprise about 6,000 mL to about 8,000 mL of solvent. In some embodiments, Step (iv) can comprise about 7,000 mL to about 9,000 mL of solvent. In some embodiments, Step (iv) can comprise about 8,000 mL to about 10,000 mL of solvent. In some embodiments, Step (iv) can comprise about 9,000 mL to about 11,000 mL of solvent. In some embodiments, Step (iv) can comprise about 10,000 mL to about 12,000 mL of solvent. In some embodiments, Step (iv) can comprise about 11,000 mL to about 13,000 mL of solvent. In some embodiments, Step (iv) can comprise about 12,000 mL to about 14,000 mL of solvent. In some embodiments, Step (iv) can comprise about 13,000 mL to about 15,000 mL of solvent. In some embodiments, Step (iv) can comprise about 14,000 mL to about 16,000 mL of solvent. In some embodiments, Step (iv) can comprise about 15,000 mL to about 17,000 mL of solvent. In some embodiments, the obtained solution of Compound 8 can be heated prior to ester hydrolysis. In some embodiments, the obtained solution of the compound of Compound 8 can be heated to a temperature of about 50° C. to about 80° C. prior to ester hydrolysis. In some embodiments, the obtained solution of Compound 8 can be cooled prior to ester hydrolysis. In some embodiments, the obtained solution of Compound 8 can be cooled to a temperature below about 25° C. prior to ester hydrolysis. In some embodiments, the obtained solution of the compound of Compound 8 can be cooled to a temperature of about −10° C. to about 15° C. prior to ester hydrolysis. In some embodiments, the obtained solution of the compound of Compound 8 can be cooled to a temperature of about −10° C. to about 10° C. prior to ester hydrolysis. In some embodiments, the obtained solution of the compound of Compound 8 can be cooled to a temperature of about −5° C. to about 5° C. prior to ester hydrolysis. In some embodiments, the obtained solution of the compound of Compound 8 can be cooled to a temperature of about 0° C. prior to ester hydrolysis. In some embodiments, the obtained solution of the compound of Compound 8 can be a temperature of about 20° C. to about 25° C. prior to ester hydrolysis. In some embodiments, the obtained solution of the compound of Compound 8 can be a temperature of about 20° C. to about 60° C. prior to ester hydrolysis. In some embodiments, the obtained solution of Compound 8 can be at room temperature prior to ester hydrolysis.

In some embodiments, the thiol deprotection and ester hydrolysis can comprise a metal hydroxide base. In some embodiments, the thiol deprotection and ester hydrolysis can comprise a metal hydroxide base having the formula M-OH; wherein M-OH can be NaOH, KOH, or LiOH, and M⁺ can be Na⁺, K⁺, or Li⁺ respectively. In some embodiments, the thiol deprotection and ester hydrolysis can comprise a metal hydroxide base having the formula M-OH; wherein M-OH can be NaOH, and M⁺ can be Na⁺. In some embodiments, the thiol deprotection and ester hydrolysis can comprise a metal hydroxide base having the formula M-OH; wherein M-OH can be KOH, and M⁺ can be K. In some embodiments, the thiol deprotection and ester hydrolysis can comprise a metal hydroxide base having the formula M-OH; wherein M-OH can be LiGH, and M⁺ can be Li⁺. In some embodiments, the thiol deprotection and ester hydrolysis occurs over about 0.5 to about 16 hours. In some embodiments, the thiol deprotection and ester hydrolysis occurs over about 1 to about 8 hours hour. In some embodiments, the thiol deprotection and ester hydrolysis occurs over about 1 to about 3 hours. In some embodiments, the thiol deprotection and ester hydrolysis occurs over less than about 16 hours. In some embodiments, the thiol deprotection and ester hydrolysis occurs over less than about 8 hours. In some embodiments, the thiol deprotection and ester hydrolysis occurs over less than about 4 hours. In some embodiments, the thiol deprotection and ester hydrolysis occurs over less than about 3 hours. In some embodiments, the thiol deprotection and ester hydrolysis occurs over less than about 2 hours. In some embodiments, the thiol deprotection and ester hydrolysis occurs about 1 to about 2 hours. In some embodiments, the thiol deprotection and ester hydrolysis occurs over about 1 hour. In some embodiments, the thiol deprotection and ester hydrolysis occurs over about 1.5 hours. In some embodiments, the thiol deprotection and ester hydrolysis occurs over less than about 2 hours. In some embodiments, the thiol deprotection and ester hydrolysis occurs over less than about 2.5 hours. In some embodiments, the thiol deprotection and ester hydrolysis occurs over less than about 3 hours. In some embodiments, a shorter thiol deprotection and ester hydrolysis reaction time can reduce epimerization of Compound 1. In some embodiments, epimerization of Compound 1 can be reduced to less than 30%. In some embodiments, epimerization of Compound 1 can be reduced to less than 25%. In some embodiments, epimerization of Compound 1 can be reduced to less than 20%. In some embodiments, epimerization of Compound 1 can be reduced to less than 15%. In some embodiments, epimerization of Compound 1 can be reduced to less than 10%. In some embodiments, epimerization of Compound 1 can be reduced to less than 5%.

In some embodiments, the metal hydroxide base can be added to the solution of Compound 8 as a solution in water. In some embodiments, the concentration of the solution of metal hydroxide base in water can be from about 0.5 M to about 5.0 M. In some embodiments, the metal hydroxide base can be added as a solution in water. In some embodiments, the concentration of the solution of metal hydroxide base in water can be from about 0.5 M to about 1.5 M. In some embodiments, the concentration of the solution of metal hydroxide base in water can be about 0.1 M, about 0.5 M, about 1.0 M, about 2.0 M, or about 5.0 M. In some embodiments, the concentration of the solution of metal hydroxide base in water can be about 1.0 M. In some embodiments, the concentration of the solution of metal hydroxide base in water can be 1.0 M. In some embodiments, the metal hydroxide base can be added to the solution of Compound 8 dropwise. In some embodiments, the metal hydroxide base can be added to the solution of Compound 8 portion wise.

In other embodiments, the ester hydrolysis can comprise an inorganic acid. In some embodiments, the inorganic acid can be hydrochloric acid, sulfuric acid, trifluoroacetic acid, formic acid, or nitric acid. In some embodiments, the resulting acid can be converted to Compound 1 with a suitable base.

In some embodiments, Compound 1, or a salt thereof, can be isolated via precipitation. In some embodiments, Compound 1, or a salt thereof, can be precipitated via acidifying the suitable solvent comprising Compound 1 to about pH 1 to about pH 4. In some embodiments, the solvent can be acidified to about pH 1 to about pH 2. In some embodiments, the solvent can be acidified to about pH 2 to about pH 3. In some embodiments, the solvent can be acidified to about pH 3 to about pH 4. In some embodiments, the solvent can be acidified to about pH 2. In some embodiments, the solvent can be acidified with HCl, trifluoroacetate, or acetic acid. In some embodiments, the solvent can be acidified to about pH 2 with HCl. In some embodiments, Compound 1, or a salt thereof, can be isolated via reverse precipitation wherein the solution comprising Compound 1 is added to an acidic solution. In some embodiments, the acidic solution is an HCl solution, a trifluoroacetate solution, or an acetic acid solution. In some embodiments, the acidic solution is an HCl solution. In some embodiments, Compound 1, or a salt thereof, can be isolated by extraction. In some embodiments, Compound 1, or a salt thereof, can be purified via column chromatography. In some embodiments, Compound 1, or a salt thereof, can be purified via precipitation. In some embodiments, Compound 1, or a salt thereof, can be purified via crystallization. In some embodiments, Compound 1, or a salt thereof can be purified via recrystallization. In some embodiments, Compound 1 can be purified via a trituration. In some embodiments, the trituration can comprise acetonitrile. In some embodiments, the acetonitrile can be heated. In some embodiments, the trituration can comprise refluxing acetonitrile. In some embodiments, the trituration can comprise refluxing acetonitrile, wherein the acetonitrile is refluxed for about 10 minutes to about 24 hours and allowed to cool for about 1 hour to about 24 hours. In some embodiments, the acetonitrile can be refluxed for about 30 minutes. In some embodiments, the acetonitrile can be refluxed for about 40 minutes. In some embodiments, the acetonitrile can be refluxed for about 60 minutes. In some embodiments, the acetonitrile can be refluxed for about 2 hours. In some embodiments, the acetonitrile can be refluxed for about 4 hours. In some embodiments, the acetonitrile can be refluxed for about 8 hours. In some embodiments, the acetonitrile can be refluxed for about 12 hours. In some embodiments, the acetonitrile can be refluxed for about 16 hours. In some embodiments, the acetonitrile can be refluxed for about 20 hours. In some embodiments, the acetonitrile can be refluxed for about 24 hours. In some embodiments, the refluxed acetonitrile can be allowed to cool for about 2 hours. In some embodiments, the refluxed acetonitrile can be allowed to cool for about 4 hours. In some embodiments, the refluxed acetonitrile can be allowed to cool for about 6 hours. In some embodiments, the refluxed acetonitrile can be allowed to cool for about 8 hours. In some embodiments, the refluxed acetonitrile can be allowed to cool for about 12 hours. In some embodiments, the refluxed acetonitrile can be allowed to cool for about 16 hours. In some embodiments, the refluxed acetonitrile can be allowed to cool for about 20 hours. In some embodiments, the refluxed acetonitrile can be allowed to cool for about 24 hours. In some embodiments, the trituration removes the Compound 1 impurities. In some embodiments, the trituration removes the Compound 1 epimer with the structure

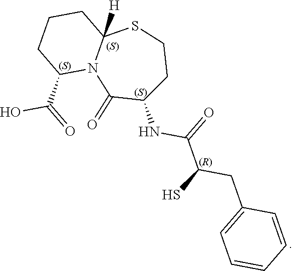

In some embodiments, Compound 1 can be stereochemically enriched. In some embodiments, Compound 1 can be optically pure. In some embodiments, the optical purity of Compound 1 can be greater than about 50% enantiomeric excess. In some embodiments, the optical purity of Compound 1 can be greater than about 60% enantiomeric excess. In some embodiments, the optical purity of Compound 1 can be greater than about 70% enantiomeric excess. In some embodiments, the optical purity of Compound 1 can be greater than about 75% enantiomeric excess. In some embodiments, the optical purity of Compound 1 can be greater than about 80% enantiomeric excess. In some embodiments, the optical purity of Compound 1 can be greater than about 85% enantiomeric excess. In some embodiments, the optical purity of Compound 1 can be greater than about 90% enantiomeric excess. In some embodiments, the optical purity of Compound 1 can be greater than about 95% enantiomeric excess. In some embodiments, the optical purity of Compound 1 can be greater than about 96% enantiomeric excess. In some embodiments, the optical purity of Compound 1 can be greater than about 97% enantiomeric excess. In some embodiments, the optical purity of Compound 1 can be greater than about 98% enantiomeric excess. In some embodiments, the optical purity of Compound 1 can be greater than about 99% enantiomeric excess.

Solvents are categorized into three classes. Class 1 solvents are toxic and are to be avoided. Class 2 solvents are solvents to be limited in use during the manufacture of the therapeutic agent. Class 3 solvents are solvents with low toxic potential and of lower risk to human health. Data for Class 3 solvents indicate that they are less toxic in acute or short-term studies and negative in genotoxicity studies.

Class 1 solvents, which are to be avoided, include: benzene; carbon tetrachloride; 1,2-dichloroethane; 1,1-dichloroethene; and 1,1,1-trichloroethane.

Examples of Class 2 solvents are: acetonitrile, chlorobenzene, chloroform, cumene, cyclohexane, cyclopentyl methyl ether, 1,2-dichloroethene, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, methylisobutyl ketone, N-methylpyrrolidine, nitromethane, pyridine, sulfolane, tetrahydrofuran, tetralin, toluene, 1,1,2-trichloroethene and xylene.

Class 3 solvents, which possess low toxicity, include: acetic acid, acetone, anisole, 1-butanol, 2-butanol, butyl acetate, tert-butyl methyl ether (MTBE), dimethyl sulfoxide, ethanol, ethyl acetate, ethyl ether, ethyl formate, formic acid, heptane, isobutyl acetate, isopropyl acetate, methyl acetate, 3-methyl-1-butanol, methyl ethyl ketone, 2-methyltetrahydrofuran, 2-methyl-1-propanol, pentane, 1-pentanol, 1-propanol, 2-propanol, propyl acetate, and triethylamine.

Residual solvents in active pharmaceutical ingredients (APIs) originate from the manufacture of API. In some cases, the solvents are not completely removed by practical manufacturing techniques. Appropriate selection of the solvent for the synthesis of APIs may enhance the yield, or determine characteristics such as crystal form, purity, and solubility. Therefore, the solvent can be a critical parameter in the synthetic process.

In some embodiments, Compound 1 can comprise an organic solvent(s). In some embodiments, Compound 1 can include a residual amount of an organic solvent(s). In some embodiments, Compound 1 can comprise a residual amount of a Class 3 solvent. In some embodiments, the Class 3 solvent can be selected from the group consisting of acetic acid, acetone, anisole, 1-butanol, 2-butanol, butyl acetate, tert-butyl methyl ether (MTBE), dimethyl sulfoxide, ethanol, ethyl acetate, ethyl ether, ethyl formate, formic acid, heptane, isobutyl acetate, isopropyl acetate, methyl acetate, 3-methyl-1-butanol, methyl ethyl ketone, 2-methyltetrahydrofuran, 2-methyl-1-propanol, pentane, 1-pentanol, 1-propanol, 2-propanol, propyl acetate, and triethylamine. In some embodiments, the Class 3 solvent can be selected from ethyl acetate, isopropyl acetate, tert-butyl methyl ether, heptane, isopropanol, and ethanol.

In some embodiments, Compound 1 can include a detectable amount of an organic solvent. In some embodiments, the organic solvent can be a Class 3 solvent.

In other embodiments, Compound 1 can comprise a detectable amount of solvent that can be less than about 1%, wherein the solvent can be selected from acetone, 1,2-dimethoxyethane, dichloromethane, acetonitrile, ethyl acetate, tetrahydrofuran, 2-methyltetrahydrofuran, methanol, ethanol, heptane, and 2-propanol. In some embodiments, Compound 1 can comprise a detectable amount of solvent that can be selected from the group consisting of dichloromethane, acetonitrile, and 2-methyltetrahydrofuran. In a further embodiment, Compound 1 can comprise a detectable amount of solvent, wherein each solvent can be less than about 5000 ppm. In yet a further embodiment, Compound 1, can comprise a detectable amount of solvent, wherein each solvent can be less than about 5000 ppm, less than about 4000 ppm, less than about 3000 ppm, less than about 2500 ppm, less than about 2000 ppm, less than about 1000 ppm, less than about 500 ppm, or less than about 100 ppm.

### Alternative Preparations of Compound 1

Described herein are alternative methods for the synthesis of Compound 1 as outlined in Scheme B.

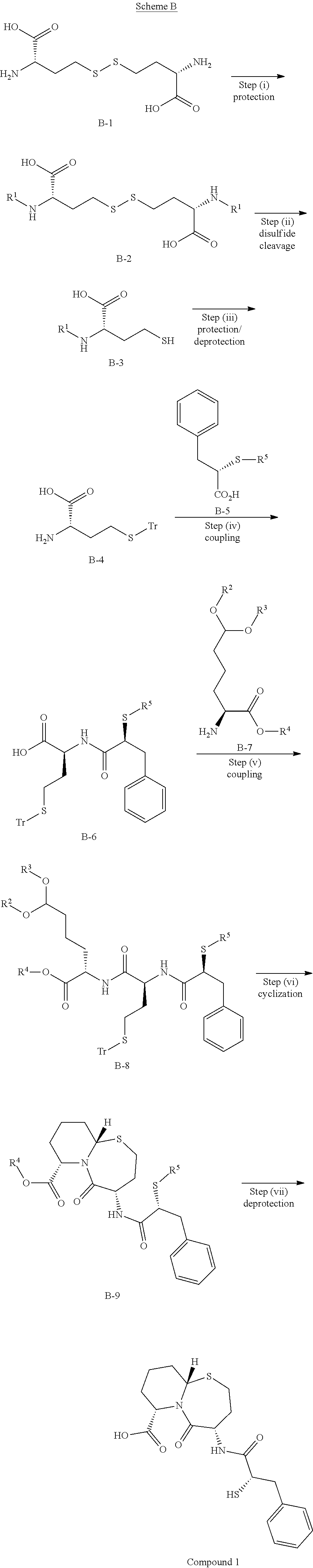

wherein R¹ can be a protecting group;
R² and R³ each can be C₁₋₃ alkyl, or R² and R³ can be taken together to form dioxolane;
R⁴ can be C₁₋₃ alkyl;
R⁵ can be —C(O)—C₁₋₃ alkyl or —C(O)-phenyl; and
Tr can be a trityl group.

In some embodiments, Compound B-1, or a salt thereof, can yield Compound B-2, or a salt thereof, in Step (i). In some embodiments, Compound B-1, or a salt thereof, can be protected with a nitrogen protecting group to form Compound B-2, or a salt thereof, in Step (i). In some embodiments, Compound B-2, or a salt thereof, can undergo disulfide cleavage to yield Compound B-3, or a salt thereof, in Step (ii). In some embodiments, the thiol in Compound B-3, or a salt thereof, can be protected and the protecting group R¹ can be removed to yield Compound B-4 in Step (iii). In some embodiments, Compound B-4, or a salt thereof, and Compound B-5, or a salt thereof, can together yield Compound B-6, or a salt thereof, in Step (iv). In some embodiments, Compound B-4, or a salt thereof, and Compound B-5, or a salt thereof, can together undergo a coupling reaction to yield Compound B-6, or a salt thereof, in Step (iv). In some embodiments, Compound B-4, or a salt thereof, and Compound B-5, or a salt thereof, can together undergo an amide coupling reaction to yield Compound B-6, or a salt thereof, in Step (iv). In some embodiments, Compound B-6, or a salt thereof, and Compound B-7, or a salt thereof, can together yield Compound B-8, or a salt thereof, in Step (v). In some embodiments, Compound B-6, or a salt thereof, and Compound B-7, or a salt thereof, can together undergo a coupling reaction to yield Compound B-8, or a salt thereof, in Step (v). In some embodiments, Compound B-6, or a salt thereof, and Compound B-7, or a salt thereof, can together undergo an amide coupling reaction to yield Compound B-8, or a salt thereof, in Step (v). In some embodiments, Compound B-8, or a salt thereof, can yield Compound B-9, or a salt thereof, in Step (vi). In some embodiments, Compound B-8, or a salt thereof, can undergo cyclization to yield Compound B-9, or a salt thereof, in Step (vi). In some embodiments, the cyclization can be an acid catalyzed cyclization. In some embodiments, Compound B-9, or a salt thereof, can yield Compound 1, or a salt thereof, in Step (vii). In some embodiments, Compound B-9, or a salt thereof, can undergo a deprotection to yield Compound 1, or a salt thereof, in Step (vii). In some embodiments, a protecting group can be removed from Compound B-9, or a salt thereof, to yield Compound 1, or a salt thereof, in Step (vii).

Described herein are alternative methods for the synthesis of Compound 1 as outlined in Scheme C.

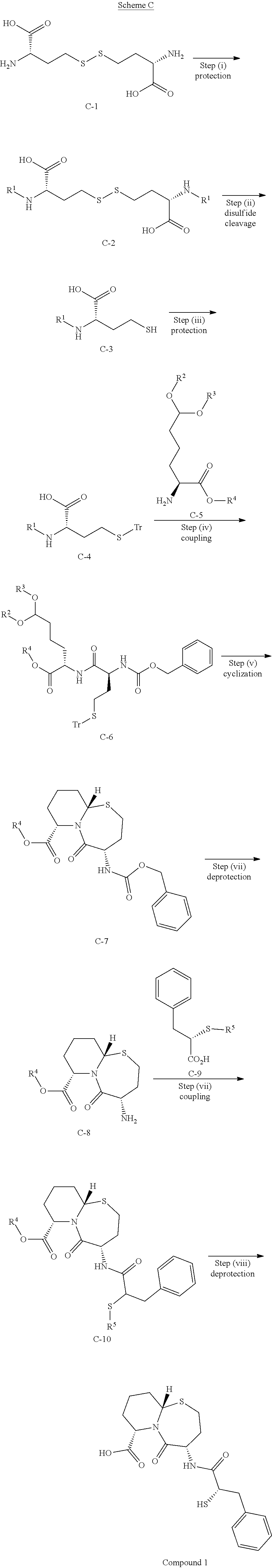

where R¹ can be a protecting group;
R² and R³ each can be C₁₋₃ alkyl, or R² and R³ can be taken together to form dioxolane;
R⁴ can be C₁₋₃ alkyl;
R⁵ can be —C(O)—C₁3 alkyl or —C(O)-phenyl; and
Tr can be a trityl group.

In some embodiments, Compound C-1, or a salt thereof, can yield Compound C-2, or a salt thereof, in Step (i). In some embodiments, Compound C-1, or a salt thereof, can be protected with a nitrogen protecting group to form Compound C-2, or a salt thereof, in Step (i). In some embodiments, Compound C-2, or a salt thereof, can undergo disulfide cleavage to yield Compound C-3, or a salt thereof, in Step (ii). In some embodiments, the thiol in Compound C-3, or a salt thereof, can be protected in to yield Compound C-4 Step (iii). In some embodiments, Compound C-4, or a salt thereof, and Compound C-5, or a salt thereof, can together yield Compound C-6, or a salt thereof, in Step (iv). In some embodiments, Compound C-4, or a salt thereof, and Compound C-5, or a salt thereof, can together undergo a coupling reaction to yield Compound C-6, or a salt thereof, in Step (iv). In some embodiments, Compound C-4, or a salt thereof, and Compound C-5, or a salt thereof, can together undergo an amide coupling reaction to yield Compound C-6, or a salt thereof, in Step (iv). In some embodiments, Compound C-6, or a salt thereof, can yield Compound C-7, or a salt thereof, in Step (v). In some embodiments, Compound C-6, or a salt thereof, can undergo cyclization to yield Compound C-7, or a salt thereof, in Step (v). In some embodiments, the cyclization can be an acid catalyzed cyclization. In some embodiments, Compound C-7, or a salt thereof, can yield Compound C-8, or a salt thereof, in Step (vi). In some embodiments, Compound C-7, or a salt thereof, can undergo a deprotection to yield Compound C-8, or a salt thereof, in Step (vi). In some embodiments, a protecting group can be removed from Compound C-7, or a salt thereof, to yield Compound C-8, or a salt thereof, in Step (vi). In some embodiments, Compound C-8, or a salt thereof, and Compound C-9, or a salt thereof, can together yield Compound C-10, or a salt thereof, in Step (vii). In some embodiments, Compound C-8, or a salt thereof, and Compound C-9, or a salt thereof, can together undergo a coupling reaction to yield Compound C-10, or a salt thereof, in Step (vii). In some embodiments, Compound C-8, or a salt thereof, and Compound C-9, or a salt thereof, can together undergo an amide coupling reaction to yield Compound C-10, or a salt thereof, in Step (vii). In some embodiments, Compound C-10, or a salt thereof, can yield Compound 1, or a salt thereof, in Step (viii). In some embodiments, Compound C-10, or a salt thereof, can undergo a deprotection to yield Compound 1, or a salt thereof, in Step (viii). In some embodiments, a protecting group can be removed from Compound C-10, or a salt thereof, to yield Compound 1, or a salt thereof, in Step (viii).

Described herein are alternative methods for the synthesis of Compound 1 as outlined in Scheme D.

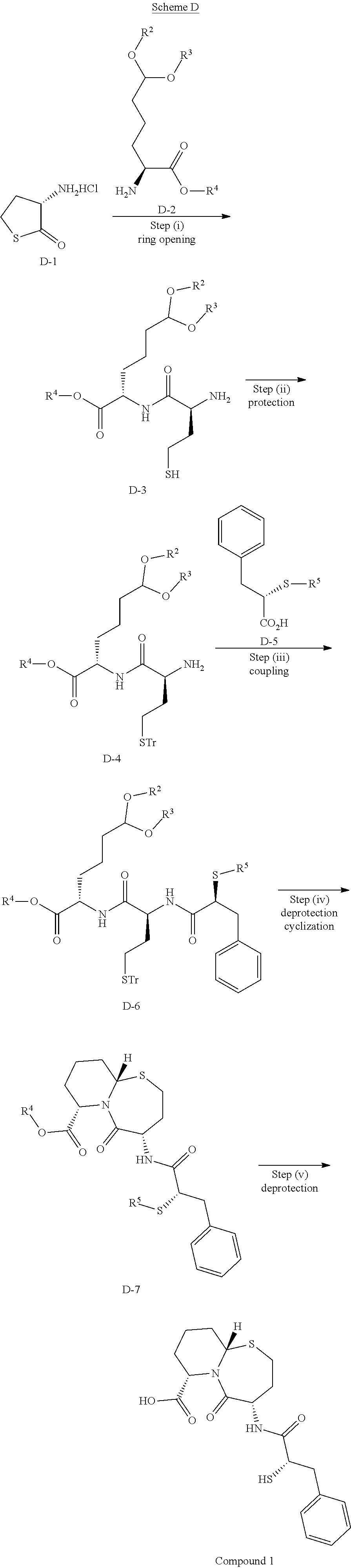

where R² and R³ each can be C₁₋₃ alkyl, or R² and R³ can be taken together to form dioxolane;
R⁴ can be C₁₋₃ alkyl;
R⁵ can be —C(O)—C₁₋₃ alkyl or —C(O)-phenyl; and
Tr can be a trityl group.

In some embodiments, Compound D-1, or a salt thereof, and Compound D-2, or a salt thereof, can together yield Compound D-3, or a salt thereof, in Step (i). In some embodiments, Compound D-1 can undergo a ring opening reaction in the presence of Compound D-2 to yield Compound D-3, or a salt thereof, in Step (i). In some embodiments, Compound D-3, or a salt thereof, can yield Compound D-4, or a salt thereof in Step (ii). In some embodiments, the thiol in Compound D-3, or a salt thereof, can be protected to yield Compound D-4 in Step (ii). In some embodiments, Compound D-4, or a salt thereof, and Compound D-5, or a salt thereof, can together undergo a coupling reaction to yield Compound D-6, or a salt thereof, in Step (iii). In some embodiments, Compound D-4, or a salt thereof, and Compound D-5, or a salt thereof, can together undergo an amide coupling reaction to yield Compound D-6, or a salt thereof, in Step (iii). In some embodiments, Compound D-6, or a salt thereof, can yield Compound D-7, or a salt there of, in Step (iv). In some embodiments, simultaneous deprotection and cyclization of Compound D-6 can yield compound D-7 in Step (iv). In some embodiments, the cyclization can be an acid catalyzed cyclization. In some embodiments, Compound D-7, or a salt thereof, can yield Compound 1, or a salt thereof, in Step (v). In some embodiments, Compound D-7, or a salt thereof, can undergo a deprotection to yield Compound 1, or a salt thereof, in Step (v). In some embodiments, a protecting group can be removed from Compound D-7, or a salt thereof, to yield Compound 1, or a salt thereof, in Step (v).

Described herein are alternative methods for the synthesis of Compound 1 as outlined in Scheme E.

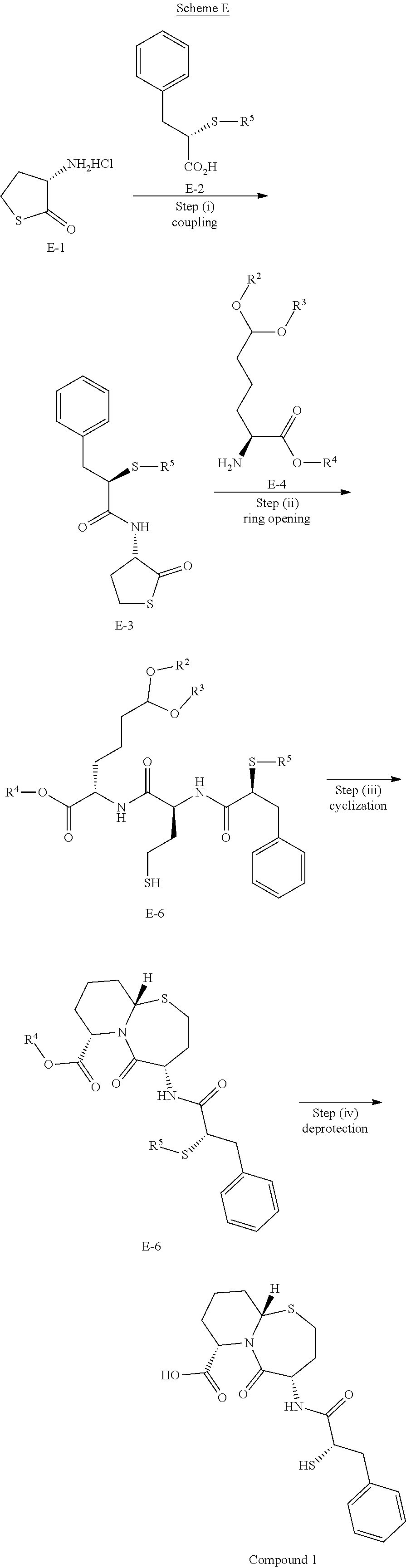

where R² and R³ each can be C₁₋₃alkyl, or R² and R³ can be taken together to form dioxolane;
R⁴ can be C₁₋₃ alkyl; and
R⁵ can be —C(O)—C₁₋₃ alkyl or —C(O)-phenyl.

In some embodiments, Compound E-1, or a salt thereof, and Compound E-2, or a salt thereof, can together undergo a coupling reaction to yield Compound E-3, or a salt thereof, in Step (i). In some embodiments, Compound E-1, or a salt thereof, and Compound E-2, or a salt thereof, can together undergo an amide coupling reaction to yield Compound E-3, or a salt thereof, in Step (i). In some embodiments, Compound E-3, or a salt thereof, and Compound E-4, or a salt thereof, can together yield Compound E-5, or a salt thereof, in Step (ii). In some embodiments, Compound E-3 can undergo a ring opening reaction in the presence of Compound E-4, or a salt thereof, to yield Compound E-5, or a salt thereof, in Step (ii). In some embodiments, Compound E-5, or a salt thereof, can yield Compound E-6, or a salt there of, in Step (iii). In some embodiments, simultaneous deprotection and cyclization of Compound E-5 can yield compound E-6 in Step (iii). In some embodiments, the cyclization can be an acid catalyzed cyclization. In some embodiments, Compound E-6, or a salt thereof, can yield Compound 1, or a salt thereof, in Step (iv). In some embodiments, Compound E-6, or a salt thereof, can undergo a deprotection to yield Compound 1, or a salt thereof, in Step (iv). In some embodiments, a protecting group can be removed from Compound E-6, or a salt thereof, to yield Compound 1, or a salt thereof, in Step (iv).

Described herein are alternative methods for the synthesis of Compound 1 as outlined in Scheme F.

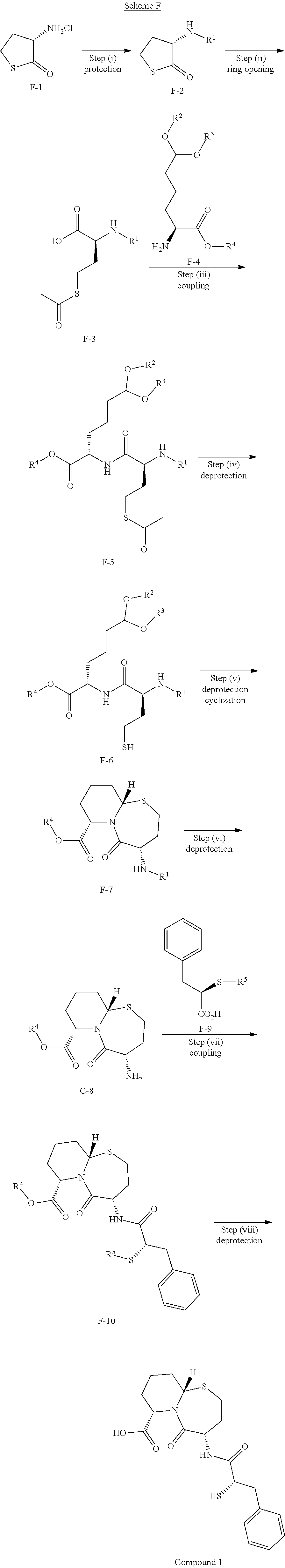

where R¹ can be a protecting group;
R² and R³ each can be C₁₋₃ alkyl, or R² and R³ can be taken together to form dioxolane;
R⁴ can be C₁₋₃ alkyl; and
R⁵ can be —C(O)—C₁₋₃ alkyl or —C(O)-phenyl.

In some embodiments, Compound F-1, or a salt thereof, can yield Compound F-2, or a salt thereof, in Step (i). In some embodiments, Compound F-1, or a salt thereof, can be protected with a nitrogen protecting group to form Compound F-2, or a salt thereof, in Step (i). In some embodiments, Compound F-2, or a salt thereof, can yield Compound F-3, or a salt thereof, in Step (ii). In some embodiments, Compound F-2, or a salt thereof, can undergo a ring opening reaction in the presence of a metal hydroxide to yield Compound F-3, or a salt thereof, in Step (ii). In some embodiments, Compound F-3, or a salt thereof, and Compound F-4, or a salt thereof, can together yield Compound F-5, or a salt thereof in Step (iii). In some embodiments, Compound F-3, or a salt thereof, and Compound F-4, or a salt thereof, can together undergo a coupling reaction to yield Compound F-5, or a salt thereof, in Step (iii). In some embodiments, Compound F-3, or a salt thereof, and Compound F-4, or a salt thereof, can together undergo an amide coupling reaction to yield Compound F-5, or a salt thereof, in Step (iii). In some embodiments, Compound F-5, or a salt thereof, can yield Compound F-6, or a salt thereof, in Step (iv). In some embodiments, the protected thiol of Compound F-5, or a salt thereof, can be deprotected to yield a free thiol in Compound F-6, or a salt thereof, in Step (iv). In some embodiments, Compound F-6, or a salt thereof, can yield Compound F-7, or a salt thereof, in Step (v). In some embodiments, simultaneous deprotection and cyclization of Compound F-6, or a salt thereof, can yield compound F-7, or a salt thereof in Step (v). In some embodiments, the cyclization can be an acid catalyzed cyclization. In some embodiments, Compound F-7, or a salt thereof, can yield Compound F-8, or a salt thereof, in Step (vi). In some embodiments, Compound F-7, or a salt thereof, can undergo a deprotection to yield Compound F-8, or a salt thereof, in Step (vi). In some embodiments, a protecting group can be removed from Compound F-7, or a salt thereof, to yield Compound F-8, or a salt thereof, in Step (vi). In some embodiments, Compound F-8, or a salt thereof, and Compound F-9, or a salt thereof, can together yield Compound F-10, or a salt thereof in Step (vii). In some embodiments, Compound F-8, or a salt thereof, and Compound F-9, or a salt thereof, can together undergo a coupling reaction to yield Compound F-10, or a salt thereof, in Step (vii). In some embodiments, Compound F-8, or a salt thereof, and Compound F-9, or a salt thereof, can together undergo an amide coupling reaction to yield Compound F-10, or a salt thereof, in Step (vii). In some embodiments, Compound F-10, or a salt thereof, can yield Compound 1, or a salt thereof, in Step (viii). In some embodiments, Compound F-10, or a salt thereof, can undergo a deprotection to yield Compound 1, or a salt thereof, in Step (viii). In some embodiments, a protecting group can be removed from Compound F-10, or a salt thereof, to yield Compound 1, or a salt thereof, in Step (viii).

### Isomers/Stereoisomers

In some situations, the compounds described herein possess one or more chiral centers and each center exists in the R configuration or S configuration. The compounds described herein include diastereomeric, enantiomeric, and epimeric forms as well as the corresponding mixtures thereof. In additional embodiments of the compounds and methods provided herein, mixtures of enantiomers and/or diastereomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers, and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent. In some embodiments, provided herein are compounds that are optically enriched.

### Tautomers

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

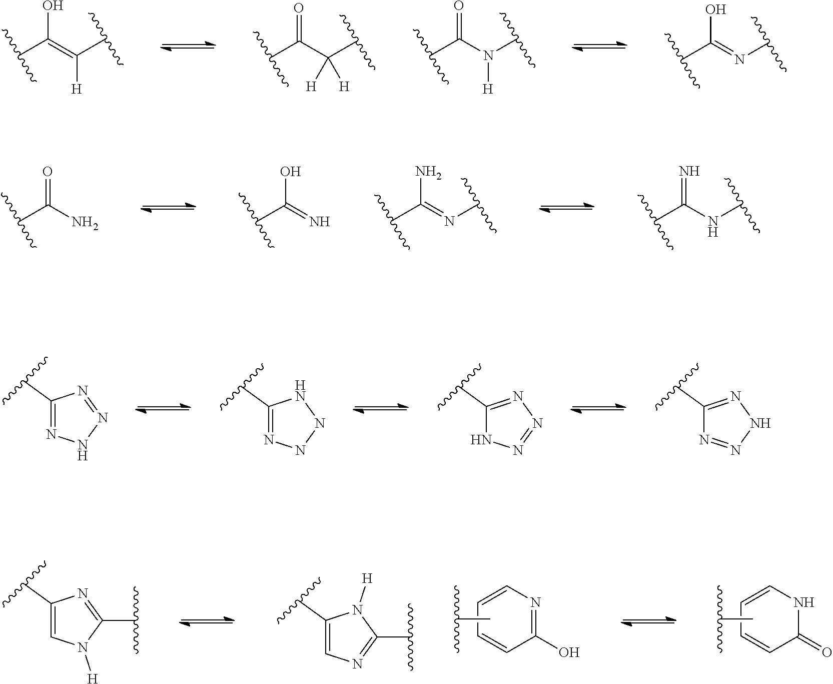

In some instances, the compounds disclosed herein exist in tautomeric forms. The structures of said compounds are illustrated in the one tautomeric form for clarity. The alternative tautomeric forms are expressly included in this disclosure.

### Labeled Compounds

Unless otherwise stated, compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Protium is the most abundant isotope of hydrogen in nature. Enriching for deuterium may afford some therapeutic advantages, such as increased in vivo half-life and/or exposure, or may provide a compound useful for investigating in vivo routes of drug elimination and metabolism.

For example, the compounds described herein may be artificially enriched in one or more particular isotopes. In some embodiments, the compounds described herein may be artificially enriched in one or more isotopes that are not predominantly found in nature. In some embodiments, the compounds described herein may be artificially enriched in one or more isotopes selected from deuterium (²H), tritium (³H), iodine-125 (¹²⁵I) or carbon-14 (¹⁴C). In some embodiments, the compounds described herein are artificially enriched in one or more isotopes selected from ²H, ¹¹C, ¹³C, ¹⁴C, ¹⁵C, ¹²N, ¹³N, ¹⁵N, ¹⁶N, ¹⁶O, ¹²O ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹³¹I, and 125I. In some embodiments, the abundance of the enriched isotopes is independently at least 1%, at least 10%, at least 20%, at least 30%, at least 40%, at least 50%, at least 60%, at least 70%, at least 80%, at least 90%, or 100% by molar.

In some embodiments, the compound is deuterated in at least one position. In some embodiments, the compounds disclosed herein have some or all of the ¹H atoms replaced with ²H atoms.

The methods of synthesis for deuterium-containing compounds are known in the art and include, by way of non-limiting example only, the procedure described in U.S. Pat. Nos. 5,846,514 and 6,334,997, and the following synthetic methods. For example, deuterium substituted compounds may be synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

### Pharmaceutically Acceptable Salts

In some embodiments, the compounds described herein exist as their pharmaceutically acceptable salts.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral acid, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, y-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid, metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts, and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein. Accordingly, one aspect of the present disclosure pertains to hydrates and solvates of compounds of the present disclosure and/or their pharmaceutical acceptable salts, as described herein, that can be isolated and characterized by methods known in the art, such as, thermogravimetric analysis (TGA), TGA-mass spectroscopy, TGA-Infrared spectroscopy, powder X-ray diffraction (PXRD), Karl Fisher titration, high resolution X-ray diffraction, and the like.

### Preparation of the Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH, Inc. (Toronto, Canada), Bionet (Cornwall, U.K.), Chem Service Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN O-19-509618-5; Larock, R. C. “Comprehensive Organic Transformations: A Guide to Functional Group Preparations” 2nd Edition (1999) Wiley-VCH, ISBN: O-471-19031-4; March, J. “Advanced Organic Chemistry: Reactions, Mechanisms, and Structure” 4th Edition (1992) John Wiley & Sons, ISBN: O-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: O-471-93022-9; Solomons, T. W. G. “Organic Chemistry” 7th Edition (2000) John Wiley & Sons, ISBN: O-471-19095-0; Stowell, J. C., “Intermediate Organic Chemistry” 2nd Edition (1993) Wiley-Interscience, ISBN: O-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes, each of which is hereby incorporated by reference.

Specific and analogous reactants are optionally identified through the indices of known chemicals prepared by the Chemical Abstract Service of the American Chemical Society, which are available in most public and university libraries, as well as on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

### Pharmaceutical Compositions

The Compound 1 (including purified Compound 1) described herein, including e.g., pharmaceutically acceptable salt or solvate thereof, can be administered per se as a pure chemical or as a component of a pharmaceutically acceptable formulation.

In another aspect, provided herein are pharmaceutical compositions comprising Compound 1 (including purified Compound 1) described herein, or a pharmaceutically acceptable salt thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), herein incorporated by reference for such disclosure.

In one aspect, the disclosure provides a pharmaceutical composition comprising Compound 1 (including purified Compound 1) described herein, or a pharmaceutically acceptable salt or solvate thereof, and a pharmaceutically acceptable excipient or carrier. In certain embodiments, the Compound 1 as described is substantially pure, in that it contains less than about 10%, less than about 9%, less than about 8%, less than about 7%, less than about 6%, less than about 5%, less than about 4%, less than about 3%, less than about 2%, less than about 1%, less than about 0.5%, or less than about 0.1%, of other organic small molecules, such as unreacted intermediates or synthesis by-products that are created, for example, in one or more of the steps of the synthesis method described herein.

### Examples

### Abbreviations

ACN or MeCN: acetonitrile;
AcOH: acetic acid;
Aq or aq: aqueous;
Boc: tert-butyloxycarbonyl;
Cbz: benzyloxycarbonyl;
DBU: 1,8-Diazabicyclo[5.4.0]undec-7-ene;
DCC: N,N′-Dicyclohexylcarbodiimide;
DCM: dichloromethane;
DIPEA: diisoproylethylamine;
DMAP: 4-dimethylaminopyridine;
DMF: dimethylformamide;
DTT: dithiothreitol;
equiv or eq.: equivalents;
Et: ethyl;
EtOAc: ethyl acetate;
EtOH: ethanol;
Fmoc: fluorenylmethyloxycarbonyl;
h or hr: hour;
hrs: hours;
H¹NMR; proton NMR;
HPLC: high-performance liquid chromatography;
LC-MS or LCMS or LC/MS: liquid chromatography-mass spectrometry;
KOH: potassium hydroxide;
LiOH: lithium hydroxide;
M: molar;
MeCN: acetonitrile;
MEK: methyl ethyl ketone;
Me: methyl;
MeOH: methanol;
Me-THF or methyl THF: 2-methyltetrahydrofuran;
mins or min: minutes;
NaOH: sodium hydroxide;
pNPC: 4-nitrophenyl chloroformate;
PyBOP: benzotriazole-1-yl-oxy-tris-pyrrolidino-phosphonium hexafluorophosphate;
rt or RT: room temperature;
SMEAH: sodium bis(2-methoxyethoxy)aluminium hydride;
T3P: propanephosphonic acid anhydride;
TBME: methyl tert-butyl ether;
TFA: trifluoroacetic acid;
TEA: triethylamine;
THF: tetrahydrofuran;
TMS-I: trimethylsilyl iodide;
Tosyl: toluenesulfonyl;
Trityl: triphenylmethyl;
vol: volume, typically used for reaction volume or ratio of solvents; and
w/w or wt: weight ratio.

The following examples are provided for illustrative purposes only and not to limit the scope of the claims provided herein.

### Analytical Methods:

### Chiral Method for the Determination of the Purity of Compound 1:

System: Waters Acquity UPC2 system with UV detector and QDA detector
Column: Daicel Chiralpak IC-3 (3.0×150 mm; 3 μm)
Mobile Phase A: CO₂
Mobile Phase B: Methanol
Pump Flow: 1.2 mL/Min
UV detection: 212 nm
Injection Volume: 1 μL
Total Run Time: 10 Min
Column Temperature: 40° C.
ABPR: 2000 psi
Mass Detection: MS Scan ES positive and negative
Mass Range: 100-600 Da
Pump Program:

Gradient

| Time | % | % | |
|---|---|---|---|
| (Min) | A | B | Curve |
| Initial | 98 | 2 | Initial |
| 6 | 60 | 40 | 6 |
| 9 | 60 | 40 | 6 |
| 10 | 98 | 2 | 11 |

### Non-Chiral Method for the Determination of the Purity of Compound 1:

System: Agilent 1290 series with UV detector and HP 6130 MSD mass detector
Column: Zorbax RRHD SB C8 (2.1×50 mm; 1.8 μm)
Mobile Phase A: 0.05% Trifluoroacetic acid (aq)
Mobile Phase B: Acetonitrile
Pump Flow: 0.6 mL/Min
UV Detection: 215 nm
Injection Volume: 0.2 μL
Run Time: 4.0 Min
Post Time: 1.0 Min
Column Temperature: 35° C.
Mass Detection: API-ES positive and negative
Mass Range: 80-1500 Da
Pump Program:

Gradient

| Time | % | % |
|---|---|---|
| (Min) | A | B |
| 0.5 | 90 | 10 |
| 2.0 | 10 | 90 |
| 3.0 | 10 | 90 |

### Mass-Only Method for Compound ]

System: Agilent 1260 series with UV detector, ELSD 1260 detector and Agilent 6120 mass detector
Mobile Phase A: Ammonium acetate (10 mM); Water/Methanol/Acetonitrile (900:60:40)
Mobile Phase B: Ammonium acetate (10 mM); Water/Methanol/Acetonitrile (100:540:360)
Pump Flow: 0.2 mL/Min
Injection Volume: 10.0 μL
Run Time: 3.0 Min
Mass Detection: API-ES positive and negative
Mass Range: 100-1200 Da
Pump Program:

Gradient

| Time | % | % |
|---|---|---|
| (Min) | A | B |
| 0.0 | 50 | 50 |
| 1.5 | 0 | 100 |
| 3.0 | 0 | 100 |

### Example 1: Synthesis of (2S,2'S)-4,4′-disulfanediylbis(2-(((benzyloxy)carbonyl)amino)butanoic acid) (Compound 2-Cbz)

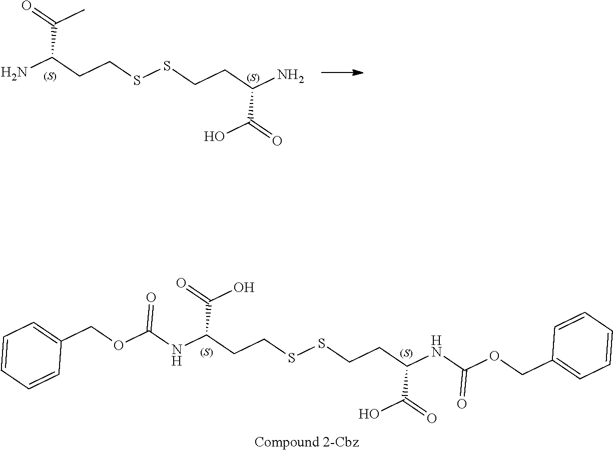

In a round bottom flask (2S,2'S)-4,4′-disulfanediylbis(2-aminobutanoic acid) (20.8 g, 1 Eq, 77.5 mmol) was suspended in THF (92.2 g, 104 mL, 16.5 Eq, 1.28 mol) and cooled to 4° C. A 20% solution of NaOH (13.0 g, 4.2 Eq, 326 mmol) in water (65.6 g, 65.6 mL, 47 Eq, 3.64 mol) was slowly added, the mixture became an orange solution (pH=14). Then, benzyl chloroformate (29.3 g, 24.5 mL, 97% Wt, 2.15 Eq, 167 mmol) was added dropwise. The mixture was stirred from 4° C. to room temperature for a total of 3 h. The reaction was diluted with TBME and washed with water, the water phase was washed 2× 150 mL TBME. The water phase (pH=8) was acidified with HCl 6M to pH=3. The product separated as an oil. The water phase was extracted with 2×250 mL EtOAc, then washed with brine, dried over sodium sulfate and concentrated, co-evaporated twice with toluene, to give the product as a light-yellow sticky solid. Product (2S,2'S)-4,4′-disulfanediylbis(2-(((benzyloxy)carbonyl)amino)butanoic acid) (39.5 g, 73.6 mmol, 95%) was obtained in about 70% purity (215 nm) and used as such in the next step.

Choice of amine protecting group impacts product yields as summarized in Table 1.

TABLE 1

| Protecting | Product |
|---|---|
| group | yield |
| Formy1 | 76% |
| Cbz | 95% |
| Trifluoroacetyl | 100% |

### Example 1a: Large Scale Synthesis of (2S,2'S)-4,4′-disulfanediylbis(2-(((benzyloxy)carbonyl)amino)butanoic acid) (Compound 2-Cbz)

In a 3 neck round bottom flask, with nitrogen inlet and thermometer, (2S,2'S)-4,4′-disulfanediylbis(2-aminobutanoic acid) (60 g, 1 Eq, 0.22 mol) was suspended in THF (0.27 kg, 0.30 L, 16.5 Eq, 3.7 mol) and cooled to 4° C. A 20% solution of NaOH (38 g, 4.2 Eq, 0.94 mol) in water (0.19 kg, 0.19 L, 47 Eq, 11 mol) was slowly added over 20 min, the mixture became an orange solution (pH=14), no exotherm observed. Then, benzyl chloroformate (85 g, 71 mL, 97% wt, 2.15 Eq, 0.48 mol) was added dropwise over 20 min. The mixture was stirred from 4° C. to room temperature for 3 h then checked by HPLC. The reaction was stirred for a total of 7 h, then diluted with TBME and washed with water. The water phase was washed 2× TBME, then both organic phase and water phase were checked by HPLC.
Total volume org phase: 1.0 L (containing mainly impurities)
Total volume aq phase: 700 mL (containing mainly product)

The water phase (pH=8) containing the product was acidified with HCl 6M to pH=3. The product separated as a brown oil/suspension on the bottom. The water phase was extracted first with iPrOAc (500 mL). The organic phase was diluted with EtOAc (150 mL) then washed with brine. The first water phase was washed with EtOac (2×250 mL), and both organic phases were joined for a total of 1 L organic phase. The organic phase was left standing at room temperature overnight and the product crystallized out of the organic phase as a white solid that was filtered, washed with TBME (200 mL) and dried on air (55 g, 46%, as a white solid).

The solid (55 g) was co-evaporated with toluene (250 mL) and concentrated to dryness at 50° C. to give 52 g of product used as such in the next step.

The mother liquor was concentrated, then dissolved in 300 mL EtOAc and cooled to 0° C. TBME (200 mL) was added over 10 min. Some solid precipitated was observed and 100 mL of heptane was added at rt. The mixture stirred for 2 h and was filtered. The product was observed in the mother liquor and separated from the precipitated solid. The mother liquor was concentrated to yield the product as a yellow foam (52 g, 43%) Product was obtained in 86% yield as two batches, 52 g of clean solid, and 52 g of mother liquor, less clean and used as such in the next step.

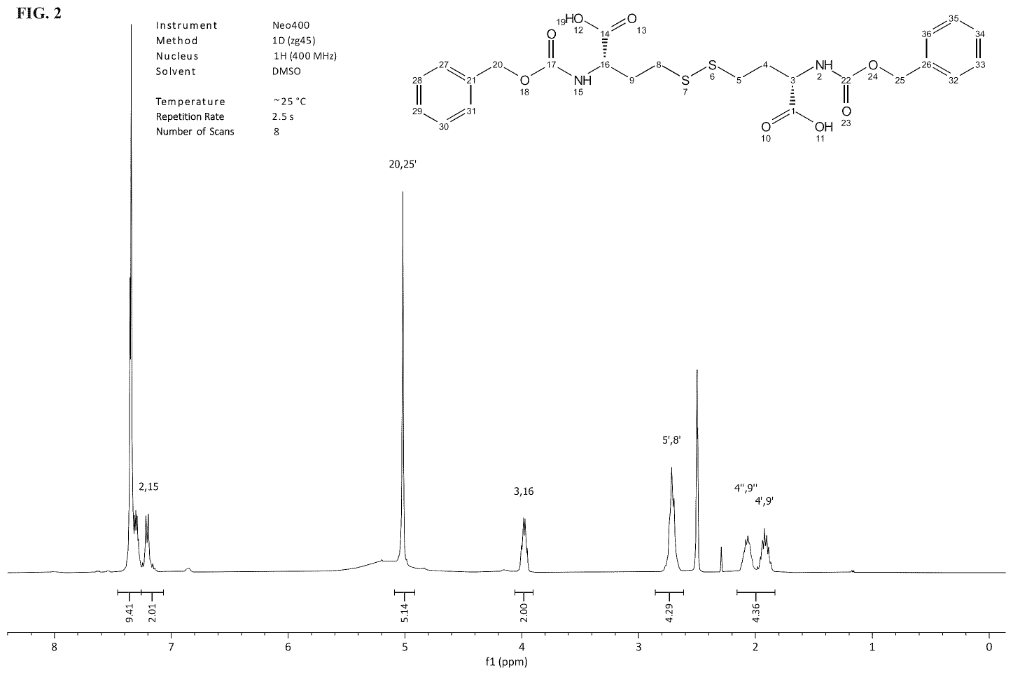

H¹NMR for Compound 2-Cbz obtained from the solid is shown in FIG. 2.

MS: m/z=537.2 [M+H]⁺.

### Example 2: Synthesis of methyl (S)-2-amino-5-(1,3-dioxolan-2-yl)pentanoate hydrochloride (Compound 3a-HCl)

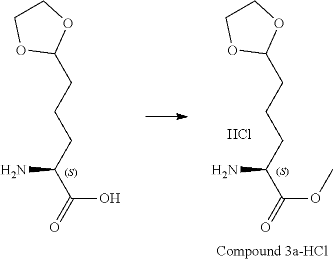

In a 3-neck round bottom flask (S)-2-amino-5-(1,3-dioxolan-2-yl)pentanoic acid (20.4 g, 1 Eq, 108 mmol) was suspended in methanol (415 g, 523 mL, 120 Eq, 12.9 mol) and cooled to −4° C. (salt and ice), then SOCl₂ (25.7 g, 15.7 mL, 2 Eq, 216 mmol) was added dropwise and the mixture stirred overnight at room temperature. The reaction mixture was concentrated at 40° C. and stripped 3× toluene to obtain a white solid that was used as such in the next step. Product methyl (S)-2-amino-5-(1,3-dioxolan-2-yl)pentanoate hydrochloride (25.8 g, 108 mmol) was obtained in quantitative yield as a white solid.

### Example 2a: Large Scale Synthesis of methyl (S)-2-amino-5-(1,3-dioxolan-2-yl)pentanoate hydrochloride (Compound 3a-HCl)

In a 3 neck, 3 L round bottom flask with nitrogen inlet and thermometer, (S)-2-amino-5-(1,3-dioxolan-2-yl)pentanoic acid (60 g, 1 Eq, 0.32 mol) was suspended in methanol (1.2 kg, 1.5 L, 120 Eq, 38 mol) and cooled to −4° C. (salt and ice) then SOCl₂ (75 g, 46 mL, 2 Eq, 0.63 mol) was added dropwise over 20 min and the mixture stirred overnight from −4° C. to room temperature. The reaction mixture was concentrated at 40° C., then co-evaporated with toluene (3×400 mL), to give a white solid. It was discovered that traces of water or SOCl₂ in the crude product lead to compound degradation, indicating the importance of controlled co-evaporation with toluene. The solid was stored in the freezer for one week. The solid was defrosted and analyzed by NMR in water. Product was obtained in quantitative yield and used as such in the next step.

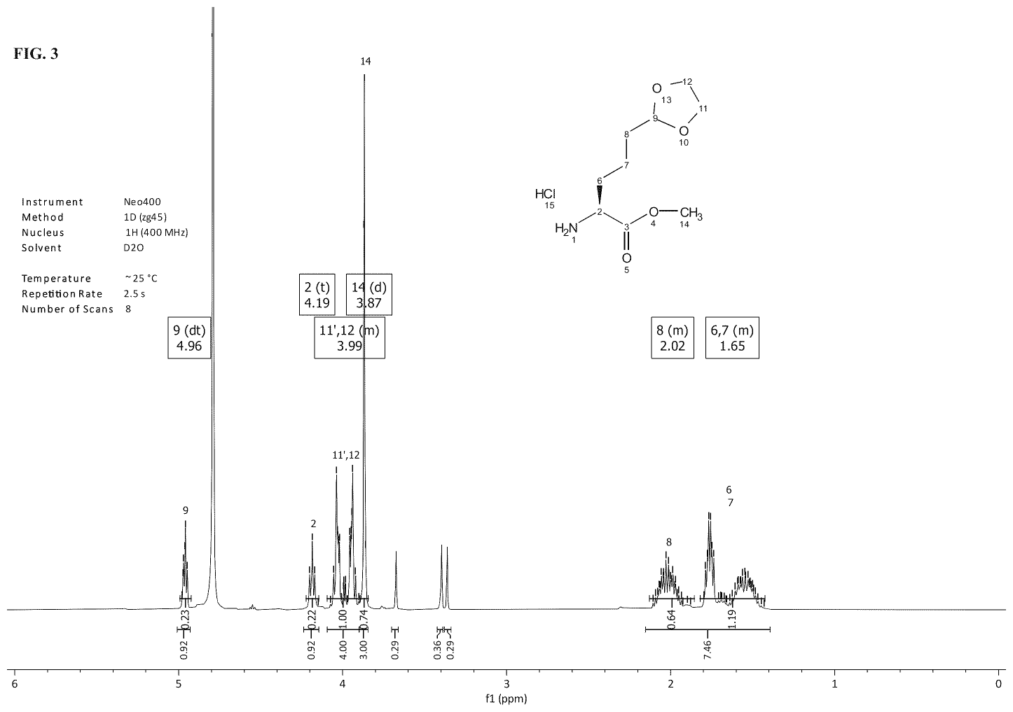

H¹NMR for Compound 3a-HCl is shown in FIG. 3.

### Example 3: Synthesis of dimethyl 2,2′-(((2S,2'S)-4,4′-disulfanediylbis(2-(((benzyloxy)carbonyl)amino)butanoyl))bis(azanediyl))(2S,2'S)-bis(5-(1,3-dioxolan-2-yl)pentanoate) (Compound 4a)

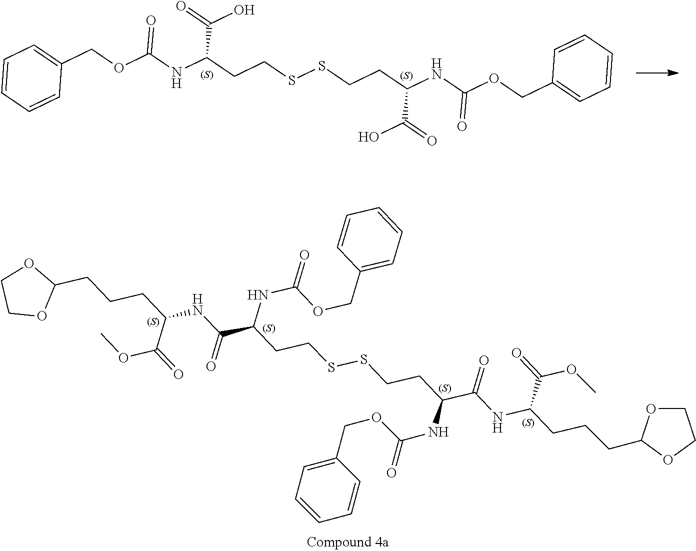

In a round bottom flask (2S,2'S)-4,4′-disulfanediylbis(2-(((benzyloxy)carbonyl)amino)butanoic acid) (24.8 g, 1 Eq, 46.2 mmol) was dissolved in EtOAc (228 g, 253 mL, 56 Eq, 2.59 mol) (solution) and added to a suspension of (S)-5-(1,3-dioxolan-2-yl)-1-methoxy-1-oxopentan-2-aminium chloride (25.5 g, 2.3 Eq, 106 mmol) in acetonitrile (114 g, 145 mL, 60 Eq, 2.77 mol). The mixture (suspension) was cooled to 0° C. and DIPEA (29.9 g, 40.3 mL, 5 Eq, 231 mmol) was added (dropwise over 10 min, still a suspension, easy to stir). Then T₃P (70.6 g, 64.8 mL, 50% Wt in EtOAc, 2.4 Eq, 111 mmol) was added dropwise in 15 min (still a suspension). The reaction was stirred from 0° C. to room temperature over 2 h. The mixture was worked up by washing with sat. NaHCO₃. The water phase was washed with EtOAc, dried over sodium sulfate and concentrated. The crude was dissolved in 170 mL DCM at 30° C. and treated with TBME (about 80 mL at room temperature) and the product precipitated as a white solid. This gave 39 g of Compound 4a in 93% yield and 70% purity (215 nm).

Purity of the precipitate by both ¹H-NMR and HPLC is comparable to the purity of the material when purified by column chromatography; however, the yield on this step increased remarkably from 35% (isolation via chromatography) to 93% (isolation via precipitation) due to previously unrecognized compound instability on silica.

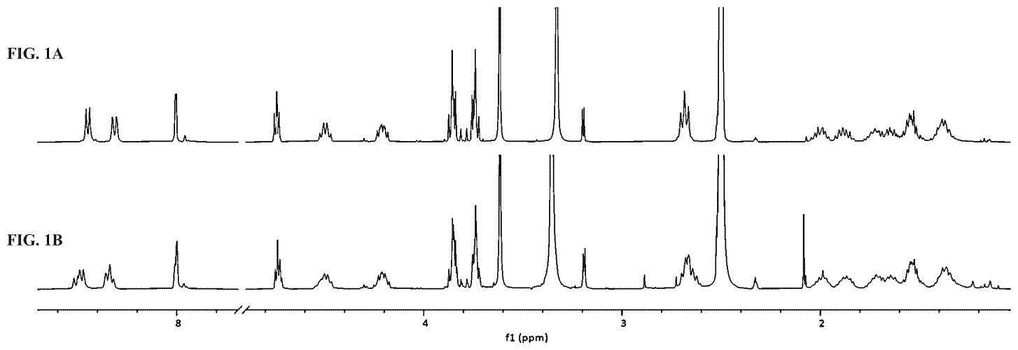

Choice of coupling reagent impacts product yields and isomer formation as summarized in Table 2 and FIG. 1A and FIG. 1B.

TABLE 2

| Protecting | Coupling | | | | Product | |
|---|---|---|---|---|---|---|
| group | Agent | Solvent | Base | Amine | formation | Yield |
| Formyl | DCC | THF/DMF | DIPEA | 3a-HCl | — | — |
| Formy1 | DCC | DMF | TEA/DMAP | 3a-HCl | — | — |
| Formyl | pNPC | DMF | TEA/DMAP | 3a-HCl | Observed | 26%* |
| Formyl | pNPC | DMF | TEA/DMAP | 3a-HCl | Observed | 38%* |
| Formyl | T3P | DMF | TEA | 3a-HCl | Observed | 30%* |
| Cbz | T3P | DMF | DIPEA | 3a-HCl | Observed | 35%* |
| Cbz | T3P | EtOAc | DIPEA | 3a-HCl | — | — |
| Cbz | T3P | EtOAc/MeCN | DIPEA | 3a-HCl | Observed | 93%** |

*indicates isolation and purification by chromatography;
**indicates isolation and purification by precipitation.

¹H-NMR of the batch of Compound 4a-Formyl coming from the reaction with pNPC was compared to ¹H-NMR of the batch of Compound 4a-Formyl coming from the reaction with T3P and a clear difference was observed. The batch of Compound 4a-Formyl coming from the reaction with T3P (FIG. 1A) was cleaner, and signals that in the pNPC batch (FIG. 1B) were multiplets (8.5 ppm, 2.7 ppm) or triplets (8.35) became well defined doublets or triplets. From this observation it was concluded that the reaction with pNPC gave racemization in the phase of activation of the acid starting material.

### Example 3a: Large Scale Synthesis of dimethyl 2,2′-(((2S,2'S)-4,4′-disulfanediylbis(2-(((benzyloxy)carbonyl)amino)butanoyl))bis(azanediyl))(2S,2'S)-bis(5-(1,3-dioxolan-2-yl)pentanoate) (Compound 4a)

In a round bottom flask with nitrogen inlet and thermometer, (2S,2'S)-4,4′-disulfanediylbis(2-(((benzyloxy) carbonyl)amino)butanoic acid) (69.6 g, 1 Eq, 130 mmol) was suspended in EtOAc (640 g, 710 mL, 56 Eq, 7.26 mol) and added to a suspension of (S)-5-(1,3-dioxolan-2-yl)-1-methoxy-1-oxopentan-2-aminium chloride (71.5 g, 2.3 Eq, 298 mmol) in acetonitrile (319 g, 406 mL, 60 Eq, 7.78 mol). The mixture (suspension) was cooled to −5° C. and DIPEA (83.8 g, 113 mL, 5 Eq, 649 mmol) was added dropwise over 10 min. Then T₃P (198 g, 182 mL, 50% Wt, 2.4 Eq, 311 mmol) was added dropwise over 30 min. The temperature was allowed to slowly rise to room temperature. The mixture was worked up by diluting with EtOAc (200 mL) and washing with sat. NaHCO₃ (2×300 mL). The water phase was washed EtOAc (200 mL), dried over sodium sulfate and concentrated (112 g, 95% yield). The crude was used as such in the next step.

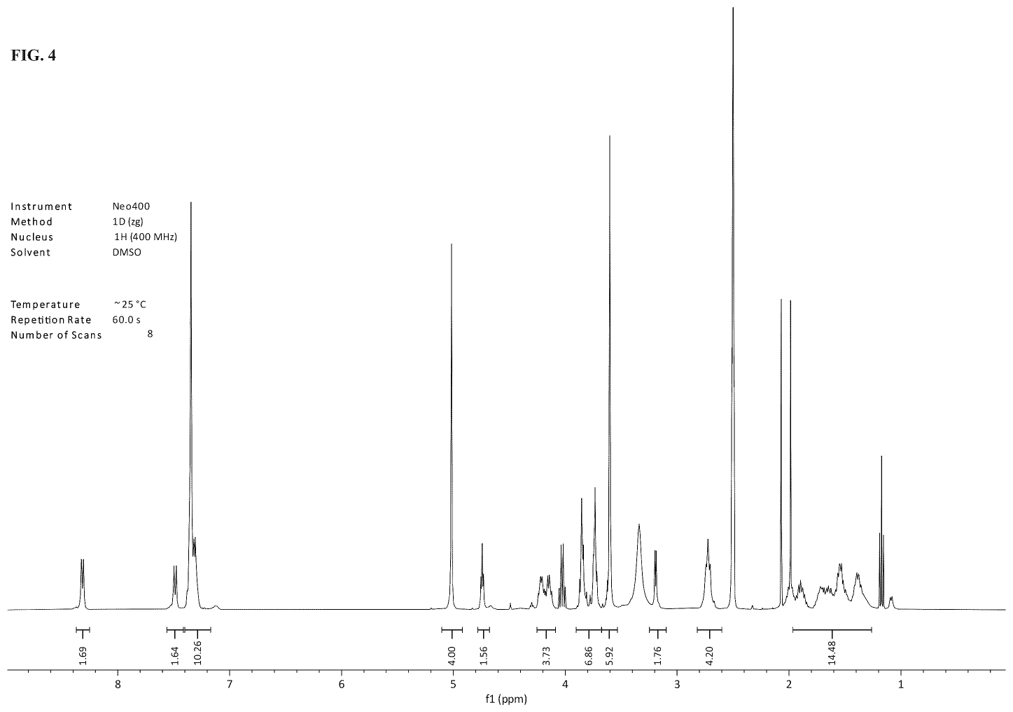

H¹NMR for Compound 4a is shown in FIG. 4.

MS: m/z=907.5 [M+H]⁺.

### Example 4: Synthesis of methyl (4S,7S,10aS)-4-(((benzyloxy)carbonyl)amino)-5-oxooctahydro-7H-pyrido[2,1-b][1,3]thiazepine-7-carboxylate

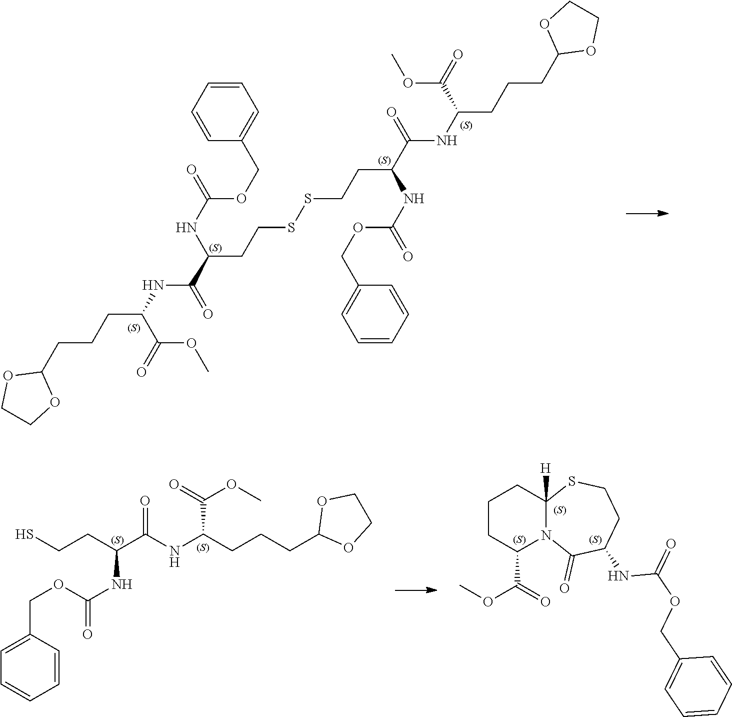

Step 1: dimethyl 2,2′-(((2S,2'S)-4,4′-disulfanediylbis(2-(((benzyloxy)carbonyl)amino)butanoyl))bis(azanediyl))(2S,2'S)-bis(5-(1,3-dioxolan-2-yl)pentanoate) (Compound 4a) (20.0 g, 1 Eq, 22.0 mmol) was dissolved in methyl acetate (474 g, 508 mL, 290 Eq, 6.39 mol) (previously degassed by bubbling N₂). The mixture was cooled to −10° C. DTT (6.80 g, 2 Eq, 44.1 mmol) was added and then sodium methoxide (596 mg, 2.04 mL, 5.4 molar, 0.5 Eq, 11.0 mmol) and the reaction stirred for 1h at −10° C. to give the intermediate methyl (S)-2-((S)-2-(((benzyloxy)carbonyl)amino)-4-mercaptobutanamido)-5-(1,3-dioxolan-2-yl)pentanoate (Observed mass=513 corresponding to M+59). The reaction was warmed to room temperature and washed with 200 mL H₂SO₄ 0.01 M and the water phase back extracted with EtOAc (aq 1 pH=7). The organic phase was then washed with brine. The organic phase was dried over sodium sulfate and concentrated at 36° C. The crude mixture was analyzed by HPLC and ¹H-NMR.

Step 2: The crude (25 g) was dissolved in acetonitrile (262 g, 334 mL, 290 Eq, 6.39 mol) and TFA (5.03 g, 3.40 mL, 2 Eq, 44.1 mmol) and water (4.77 g, 4.77 mL, 12 Eq, 265 mmol) were added at room temperature. The mixture was stirred overnight. Additional TFA (2.51 g, 1.70 mL, 1 Eq, 22.0 mmol) and water (2.38 g, 2.38 mL, 6 Eq, 132 mmol) were added and the reaction stirred for 2 h. The reaction was concentrated to a reduced volume, then diluted with EtOAc and washed with bicarbonate, the organic phase was dried over sodium sulfate and concentrated. The crude mixture (23 g) was isolated as a yellowish oil and analyzed by HPLC and ¹H-NMR. The crude was dissolved in DCM and injected into a 330 g silica column and eluted with a gradient from 0 to 40% of EtOAc in heptane to give the desired product as a colorless oil (7.1 g, 18 mmol, 41%).

Choice of protecting group impacts product yield in Step 2 as summarized in Table 3. Disulfide cleavage and cyclization were carried out as described in Example 4 except that the protecting group was varied as indicated in Table 3.

TABLE 3

| Protecting | Product | |
|---|---|---|
| group | formation | Yield |
| Formyl | Observed | 27% |
| Trifluoroacetyl | Observed | 10% |
| Cbz | Observed | 41% |

Unexpectedly, presence of water in the reaction and choice of acid impacts product yield in Step 2 as described in Table 4. Amberlyst and TFA are listed below as example acids; however, the transformation depicted in Example 4 is not limited to these acids and other acids are contemplated.

TABLE 4

| Protecting | Solvent water | Product | |
|---|---|---|---|
| group | content/Acid | formation | Yield |
| Formyl | Anhydrous/Amberlyst | — | — |
| Formyl | Wet/Amberlyst | Observed | — |
| Formyl | Wet/TFA | Observed | 27% |

### Example 4a: Large Scale Synthesis of methyl (4S,7S,10aS)-4-(((benzyloxy)carbonyl)amino)-5-oxooctahydro-7H-pyrido[2,1-b][1,3]thiazepine-7-carboxylate

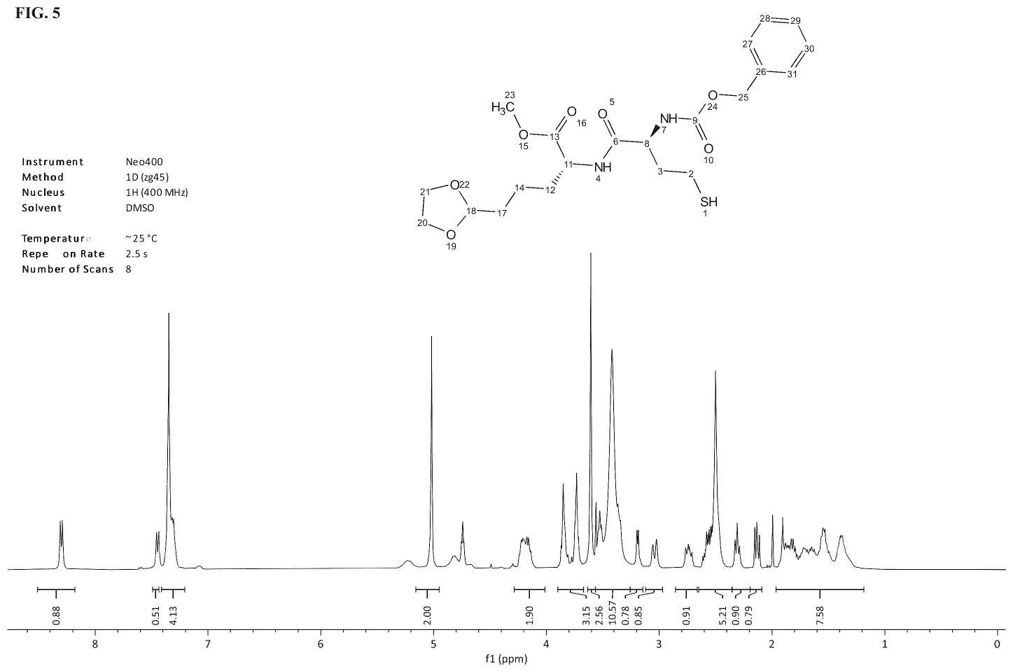

Step 1: In a round bottom flask with nitrogen inlet and thermometer, dimethyl 2,2′-(((2S,2'S)-4,4′-disulfanediylbis(2-(((benzyloxy)carbonyl)amino)butanoyl))bis(azanediyl))(2S,2'S)-bis(5-(1,3-dioxolan-2-yl)pentanoate) (111 g, 1 Eq, 122 mmol) was suspended in methyl acetate (2.09 kg, 2.24 L, 230 Eq, 28.1 mol) (previously degassed by bubbling N₂). The mixture was cooled to −10° C. DTT (37.7 g, 2 Eq, 245 mmol) was added and then sodium methoxide (3.31 g, 11.3 mL, 5.4 molar, 0.5 Eq, 61.2 mmol) and the reaction stirred for 2 h at −8° C. After concluding the reaction was finished by HPCL, the reaction was allowed to warm to 5° C. A solution of sulfuric acid (20.8 g, 11.3 mL, 95% wt, 1.65 Eq, 202 mmol) in water (1.96 kg, 1.96 L, 890 Eq, 109 mol) was prepared (0.1 M). The reaction was washed with 2×1 L H₂SO₄ 0.01 M and the water phase back extracted with MeOAc (300 mL) (aq 1 pH=7). The organic phases were then washed with brine, dried over sodium sulfate, and concentrated at 36° C. The crude mixture (yellowish solid) was analyzed by ¹H-NMR and then stored in the freezer for 72 hours. The crude was defrosted and checked by HPLC before proceeding, no decomposition observed. H¹NMR for the intermediate methyl (S)-2-((S)-2-(((benzyloxy)carbonyl)amino)-4-mercaptobutanamido)-5-(1,3-dioxolan-2-yl)pentanoate is shown in FIG. 5.

Step 2: The crude mixture (yellowish solid) was dissolved in acetonitrile (1.46 kg, 1.85 L, 290 Eq, 35.5 mol) and TFA (41.9 g, 28.3 mL, 3 Eq, 367 mmol) and water (44.1 g, 44.1 mL, 20 Eq, 2.45 mol) were added at 10° C. temperature. The cold bath was removed and mixture was stirred overnight at rt. The reaction was concentrated to reduced volume, then diluted with EtOAc and washed with bicarbonate (2×400 mL, the first aq phase was still acidic, the second aq phase had pH around 8), the water phase was back extracted with EtOAc, the organic phases were washed with brine, dried over sodium sulfate and concentrated at 37° C.

The crude (120 g) was dissolved in DCM and injected into a 1.6 Kg silica column and eluted with a gradient from 0 to 50% of iPrOAc in heptane. Collected: from 1:45 h till 2:10 h (51 g, colorless oil, 53%).

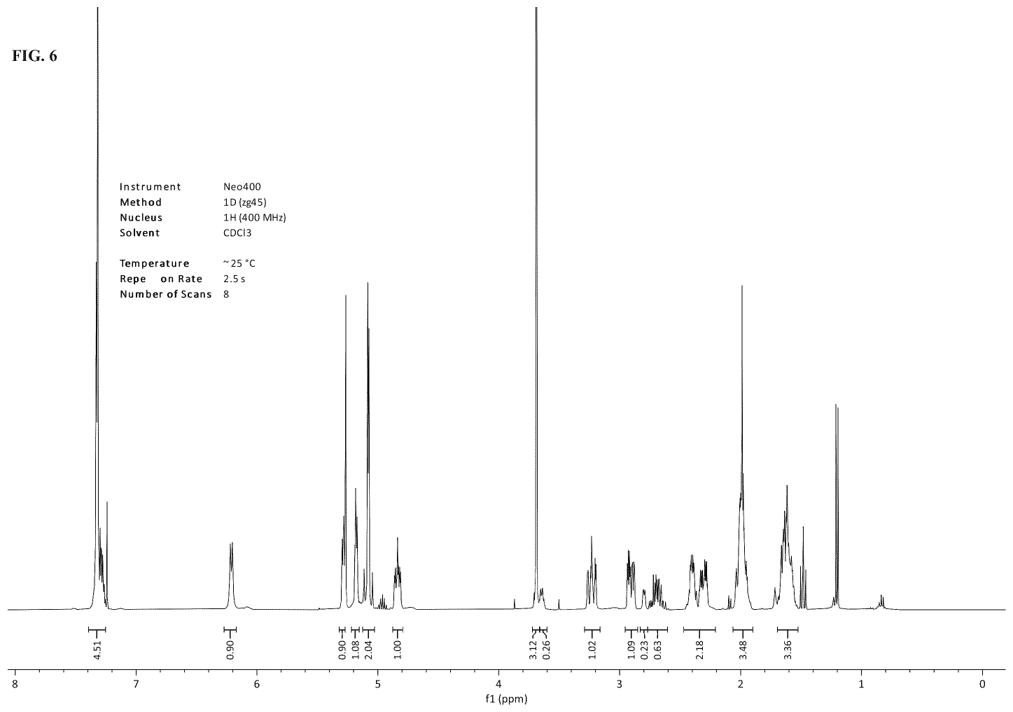

H¹NMR for methyl (4S,7S,10aS)-4-(((benzyloxy)carbonyl)amino)-5-oxooctahydro-7H-pyrido[2,1-b][1,3]thiazepine-7-carboxylate is shown in FIG. 6.

MS: m/z=393.2 [M+H]⁺.

### Example 5: Synthesis of methyl (4S,7S,10aS)-4-amino-5-oxooctahydro-7H-pyrido[2,1-b][1,3]thiazepine-7-carboxylate

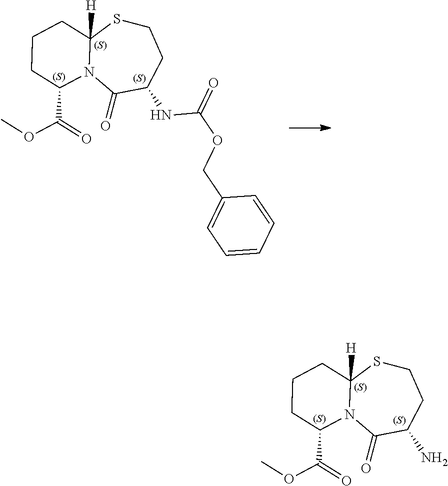

In a round bottom flask methyl (4S,7S,10aS)-4-(((benzyloxy)carbonyl)amino)-5-oxooctahydro-7H-pyrido[2,1-b][1,3]thiazepine-7-carboxylate (7.10 g, 1 Eq, 18.0 mmol) was dissolved in DCM (95.0 g, 72.0 mL, 62 Eq, 1.10 mol) and TMS-I (4.70 g, 3.20 mL, 1.3 Eq, 24.0 mmol) was added. The reaction was stirred for 2 h and checked by HPLC. The reaction was concentrated and then re-dissolved in EtOAc and washed with 1M HCL. The product went to the water phase, the water phase was washed with EtOAc. The water phase was basified to pH 10 with 4M NaOH and saturated with NaCl, then extracted 3× DCM for a total of 400 ml DCM. The organic DCM phase was dried over sodium sulfate and concentrated to give the product as a colorless oil (4.30 g, 17.0 mmol, 92%).

Choice of protecting group impacts product yields as summarized in Table 5.

TABLE 5

| Protecting | Product | |
|---|---|---|
| group | formation | Yield |
| Formyl | Observed | 45% |
| Cbz | Observed | 92% |

### Example 5a: Large Scale Synthesis of methyl (4S,7S,10aS)-4-amino-5-oxooctahydro-7H-pyrido[2,1-b][1,3]thiazepine-7-carboxylate

In a round bottom flask methyl (4S,7S,10aS)-4-(((benzyloxy)carbonyl)amino)-5-oxooctahydro-7H-pyrido[2,1-b][1,3]thiazepine-7-carboxylate (46 g, 1 eq, 0.12 mol) was dissolved in DCM (0.62 kg, 0.47 L, 62 Eq, 7.3 mol) and TMS-I (30 g, 21 mL, 1.3 Eq, 0.15 mol) was added. The reaction was stirred for 1 h and then checked by HPLC (MeOH). The reaction was concentrated and then re-dissolved in isopropyl acetate (0.24 kg, 0.28 L, 20 Eq, 2.3 mol) and washed with 1 M HCL (26 g, 0.70 L, 1.0 molar, 6 eq, 0.70 mol). The product went to the water phase, the water phase was washed with iPrOAc. Both phases were checked, the product is in water. The water phase was basified to pH 10 with 4 M NaOH (26 g, 0.16 L, 4.0 molar, 5.5 Eq, 0.64 mol) and saturated with NaCl, then extracted 3× DCM (500 mL total). The water phase was checked by HPLC: a small amount of product still in the water layer, the water layer was extracted 2×200 mL DCM, the joined organic phases were dried over sodium sulfate and concentrated. The product was obtained in 80% yield (24.32 g) as a brown oil and used as such in the next step. In some embodiments, the product may be isolated via precipitation as an HCl or HI salt.

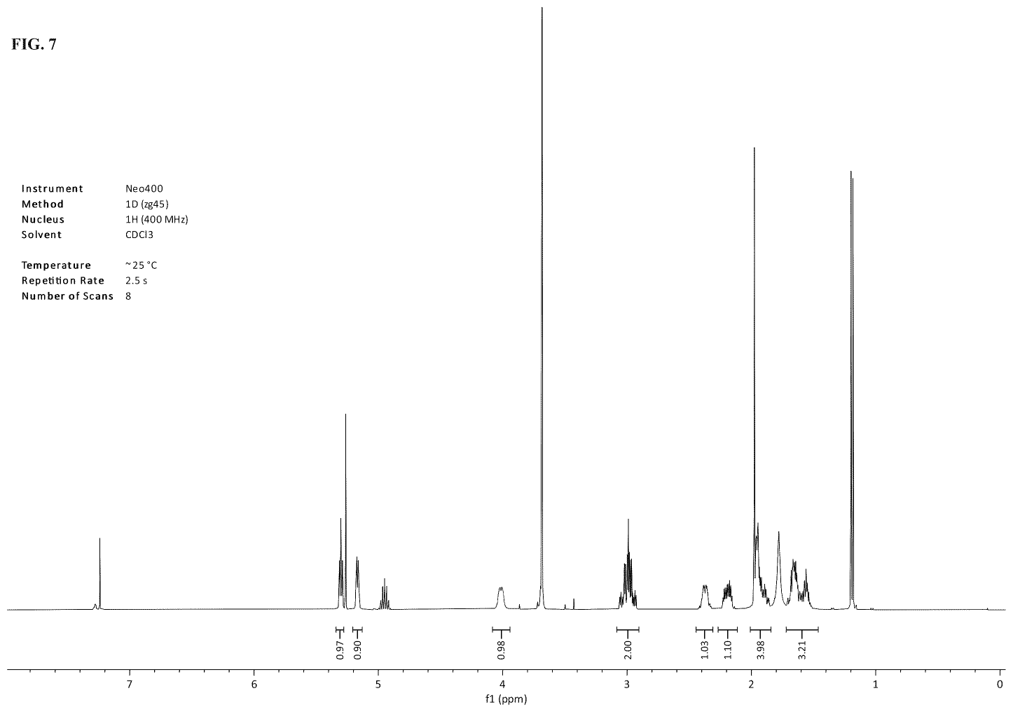

H¹NMR for methyl (4S,7S,10aS)-4-amino-5-oxooctahydro-7H-pyrido[2,1-b][1,3]thiazepine-7-carboxylate is shown in FIG. 7.

MS: m/z=259.2 [M+H]⁺.

### Example 6: Synthesis of (S)-2-(acetylthio)-3-phenylpropanoic acid

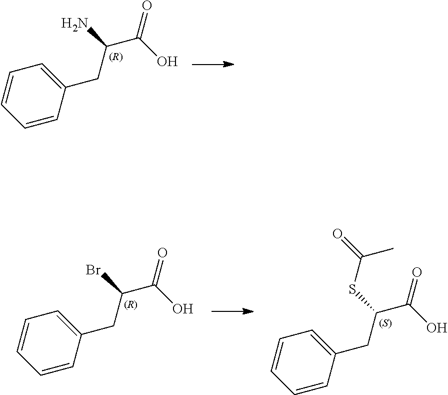

### Step 1: (R)-2-Bromo-3-Phenylpropanoic Acid

In a round bottom flask D-phenylalanine (24.0 g, 1 Eq, 145 mmol) was dissolved in 120 ml water and potassium bromide (58.8 g, 3.4 Eq, 494 mmol) and HBr (53.9 g, 36.2 mL, 48% Wt, 2.2 Eq, 320 mmol) were added. The mixture was cooled on an ice/salt bath to −6.5 to −2.5° C. and sodium nitrite (12.5 g, 1.25 Eq, 182 mmol) was added in portions, under a stream of nitrogen gas. After 40 h at 21° C., the mixture was purged with nitrogen gas, extracted with TBME (3×). The combined organic layers were washed with brine, filtered, and evaporated to afford the title compound as a yellow oil, crude yield 33.3 g (100%). The crude product was used as such for the next step.

### Step 2: (S)-2-(acetylthio)-3-phenylpropanoic acid

In a round bottom flask cesium carbonate (70.4 g, 1.5 Eq, 216 mmol) was dissolved in methanol (500 mL) and cooled to 4° C. Thioacetic acid (16.4 g, 15.5 mL, 1.5 Eq, 216 mmol) was added dropwise and the mixture stirred for 18 h, then methanol was evaporated. The mixture was suspended in DMF (100 mL) and a solution of (R)-2-bromo-3-phenylpropanoic acid (33.0 g, 1 Eq, 144 mmol) in DMF (80 mL), was added dropwise. The temperature went from 21 to 34° C. The mixture was stirred for 18 h at 21° C. The mixture was poured into 500 mL of 1 N HCl, the product was extracted with EtOAc (3×), the organic phase was washed with water (4×) and brine, dried over sodium sulphate, filtered and concentrated to give the product as a pale brown oil (30.5 g, quant. crude). The product (20 g) was purified by column chromatography (330 g silica gel, gradient heptane+2% AcOH: EtOAc 0 to 35%), to give the product as a yellow foam (13 g, 65% yield, 95% ee).

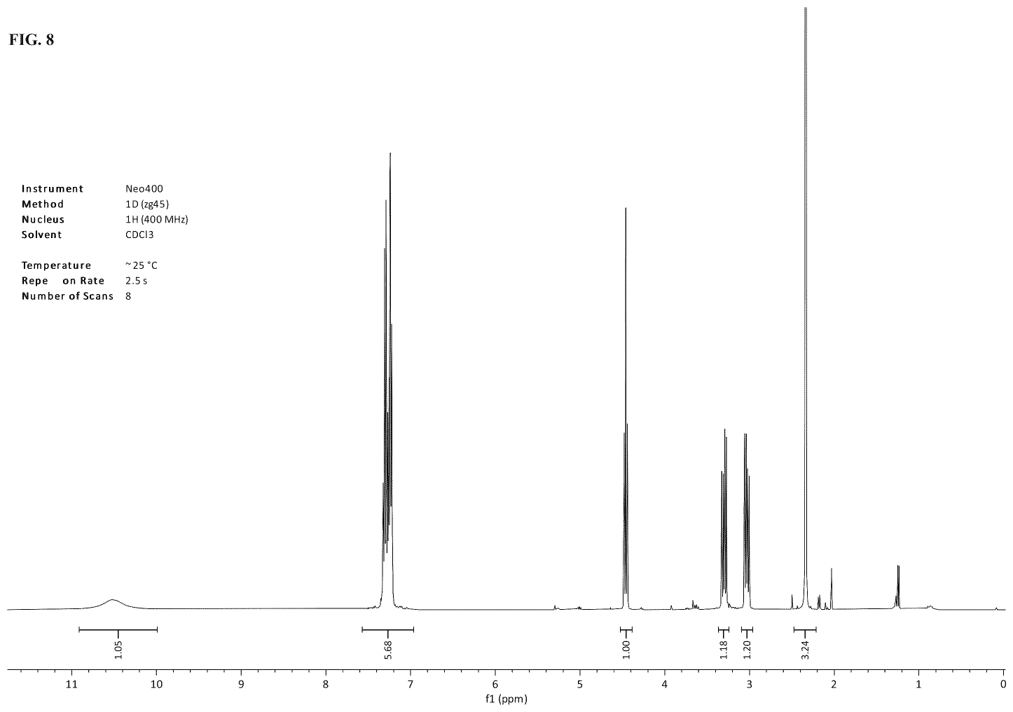

H¹NMR for (S)-2-(acetylthio)-3-phenylpropanoic acid is shown in FIG. 8.

MS: m/z=224.99 [M+H]⁺.

### Example 7: Synthesis of methyl (4S,7S,10aS)-4-((S)-2-(acetylthio)-3-phenylpropanamido)-5-oxooctahydro-7H-pyrido[2,1-b][1,3]thiazepine-7-carboxylate)

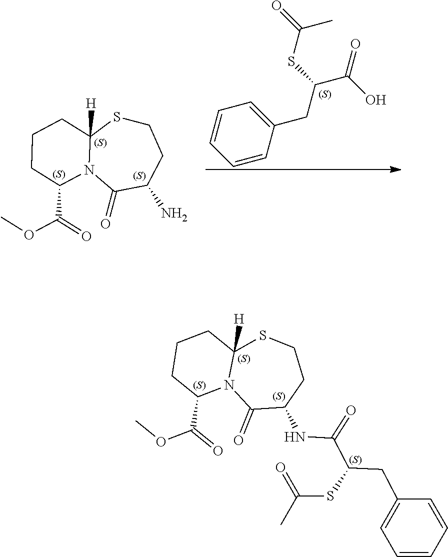

In a round bottom flask (S)-2-(acetylthio)-3-phenylpropanoic acid (4.1 g, 1.1 Eq, 18 mmol) was dissolved in DCM (54.0 g, 41.0 mL, 38 Eq, 0.63 mol) at 0° C. and methyl (4S,7S,10aS)-4-amino-5-oxooctahydro-7H-pyrido[2,1-b][1,3]thiazepine-7-carboxylate (4.30 g, 1 Eq, 17.0 mmol), TEA (1.70 g, 2.30 mL, 1 Eq, 17.0 mmol) and PyBOP (9.50 g, 1.1 Eq, 18.0 mmol) were added. The reaction was stirred from 0° C. to room temperature. After a total of 3 h the reaction was concentrated, the residue dissolved in EtOAc, washed with NaHCO₃ sat solution, dried over Na₂SO₄ and concentrated. This gave 17.0 g of crude as a yellow oil. The crude was dissolved in DCM and purified by column chromatography (330 g silica gel, gradient heptane: EtOAc from 0 to 30%) to give the product as a white solid (6.40 g, 14.0 mmol, 83% yield) with 98% purity (215 nm).

### Example 7a: Large Scale Synthesis of methyl (4S,7S,10aS)-4-((S)-2-(acetylthio)-3-phenylpropanamido)-5-oxooctahydro-7H-pyrido[2,1-b][1,3]thiazepine-7-carboxylate)

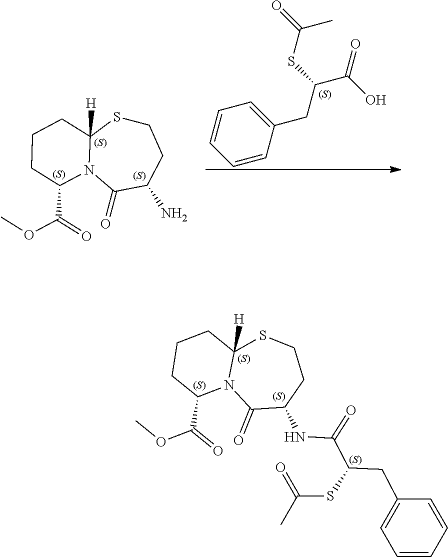

In a round bottom flask (S)-2-(acetylthio)-3-phenylpropanoic acid (23.2 g, 1.1 Eq, 103 mmol) was dissolved in DCM (304 g, 230 mL, 38 Eq, 3.57 mol) and cooled to 0° C. (ice bath, external). To the solution were sequentially added methyl (4S,7S,10aS)-4-amino-5-oxooctahydro-7H-pyrido[2,1-b][1,3]thiazepine-7-carboxylate (24.3 g, 1 Eq, 94.1 mmol), TEA (10.5 g, 14.4 mL, 1.1 Eq, 103 mmol) and PyBOP (53.8 g, 1.1 Eq, 103 mmol). The reaction was stirred from 0° C. to room temperature for 3 hr and then concentrated. The residue was dissolved in iPrOAc, washed with NaHCO₃ sat solution, dried over sodium sulfate and concentrated. The crude (80 g), was dissolved in DCM and purified by column chromatography on 800 g silica column eluting with hept: iPrOAc 0 to 50%. Collected: 1:05 h- 1:20 h. The product was isolated as a colorless oil still containing some DCM and some iPrOAc. The product was concentrated at the rotavapor further. This gave the desired product (35.9 g, 77.3 mmol, 82% yield) as a sticky colorless oil which partially solidified during time. Chiral analysis shows a 97:3 diastereoisomeric mixture.

The tail of the ISCO peak was also collected resulting into a 1:1 mixture of the two diastereomers also containing another unknown impurity.

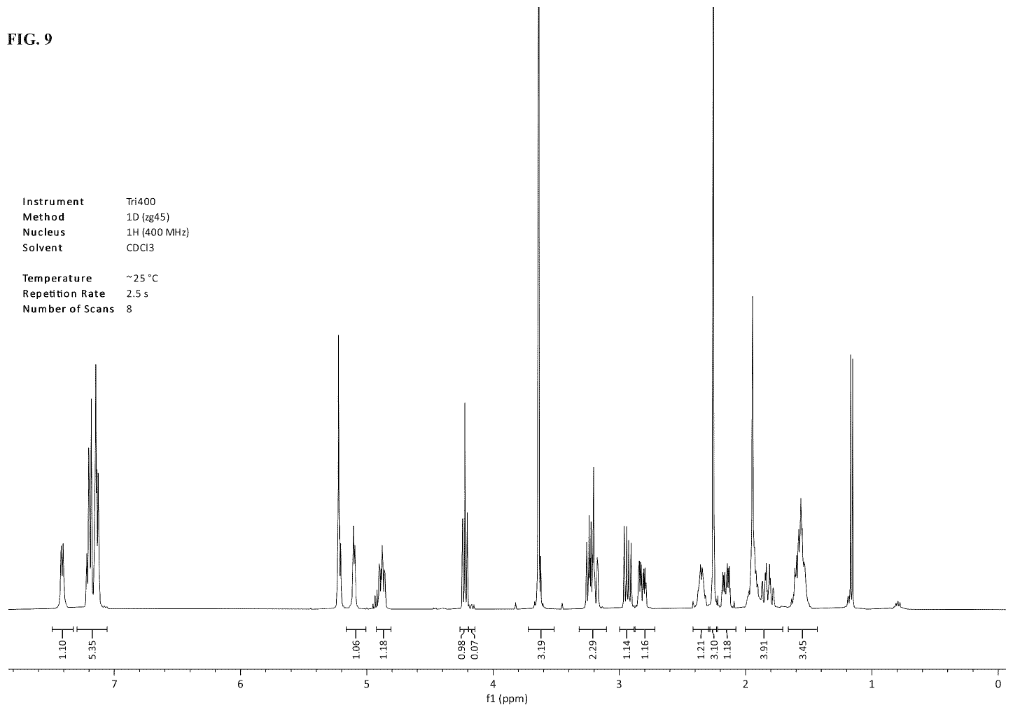

H¹NMR for methyl (4S,7S,10aS)-4-((S)-2-(acetylthio)-3-phenylpropanamido)-5-oxooctahydro-7H-pyrido[2,1-b][1,3]thiazepine-7-carboxylate is shown in FIG. 9.

MS: m/z=465.2 [M+H]⁺.

### Example 8: Synthesis of (4S,7S,10aS)-4-((S)-2-mercapto-3-phenylpropanamido)-5-oxooctahydro-7H-pyrido[2,1-b][1,3]thiazepine-7-carboxylic acid (Compound 1)

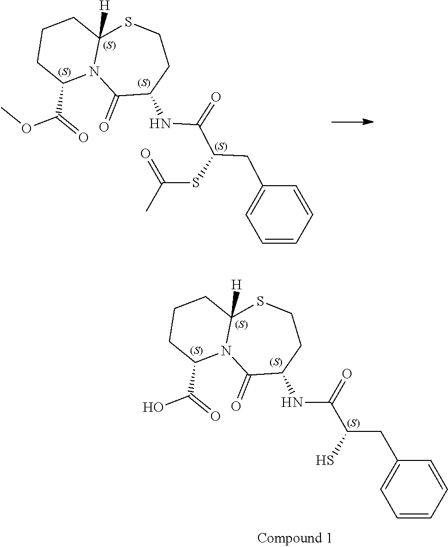

A flask was charged with methyl (4S,7S,10aS)-4-((S)-2-(acetylthio)-3-phenylpropanamido)-5-oxooctahydro-7H-pyrido[2,1-b][1,3]thiazepine-7-carboxylate (295 mg, 1 Eq, 635 gmol) in MeOH (2.44 g, 3.08 mL, 120 Eq, 76.2 mmol) and cooled to 0° C., then a solution of NaOH 1N (0.18 g, 4.44 mL, 1 molar, 7 Eq, 4.44 mmol) was added and the reaction stirred overnight at room temperature. HCl 6 N was slowly added to the mixture until a precipitate formed and pH 2 was reached. The solid was stirred for 30 min then filtered, washed with water and TBME, dried on a filter and then using a rotavapor. The product Compound 1 was isolated as a white solid (4S,7S,10aS)-4-((S)-2-mercapto-3-phenylpropanamido)-5-oxooctahydro-7H-pyrido[2,1-b][1,3]thiazepine-7-carboxylic acid (167 mg, 409 gmol, 64.4%) with 97% purity (215 nm, achiral) and 89% purity (212 nm, chiral).

### Example 8a: Large Scale Synthesis of (4S,7S,10aS)-4-((S)-2-mercapto-3-phenylpropanamido)-5-oxooctahydro-7H-pyrido[2,1-b][1,3]thiazepine-7-carboxylic acid (Compound 1)

methyl (4S,7S,10aS)-4-((S)-2-(acetylthio)-3-phenylpropanamido)-5-oxooctahydro-7H-pyrido[2,1-b][1,3]thiazepine-7-carboxylate (20.6 g, 1 Eq, 44.3 mmol) was dissolved in THF (192 g, 216 mL, 60 Eq, 2.66 mol) and degasses for 30 min, then while keeping bubbling N₂ inside the solution, at 22° C., lithium hydroxide monohydrate (9.30 g, 222 mL, 1.0 molar, 5 Eq, 222 mmol) was added (poured from a 250 mL Erlenmeyer to the open flask of the reaction mixture with funnel) in about 5 min and the temperature reached 28° C. The reaction was checked by HPLC (sample diluted in MeOH) at the end of the addition (20% conversion) and after 45 min (95% conversion). After a total of 1 h 15 min the reaction was checked again (100% conversion) and after a total of 1 h 30 min the reaction was worked up. The reaction was acidified with HCl (9.70 g, 53.2 mL, 5.0 molar, 6 Eq, 266 mmol) in water till pH˜2 (pH paper, during the addition temperature went from 22° C. to 28° C.). 200 mL MeTHF was added and the phases separated. The water phase was back-extracted with 100 mL MeTHF, then the joined organic phases were washed with brine (150 mL) dried over sodium sulfate and concentrated under reduced pressure. Total amount of organic phase˜500 mL. Total amount of water phase˜500 mL. Both aqueous phase and crude product were checked by HPLC: No product observed in aqueous phase; 14% isomerization observed by NMR on crude; 8% isomerization according to UPC. The product was obtained as a white solid (17.3 g, 96% yield). The crude mixture were suspended in 15 vol MeCN (ultrapure for HPLC, 260 mL) refluxed for 2 h and cooled for 4 h. The suspension was filtered on a glass filter, the mother liquor concentrated, the solid dried on filter for 30 min and at the rotavapor at 43° C. for 30 min. This gave the product as a white solid (13.4 g, 32.8 mmol, 74% yield) in 99.75% chiral purity. The mother liquor was also isolated (3.3 g) as a 60: 40 mixture of Iso-Omapatrilat (Iso-Compound 1): Omapatrilat (Compound 1).

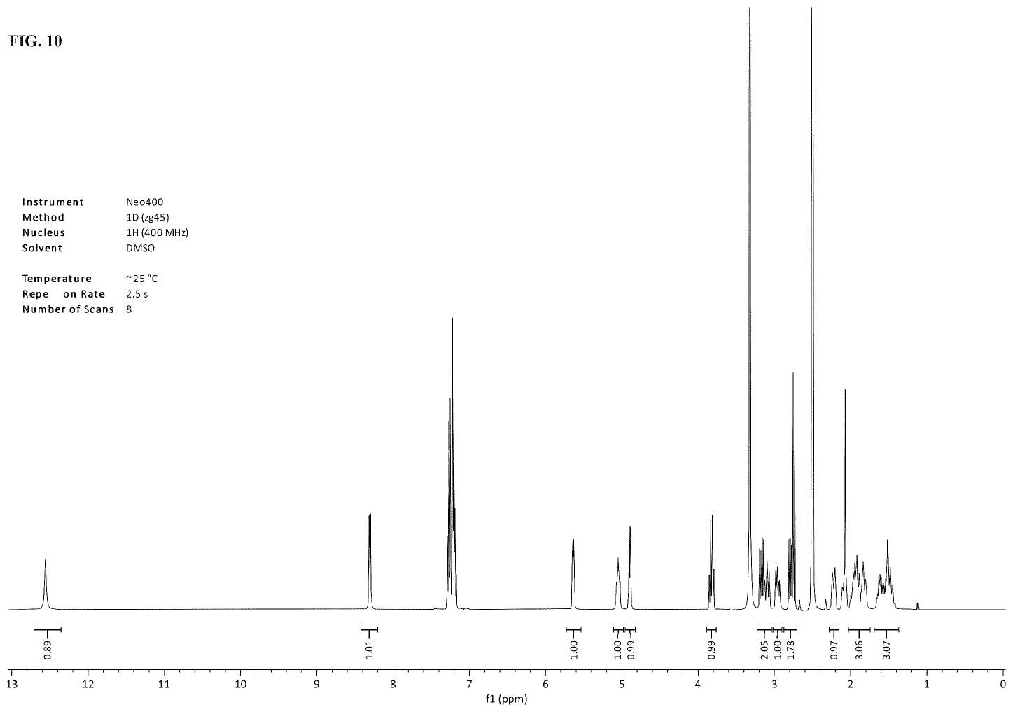

H¹NMR for (4S,7S,10aS)-4-((S)-2-mercapto-3-phenylpropanamido)-5-oxooctahydro-7H-pyrido[2,1-b][1,3]thiazepine-7-carboxylic acid is shown in FIG. 10.

MS: m/z=430.98 [M+Na]+.

Liquid chromatography of Compound 1 produced UV signals at the following retention times in Table 6 following the method outlined in the “non-chiral method for the determination of the purity of Compound 1” describe above.

TABLE 6

| | Retention | Area |
|---|---|---|
| Peak | time | (%) |
| 1 | 0.72 | 0.015 |
| 2 | 1.51 | 0.047 |
| 3 | 1.53 | 0.040 |
| 4 | 1.60 | 0.016 |
| 5 (Omapatrilat) | 1.72 | 98.929 |
| 6 | 1.90 | 0.216 |
| 7 | 1.93 | 0.569 |
| 8 | 1.96 | 0.080 |
| 9 | 1.97 | 0.037 |
| 10 | 1.99 | 0.038 |
| 11 | 2.03 | 0.012 |

Importantly, Compound 1 synthesized by the methods described herein has a significantly improved purity compared to Compound 1 provided by commercial sources. Liquid chromatography of commercially sourced Compound 1 produced UV signals at the following retention times in Table 7 following the method outlined in the “non-chiral method for the determination of the purity of Compound 1” describe above.

TABLE 7

| | Retention | Area |
|---|---|---|
| Peak | time | (%) |
| 1 (Omapatrilat) | 1.72 | 94.528 |
| 2 | 1.93 | 3.680 |
| 3 | 1.96 | 1.792 |

Table 7 shows that commercially sourced Compound 1 has a purity of less than 95% and contains each impurity in an amount greater than 1.7%, compared with the greater than 98% purity of Compound 1 and each impurity present in an amount less than 0.06%.

It was discovered upon reaction scale up that a number of impurities, including epimerization, of the final product is the largest impurity formed during the formation of Compound 1. It was further discovered that amount of the Compound 1 epimer can be controlled via careful selection of reaction time, temperature, and work up conditions.

In Table 8 the screening conditions are reported. W/M=Water/Methanol. A-B=Acid/Base The reported conversion percentages refer to the conversion to the final product Omapatrilat. % iso-OMA refers to percent of the Omapatrilat epimer in the final product. % Yield=isolated Omapatrilat. If two percentages are reported, the second refers to epimer percent (column 9) or yield (column 12) after trituration with hot-acetonitrile. The isolation of the product was performed in 4 different ways:
1) according to literature, by precipitation of the product from the reaction mixture via acidification (indicated in the table as ppt);
2) reverse precipitation, by pouring the reaction mixture into aq. HCl (Rev. ppt);
3) by extraction: dilution of the reaction mixture with DCM and acidification with 6 M HCl in IPA (extr);
4) by extraction (2): quenching with 1 M aq HCl and extracting with MeTHF (extr2).

Scheme 1: Deprotection of methyl (4S,7S,10aS)-4-((S)-2-(acetylthio)-3-phenylpropanamido)-5-oxooctahydro-7H-pyrido[2,1-b][1,3]thiazepine-7-carboxylate to give Compound 1 and the corresponding observed side products.

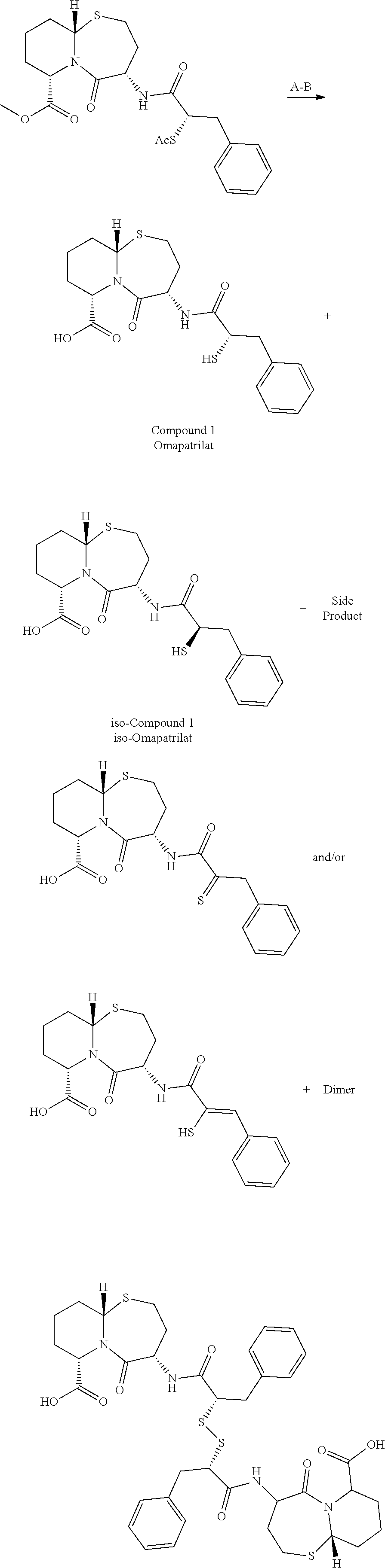

TABLE 8
Conditions screening for the deprotection of methyl (4S,7S,10aS)-4-((S)-2-(acetylthio)-
3-phenylpropanamido)-5-oxooctahydro-7H-pyrido[2,1-b][1,3]thiazepine-7-carboxylate.

| | | | | | | | | | Side | | |
|---|---|---|---|---|---|---|---|---|---|---|---|
| | Scale | | Eq | | T° C./ | Conv | isolation | % iso- | Product | Dimer | Yield |
| | (g) | A-B | A-B | Solv | time | % | mode | OMA* | % | % | % |
| 1 | 0.03 | NaOH | 7.7 | W/M | 5/2 h | 100 | ppt | — | — | — | 45 |
| 2 | 0.30 | NaOH | 7 | W/M | 5/16 h | 100 | ppt | — | — | — | 64 |
| 3 | 3.5 | NaOH | 7 | W/M | 5/16 h | 100 | ppt | 35 | | | 71 |
| 4 | 6.4 | NaOH | 3.5 | W/M | 5/16 h | 100 | ppt | 30 | 11 | — | 39 |
| 5 | 0.10 | NaOH | 5 | W/M | rt/3 h | 100 | extr. | 17 | — | — | 40 |
| 6 | 0.10 | NaOH | 5 | W/THF | rt/1 h | 100 | extr. | 14 | — | — | quant |
| 7 | 0.10 | LiOH | 2 | W/M | rt/16 h | 43 | — | — | 4 | 4 | — |
| 8 | 0.10 | LiOH | 5 | W/M | rt/16 h | 100 | extr. | 33 | 9 | — | — |
| 9 | 0.10 | LiOH | 5 | W/M | rt/3 h | 100 | extr. | 17 | — | — | 40 |
| 10 | 0.10 | LiOH | 5 | W/THF | rt/1 h | 100 | extr. | 12 | | | quant |
| 11 | 0.10 | LiOH | 5 | W/THF | rt/1 h | 100 | Rev. | 9 | — | — | 60 |
| | | | | | | | ppt | | | | |
| 12 | 0.10 | LiOH | 5 | W/THF | 60/1 h | 100 | extr. | 15 | 2 | — | 66 |
| 13 | 0.10 | LiOH | 3 | W/THF | 60/1 h | 100 | extr. | 17 | 5 | — | 71 |
| 14 | 0.50 | LiOH | 5 | W/THF | 4-17/3 h | 100 | extr. | 15/1 | 4 | — | 87/52 |
| 15 | 0.50 | LiOH | 5 | W/THF | rt/1 h | 100 | extr.** | 11/1 | — | 3 | 88/52 |
| 16 | 0.50 | LiOH | 5 | W/Me-THF | 20-60/3 h | 100 | Extr2 | 15/1 | 4 | 3 | 86/52 |
| 17 | 1.6 | LiOH | 5 | W/THF | rt/1.5 h | 100 | Extr2 | 10/0.33^{#} | — | — | 90/73 |
| 18 | 5.0 | LiOH | 5 | W/THF | rt/1.5 h | 100 | Extr2 | 12/0.33^{#} | 1 | 1 | 93/75 |
| 19 | 24.0 | LiOH | 5 | W/THF | rt/1.5 h | 100 | Extr2 | 14/0.25 | — | — | 95/75 |
| 20 | 0.10 | KOH | 5 | W/M | rt/3 h | 100 | extr. | 24 | — | — | — |
| 21 | 0.10 | K₂CO₃ | 2 | W/M | rt/16 h | 20 | extr. | — | — | 30 | — |
| 22 | 0.10 | HCl | 2 | W/M | rt/16 h | — | — | — | — | — | — |
| 23 | 0.10 | HCl | 5 | W/M | rt/16 h | — | — | — | — | — | — |
| 24 | 0.10 | LiBr | 10 | MeOH | rt/16 h | — | — | — | — | 40 | — |
| 25 | 0.10 | LiBr | 10 | ACN | rt/16 h | — | — | — | — | 20 | — |
| 26 | 0.10 | LiBr | 10 | THF | rt/16 h | — | — | — | — | 20 | — |
| 27 | 0.10 | Dowex | — | W/M | rt/16 h | — | — | — | — | — | — |

*Determined by integration of NMR signals.
**in this case extraction was performed with MeTHF instead of with DCM.
^{#}when hot trituration was performed overnight the epimer of Omapatrilat was not detectable.

Entries 1-2 show the results obtained on small scale when the epimerization of Omapatrilat was not observed. Entries 3-4 show the results of the larger batches and the presence of iso-Omapatrilat.

Hydrolysis in presence of NaOH was tested both in MeGH and in THF. It was discovered that hydrolysis in THF was three times faster than in MeGH. When entries 4-6 are compared, it is evident that shorter reaction time considerably decreases the amount of iso-Omapatrilat formed (from 30% to 14%). The same trend can be observed when entries 8-10 are compared. In this case the hydrolysis was performed in presence of LiGH and again the formation of iso-Omapatrilat decreased by about 50% (from 33% to 12%) when reducing reaction time from 16 h to 1 h.

In entry 12 and 13 the reactions were heated at 60° C. with 5 or 3 eq of LiGH. These experiments gave comparable results between each other but showed increased levels of side product formation compared to reactions run at room temperature (entry 10). However, the differences are small and the hydrolysis performed well at 60° C., indicating that an accidental increase of temperature during the reaction will not have catastrophic consequences.

In entry 14, the reaction was scaled up to 500 mg and cooled at the beginning resulting in slower conversion but comparable purity to entries 9-13.

In entry 15, the reaction was scaled up to 500 mg and performed at rt letting the temperature rise (to 25° C.) during the addition ofLiGH. In this case, the reaction was performed with Me-THF instead of DCM with similar product yields. In entry 16, the reaction was performed in Me-THF, resulting in slower conversion and heating to 60° C. was needed, resulting in slightly higher formation of side product, but phase separation was excellent.

Entries 14-16 all gave comparable results with yields around 87% and about 15% iso-Omapatrilat formation. The batches were joined and purified by hot trituration in MeCN (15 vol), resulting in 60% recovery of Omapatrilat with 99% chiral purity.

The reaction was further scaled up to 1.6 g and 5 g (entry 17-18). The reactions were perfectly reproducible and gave 90% crude recovery and about 10% iso-Omapatrilat formation. Hot trituration in MeCN (15 vol, refluxing 1h, cooling 4h), gave the desired product in 81% recovery and 75% overall yield with only 0.33% of undesired isomer. Hot trituration was also tested by refluxing overnight and cooling for 4h. In this case, iso-Omapatrilat could not be detected and product recovery was only slightly lower (75%).

The hydrolysis was also tested in presence of KOH (Entry 20) but, the reaction rate was slower, resulting in more isomerization. Hydrolysis with K₂CO₃ (entry 21) was less successful, yielding only 20% conversion to Omapatrilat and significant dimer formation. Hydrolysis with HCl (entries 22-23) was unsuccessful because the starting material precipitated in the reaction mixture. Hydrolysis with LiBr (entries 24-26, according to Tetrahedron Letters 48 (2007) 2497-2499) gave deacetylation followed by dimerization and no hydrolysis of the methyl ester was observed. Hydrolysis with acidic resin Dowex-50 was also unsuccessful.

### Example 9: Purification of (4S,7S,10aS)-4-((S)-2-mercapto-3-phenylpropanamido)-5-oxooctahydro-7H-pyrido[2,1-b][1,3]thiazepine-7-carboxylic acid (Compound 1) by hot trituration

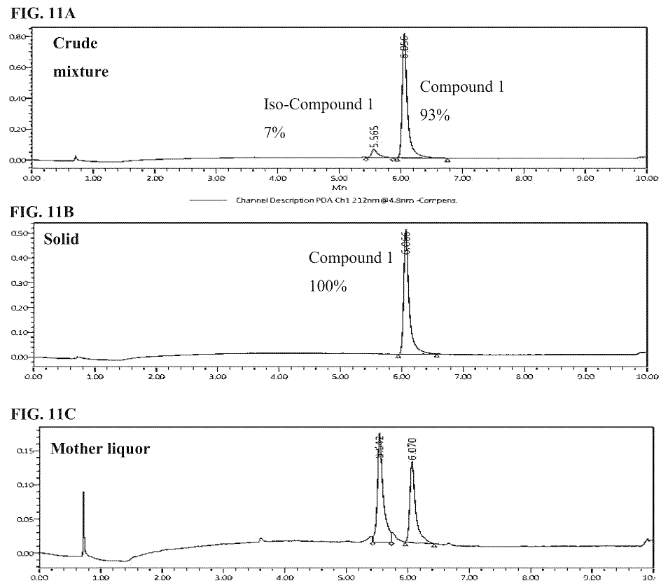

Purification of a 93:7 mixture of Omapatrilat (Compound 1): iso-Omapatrilat (iso-Compound 1) (ratio determined by integration of chiral LC-MS signals, corresponding to about 85:15 when determined by integration of ¹H-NMR signals) was tested by hot trituration from 15 vol of acetonitrile, results are reported in Table 9. Long heating time and short cooling time (entry 4) lead to the complete removal of the undesired isomer. The chiral HPLC traces of the crude mixture (FIG. 11A), precipitate (FIG. 11B), and mother liquor (FIG. 11C) corresponding to entry 4 are shown at 212 nm. This procedure was performed on 17.3 g of a 93:7 mixture of Omapatrilat (Compound 1): iso-Omapatrilat (iso-Compound 1) affording Compound 1 in 75% yield and 99.75% chiral purity.

TABLE 9
Purification of Omapatrilat by hot trituration from acetonitrile.

| | | | | | | | Omapatrilat:Iso- | Omapatrilat:Iso- | |
|---|---|---|---|---|---|---|---|---|---|
| | Sample | | Reflux | | Cooling | | Omapatrilat | Omapatrilat | |
| | size | | time | | time | | (Mother Liquor) | (solid) | Yield |
| 1 | 0.44 | g | 15 | min | 16 | h | 70:30 | 98.7:1.3 | 70% |
| 2 | 1.15 | g * | 40 | min | 16 | h | 49:51 | 99.2:0.8 | 60% |
| 3 | 4.5 | g | 60 | min | 4 | h | 35:65 | 99.7:0.3 | 81% |
| 4 | 0.50 | g | 16 | h | 4 | h | 43:57 | 100:0 | 75% |
| 5 | 17.3 | g | 2 | h | 4 | h | 40:60 | 99.7:0.3 | 75% |

* the starting material had 11% iso-Omapatrilat.

### Example 10: Determination of Side Product Identity

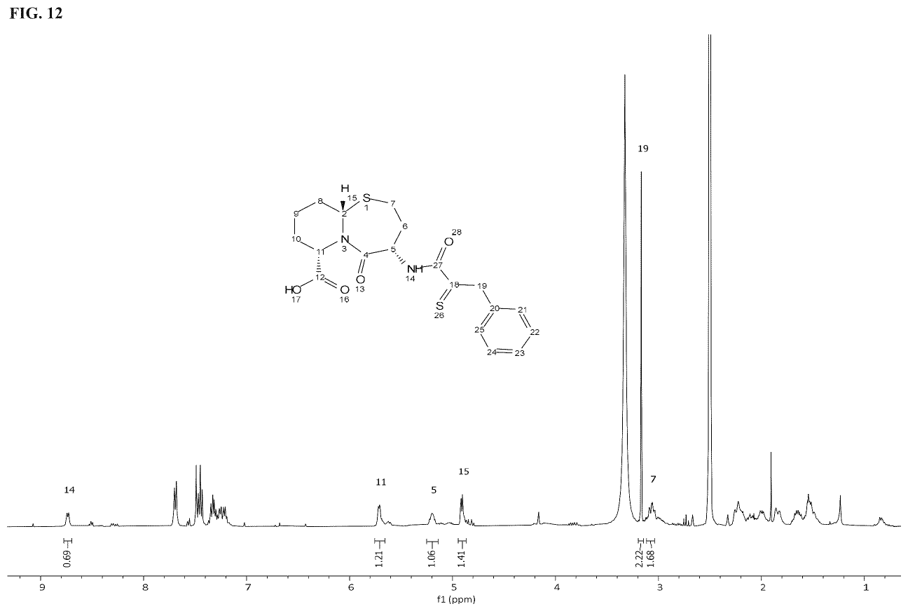

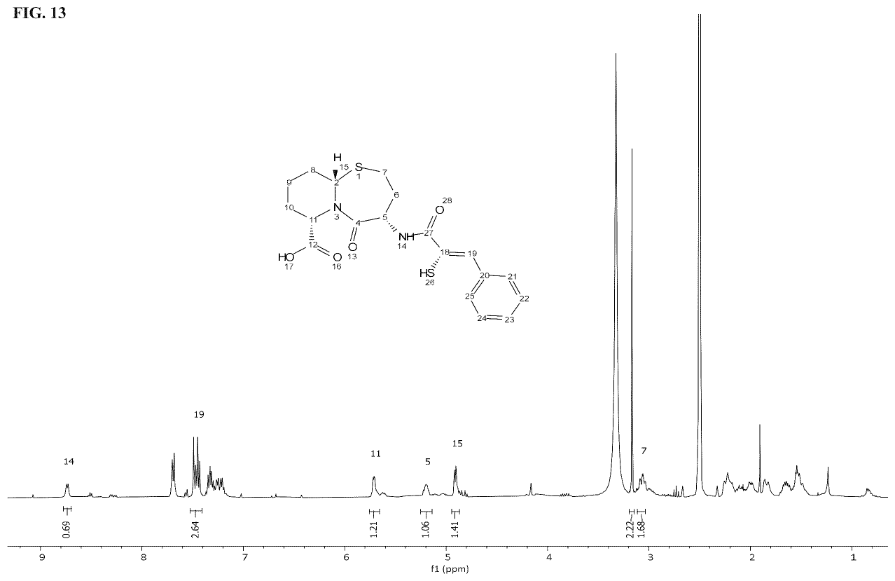

The side product shown in Table 8, entries 4, 7, 8, 12-14, 16, and 18 was isolated and determined to have a MS: m/z=429.2 [M+Na]+, which compared to the mass of the desired product Compound 1 is M-2. The mass spec data, along with H¹NMR, suggest the side product is a thioketone (FIG. 12) or an unsaturated thiol (FIG. 13).

The examples and embodiments described herein are for illustrative purposes only and various modifications or changes suggested to persons skilled in the art are to be included within the spirit and purview of this application and scope of the appended claims.

1-114. (canceled)

115. A purified compound having the structure of (4S,7S,10aS)-4-((S)-2-mercapto-3-phenylpropanamido)-5-oxooctahydro-7H-pyrido[2,1-b][1,3]thiazepine-7-carboxylic acid (Compound 1) or a pharmaceutically acceptable salt thereof. 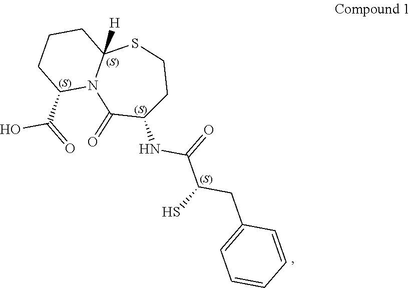
wherein
(i) the compound purity is greater than 97.0% as determined by chromatographic analysis at 215 nm;
(ii) the total amount of any one impurity is less than 1.5% as determined by chromatographic analysis at 215 nm;
(iii) the total content of all impurities is less than 3.0% as determined by chromatographic analysis at 215 nm; or
(iv) combinations thereof, wherein 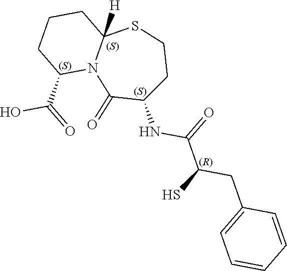
is present in an amount less than 1% (w/w).

116. The purified compound of claim 115, wherein the compound has an optical purity of greater than about 98% enantiomeric excess.

117. The purified compound of claim 115, wherein the impurity or impurities comprises one or more of the impurities selected from the group consisting of

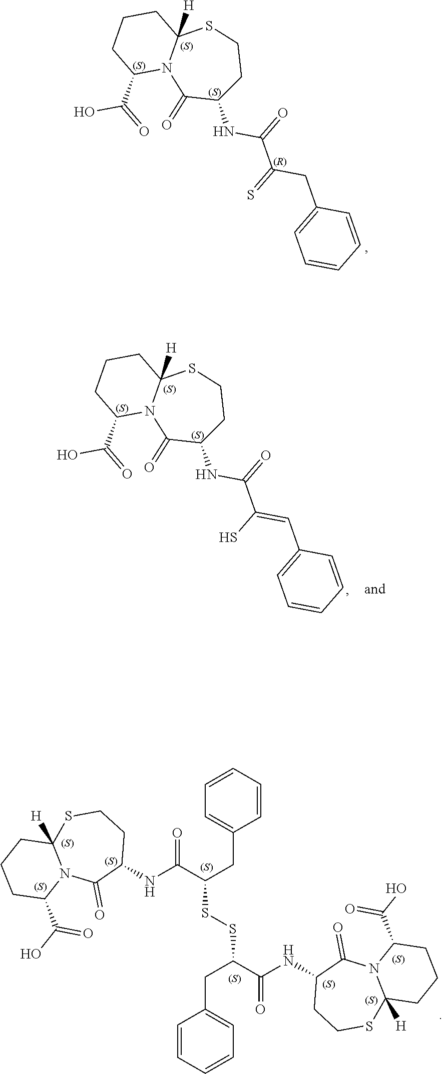

118. A pharmaceutical composition comprising the purified compound of claim 115, or a pharmaceutically acceptable salt thereof, and at least one pharmaceutically acceptable excipient.

119. A process for the preparation of (4S,7S,10aS)-4-((S)-2-mercapto-3-phenylpropanamido)-5-oxooctahydro-7H-pyrido[2,1-b][1,3]thiazepine-7-carboxylic acid (Compound 1) or a salt thereof: 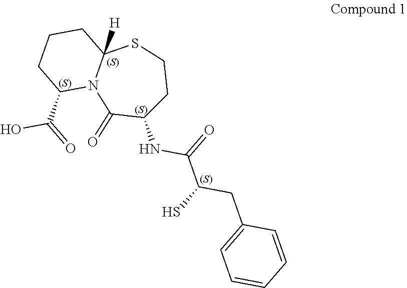
comprising the steps of:
(i)(a) reacting Compound 4 or a salt thereof: 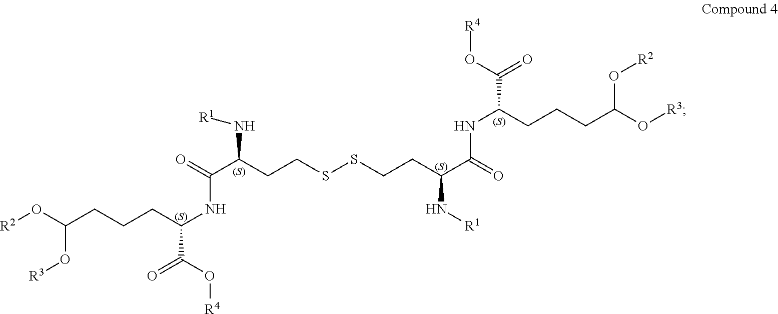
with a reagent that cleaves the disulfide bond to produce a thiol monomer, wherein
R¹ is a benzyl carbamate; and
R² and R³ are taken together to form dioxolane;
R⁴ is C₁₋₃ alkyl;
(i)(b) subjecting the monomer from step (i)(a) to an acid catalyzed cyclization reaction in a suitable solvent to provide Compound 5 or a salt thereof: 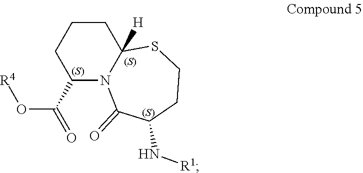
(ii) reacting Compound 5 with a suitable reagent to provide Compound 6 or a salt thereof: 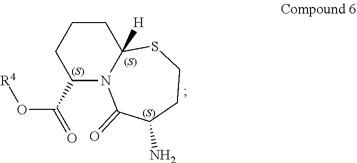
(iii) reacting Compound 6 with Compound 7 or a salt thereof: 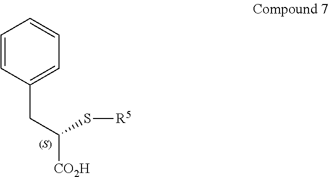
in the presence of a coupling reagent, wherein
R⁵ is —C(O)-methyl;
to provide Compound 8 or a salt thereof: 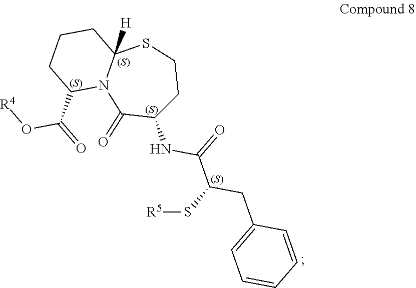
(iv) treating Compound 8 to provide (4S,7S,10aS)-4-((S)-2-mercapto-3-phenylpropanamido)-5-oxooctahydro-7H-pyrido[2,1-b][1,3]thiazepine-7-carboxylic acid (Compound 1) or a salt thereof.

120. The process of claim 119, wherein Compound 4 is Compound 4a:

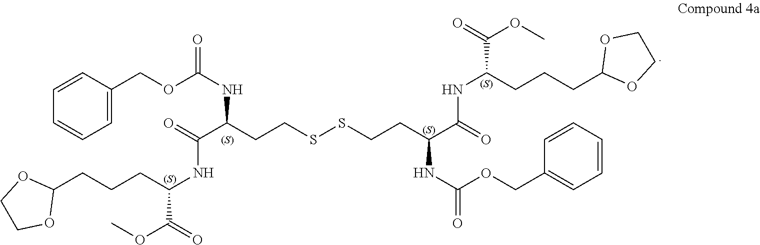

121. The process of claim 119, wherein the suitable solvent of step (i)(b) comprises water.

122. The process of claim 120, wherein the water comprises less than about 10% of the solvent by volume.

123. The process of claim 119, wherein the acid catalyst of step (i)(b) comprises trifluoroacetic acid, chlorosulfonic acid, p-toluenesulfonic acid, methanesulfonic acid, trifluoromethanesulfonic acid, trimethylsilyl trifluoromethanesulfonate, trimethylsilyl methanesulfonate or Amberlyst.

124. The process of claim 119, wherein the acid catalyst of step (i)(b) is trifluoroacetic acid.

125. The process of claim 119, wherein the coupling reagent of step (iii) is propylphosphonic anhydride (T3P) or benzotriazol-1-yloxytripyrrolidinophosphonium hexafluorophosphate (PyBOP).

126. The process of claim 119, wherein step (iii) further comprises purifying Compound 8 via crystallization or precipitation.

127. The process of claim 119, wherein (4S,7S,10aS)-4-((S)-2-mercapto-3-phenylpropanamido)-5-oxooctahydro-7H-pyrido[2,1-b][1,3]thiazepine-7-carboxylic acid (Compound 1) is purified via a trituration.

128. The process of claim 127, wherein the trituration comprises acetonitrile.

129. The process of claim 127, wherein the trituration comprises refluxing acetonitrile.

130. The process of claim 119, wherein Compound 4: 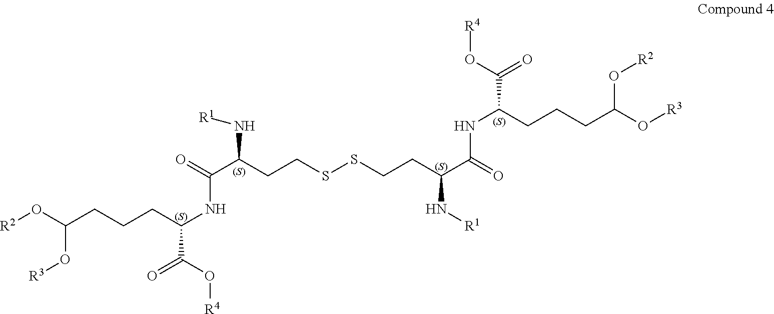
is prepared by reacting Compound 2: 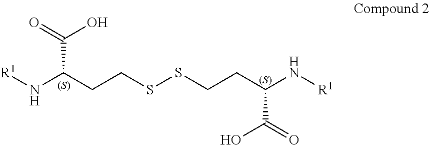
with Compound 3: 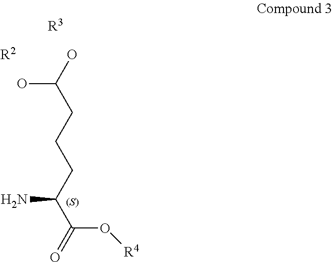
in the presence of a coupling reagent and in a suitable solvent.

131. The process of claim 119, further comprising purifying Compound 4 by crystallization.

132. The process of claim 119, wherein the optical purity of Compound 7 is greater than about 90% enantiomeric excess.

133. The process of claim 119, wherein the (4S,7S,10aS)-4-((S)-2-mercapto-3-phenylpropanamido)-5-oxooctahydro-7H-pyrido[2,1-b][1,3]thiazepine-7-carboxylic acid (Compound 1) is prepared with an optical purity of is greater than about 97% enantiomeric excess.

134. A compound with the structure: 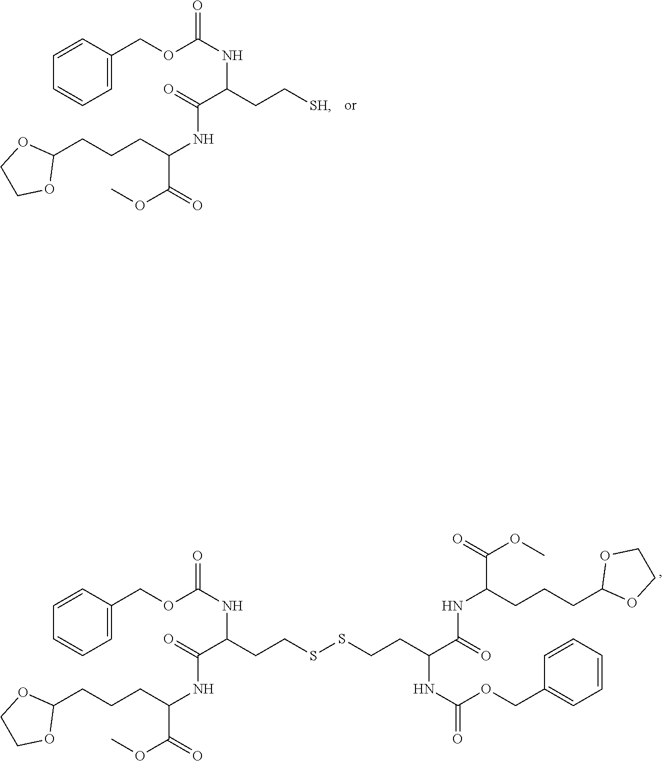 [SMILES: COC(=O)C(CCCC1OCCO1)NC(=O)C(CCS)NC(=O)OCc1ccccc1 ; COC(=O)C(CCCC1OCCO1)NC(=O)C(CCSSCCC(NC(=O)OCc1ccccc1)C(=O)NC(CCCC1OCCO1)C(=O)OC)NC(=O)OCc1ccccc1]
or a salt thereof.
